(19) United States
(12) Patent Application Publication
(10) Pub. No.: US 2011/0118238 A1
(43) Pub. Date: May 19, 2011

(54) 2-ANILINOPURIN-8-ONES AS INHIBITORS OF TTK/MPS1 FOR THE TREATMENT OF PROLIFERATIVE DISORDERS

(75) Inventors: David Michael Andrews, Cheshire (GB); Clifford David Jones, Cheshire (GB); Iain Simpson, Cheshire (GB); Richard Andrew Ward, Cheshire (GB)

(73) Assignee: ASTRAZENECA AB, Södertälje (SE)

(21) Appl. No.: 12/674,749

(22) PCT Filed: Aug. 20, 2008
(86) PCT No.: PCT/GB08/50724

§ 371 Date: Jul. 13, 2010

Related U.S. Application Data
(60) Provisional application No. 60/957,508, filed on Aug. 23, 2007.

(51) Int. Cl. A61K 31/5513; C07D 473/00; A61K 31/522; A61K 31/5377; A61K 31/541; A61P 35/00

(52) U.S. Cl. 514/221; 544/276; 544/118; 544/58.2; 540/569; 514/263.22; 514/263.3; 514/252.16; 514/234.2; 514/263.2; 514/228.5

(57) ABSTRACT

This invention relates to chemical compounds of the formula (I), or a pharmaceutically acceptable salt thereof, which possess inhibitory activity against the spindle checkpoint kinase: Tyrosine Threonine Kinase (TTK)/monopolar spindle 1 (Mps1) and are accordingly useful for their anti-cancer effect in a warm-blooded animal such as man. The invention also relates to processes for the manufacture of said chemical compounds, to pharmaceutical compositions containing them, and to their use in the manufacture of a medicament for the treatment of conditions mediated by TTK/Mps1, for use either alone or in combination with other anti-pro liferative agents.

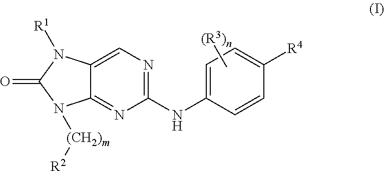

The present invention relates to chemical compounds, or a pharmaceutically acceptable salt thereof, which possess inhibitory activity against the spindle checkpoint kinase: Tyrosine Threonine Kinase (TTK)/monopolar spindle 1 (Mps1). TTK is the human homologue of the S. cerevisiae kinase Mps1. The chemical compounds of the present invention and the pharmaceutically acceptable salts thereof are accordingly useful for their anti-cancer effect in a warm-blooded animal such as man. The invention also relates to processes for the manufacture of said chemical compounds, to pharmaceutical compositions containing them, and to their use in the manufacture of a medicament for the treatment of conditions mediated by TTK/Mps1, for use either alone or in combination with other anti-proliferative agents.

Among the therapeutic agents used to treat cancer are the taxanes and vinca alkaloids which act on microtubules either stabilising or destabilising microtubule dynamics. These perturb normal mitotic spindle function, preventing correct chromosome attachment and inducing mitotic arrest. This arrest is enforced by the spindle assembly checkpoint and prevents separation of sister chromatids to form the two daughter cells. Prolonged arrest in mitosis forces a cell into mitotic exit without cytokinesis or into mitotic catastrophe leading to cell death.

Although mitotic agents are broadly used in the treatment of solid tumours the side effects associated with these agents and the resistance of many types of tumours to the current therapies calls for the development of new pharmaceutical compositions in the treatment of cancer.

The roles that the genes involved in the spindle assembly checkpoint play in normal development and their potential roles in disease such as cancer have been widely studied (Weaver B A and Cleveland D W, Cancer cell, 2005, 8, 7-12; Musacchio A and Salmon E D Nat. Rev. Mol. Cell. Biol., 2007, 8, 379-393). Many of the components are phosphorylated during mitosis and several of them are kinases, one of which is the dual specificity kinase TTK. TTK expression is associated with highly proliferating cells and tissues with overexpression observed in a number of cancer cell lines and tumour types and silencing of TTK in several species leads to failure of cells to arrest in mitosis in response to spindle poisons indicating its essential function in spindle assembly checkpoint signalling (Abrieu A et al, Cell, 2001, 106, 83-93; Stucke, V M et al EMBO J., 2002, 21, 1723-1732).

These findings suggest that pharmacological inhibitors of TTK and other components of the spindle assembly checkpoint should be of therapeutic value for treatment of proliferative disease including solid tumours such as carcinomas and sarcomas and the leukaemias and lymphoid malignancies. In addition TTK inhibitors should be useful in the treatment of other disorders associated with uncontrolled cellular proliferation.

Therefore in the first aspect of the invention there is provided a compound of formula (I):

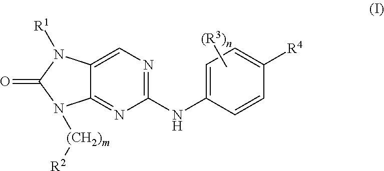

wherein:

R¹ is selected from C₁₋₄alkyl, cyclopropyl, cyclopropylmethyl and cyclobutyl; wherein said cyclopropyl may be optionally substituted by methyl; and wherein R¹ may be optionally substituted by one or more R⁵;

m is 0 or 1;

R² is selected from C₁₋₆alkyl, C₂₋₆alkenyl, C₂₋₆alknyl, C₃₋₆cycloalkyl, cyclopentenyl, cyclohexenyl, oxetanyl, tetrahydropyranyl, tetrahydrofuranyl, oxepanyl, azetidinyl, pyrrolidinyl, piperidinyl and azepanyl; wherein R² may be optionally substituted on carbon by one or more R⁶; and wherein if R² contains a ring —NH— moiety, that nitrogen may be optionally substituted by R⁷;

R³ is independently selected from fluoro, chloro, bromo, cyano, methoxy, ethoxy, trifluoromethoxy, methyl, ethyl, trifluoromethyl, ethenyl, ethynyl, cyclopropyl, methylthio, ethylthio, N-methylamino, N,N-dimethylamino, amino and methylsulfonyloxy;

n is an integer selected from 0 to 3; wherein the values of R³ may be the same or different;

R⁴ is -L-R⁸ or R⁹;

L is selected from ethynylene, ethenylene, cyclopropyl and —X—C₁₋₂alkylene-; wherein X is a direct bond, —O—, —S—, —NH—, —OS(O)₂—, —N(CH₃)— or —N(CH₂R¹⁰)—; and wherein L may be optionally substituted on carbon by one or more fluoro;

R⁵ is cyano or fluoro;

R⁶ is selected from C₁₋₃alkyl, C₁₋₃alkoxy, N—(C₁₋₃alkyl)amino, N,N—(C₁₋₃alkyl)₂-amino, hydroxy, amino, fluoro and cyano;

R⁷ is selected from C₁₋₃alkyl, cyclopropyl, C₁₋₃alkanoyl and C₁₋₃alkylsulfonyl;

R⁸ and R¹⁰ are each independently selected from chloro, bromo, iodo, cyano, nitro, mercapto, sulfo, hydroxy, carboxy, amino, carbamoyl, sulfamoyl, C₂₋₆alkyl, C₂₋₆alkenyl, C₂₋₆alkynyl, C₁₋₆alkoxy, C₁₋₆alkylsulfonyloxy, N—(C₁₋₆alkyl)sulfamoyloxy, N,N—(C₁₋₆alkyl)₂sulfamoyloxy, C₁₋₆alkoxycarbonyl, C₁₋₆alkanoyl, C₁₋₆alkanoyloxy, N—(C₁₋₆alkyl)amino, N,N—(C₁₋₆alkyl)₂-amino, N—(C₁₋₆alkanoyl)-N—(R¹¹)amino, N—(C₁₋₆alkoxycarbonyl)-N—(R¹²)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—[(C₁₋₆alkyl)sulfonyl]-N—(R¹³)amino, (N,N—(R¹⁴)(R¹⁵)sulfamoyl)-N—(R¹⁶)amino, 3,3-(R¹⁷)(R¹⁸)-1-(R¹⁹)ureido, carbocyclyl-R²⁰—, heterocyclyl-R²¹— and (C₁₋₆alkyl)-S(O)ₐ— wherein a is 0 to 2, wherein R⁸ and R¹⁰ may be optionally substituted on carbon by one or more R²²; and wherein if said heterocyclyl has an —NH— moiety, that nitrogen may be optionally substituted by R²³;

R⁹ is selected from carboxy, carbamoyl, sulfamoyl, C₃₋₆alkyl, C₃₋₆alkenyl, C₃₋₆alkynyl, C₃₋₆alkoxy, C₁₋₆alkylsulfonyl, C₁₋₆alkylsulfinyl, C₃₋₆alkylsulfanyl, C₂₋₆alkylsulfonyloxy, N—(C₁₋₆alkyl)sulfamoyloxy, N,N—(C₁₋₆alkyl)₂sulfamoyloxy, C₁₋₆alkoxycarbonyl, C₁₋₆alkanoyl, C₁₋₆alkanoyloxy, N—(C₂₋₆alkyl)amino, N,N—(C₂₋₆alkyl)₂amino, N—(C₁₋₆alkanoyl)-N—(R²⁴)amino, N—(C₁₋₆alkoxycarbonyl)-N—(R²⁵)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—[(C₁₋₆alkyl)sulfonyl]-N—(R²⁶)amino, (N,N—(R²⁷)(R²⁸)sulfamoyl)-N—(R²⁹)amino, 3,3-(R³⁰)(R³¹)-1-(R³²)ureido, C₄₋₁₂carbocyclyl-R³³— and heterocyclyl-R³⁴—; wherein R⁹ may be optionally substituted on carbon by one or more R³⁵, and wherein if said heterocyclyl has an —NH— moiety, that nitrogen may be optionally substituted by R³⁶;

R²² and R³⁵ are independently selected from halo, cyano, nitro, mercapto, sulfo, hydroxy, carboxy, amino, carbamoyl, sulfamoyl, C₁₋₆alkyl, C₂₋₆alkenyl, C₂₋₆alkynyl, C₁₋₆alkoxy, C₁₋₆alkylsulfonyloxy, N—(C₁₋₆alkyl)sulfamoyloxy, N,N—(C₁₋₆alkyl)₂sulfamoyloxy, C₁₋₆alkoxycarbonyl, C₁₋₆alkanoyl, C₁₋₆alkanoyloxy, N—(C₁₋₆alkyl)amino, N,N—(C₁₋₆alkyl)₂amino, N—(C₁₋₆alkanoyl)-N—(R³⁷)amino, N—(C₁₋₆alkoxycarbonyl)-N—(R³⁸)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—[(C₁₋₆alkyl)sulfonyl]-N—(R³⁹)amino, (N,N—(R⁴⁰)(R⁴¹)sulfamoyl)-N—(R⁴²)amino, 3,3-(R⁴³)(R⁴⁴)-1-(R⁴⁵)ureido, carbocyclyl-R⁴⁶—, heterocyclyl-R⁴⁷— and (C₁₋₆alkyl)-S(O)ₐ—wherein a is 0 to 2; wherein R²² and R³⁵ may be optionally substituted on carbon by one or more R⁴⁸; and wherein if said heterocyclyl has an —NH— moiety, that nitrogen may be optionally substituted by R⁴⁹;

R²³ and R³⁶ are independently selected from C₁₋₆alkyl, C₂₋₆alkenyl, C₂₋₆alkynyl, C₁₋₆alkoxycarbonyl, C₁₋₆alkanoyl, carbamoyl, N—(C₁₋₆alkyl)carbamoyl, N,N—(C₁₋₆alkyl)₂carbamoyl, sulfamoyl, N—(C₁₋₆alkyl)sulfamoyl, N,N—(C₁₋₆alkyl)₂sulfamoyl, carbocyclyl-R⁵⁰—, heterocyclyl-R⁵¹—, and (C₁₋₆alkyl)-S(O)ₐ— wherein a is 1 or 2; wherein R²³ and R³⁶ may be independently optionally substituted on carbon by one or more R⁵²; and wherein if said heterocyclyl contains an —NH— moiety that nitrogen may be optionally substituted by R⁵³;

R²⁰ and R²¹ are each independently selected from a direct bond, —O—, —N(R⁵⁴)—, —C(O)—, —N(R⁵⁵)C(O)—, —C(O)N(R⁵⁶)—, —SO₂N(R⁵⁷)—, —N(R⁵⁸)—C(O)—N(R⁵⁹)—, —OS(O)₂—, —S(O)₂O—, —N(R⁶⁰)S(O)₂N(R⁶¹)—, —N(R⁶²)SO₂— and —S(O)ₐ— wherein a is 0 to 2;

R³³ and R³⁴ are each independently selected from a direct bond, —O—, —N(R⁶³)—, —C(O)—, —N(R⁶⁴)C(O)—, —C(O)N(R⁶⁵)—, —SO₂N(R⁶⁶)—, —N(R⁶⁷)—C(O)—N(R⁶⁸)—, —OS(O)₂—, —S(O)₂O—, —N(R⁶⁹)S(O)₂N(R⁷⁰)—, —N(R⁷¹)SO₂— and —S(O)ₐ— wherein a is 0 to 2;

R⁴⁶ and R⁴⁷ are each independently selected from a direct bond, —O—, —N(R⁷²)—, —C(O)—, —N(R⁷³)C(O)—, —C(O)N(R⁷⁴)—, —SO₂N(R⁷⁵)—, —N(R⁷⁶)—C(O)—N(R⁷⁷)—, —OS(O)₂—, —S(O)₂O—, —N(R⁷⁸)S(O)₂N(R⁷⁹)—, —N(R⁸⁰)SO₂— and —S(O)ₐ— wherein a is 0 to 2;

R⁵⁰ and R⁵¹ are each independently selected from a direct bond, —C(O)—, —N(R⁸¹)C(O)—, —N(R⁸²)SO₂—, —O—C(O)— and —S(O)ₐ— wherein a is 1 or 2;

R⁴⁸ and R⁵² are each independently selected from fluoro, chloro, cyano, nitro, hydroxy, trifluoromethoxy, trifluoromethyl, amino, carboxy, sulfo, carbamoyl, mercapto, sulfamoyl, methyl, ethyl, ethenyl, methoxy, ethoxy, formyl, acetyl, acetoxy, N-methylamino, N-ethylamino, N,N-dimethylamino, N,N-diethylamino, N-ethyl-N-methylamino, N-formylamino, N-acetylamino, N-methylcarbamoyl, N-ethylcarbamoyl, N,N-dimethylcarbamoyl, N,N-diethylcarbamoyl, N-ethyl-N-methylcarbamoyl, methylsulfanyl, ethylsulfanyl, methylsulfinyl, ethylsulfinyl, methylsulfonyl, methylsulfonyloxy, ethylsulfonyl, ethylsulfonyloxy, methoxycarbonyl, ethoxycarbonyl, N-methylsulfamoyl, N-ethylsulfamoyl, N,N-dimethylsulfamoyl, N,N-diethylsulfamoyl and N-ethyl-N-methylsulfamoyl;

R⁴⁹ and R⁵³ are each independently selected from C₁₋₆alkyl, C₃₋₆cycloalkyl, C₁₋₆alkanoyl, C₁₋₆alkylsulfonyl, C₁₋₆alkoxycarbonyl, carbamoyl, N—(C₁₋₆alkyl)carbamoyl, N,N—(C₁₋₆alkyl)₂carbamoyl, benzyl, benzyloxycarbonyl, benzoyl and phenylsulfonyl;

R¹¹, R¹², R¹³, R¹⁴, R¹⁵, R¹⁶, R¹⁷, R¹⁸, R¹⁹, R⁵⁴, R⁵⁵, R⁵⁶, R⁵⁷, R⁵⁸, R⁵⁹, R⁶⁰, R⁶¹ and R⁶² are each independently hydrogen or a group selected from C₁₋₃alkyl and cyclopropyl wherein said group may be optionally substituted on Carbon by one or more R²²;

R²⁴, R²⁵, R²⁶, R²⁷, R²⁸, R²⁹, R³⁰, R³¹, R³², R⁶³, R⁶⁴, R⁶⁵, R⁶⁶, R⁶⁷, R⁶⁸, R⁶⁹, R⁷⁰ and R⁷¹ are each independently hydrogen or a group selected from C₁₋₃alkyl and cyclopropyl wherein said group may be optionally substituted on carbon by one or more R³⁵;

R³⁷, R³⁸, R³⁹, R⁴⁰, R⁴¹, R⁴², R⁴³, R⁴⁴, R⁴⁵, R⁷², R⁷³, R⁷⁴, R⁷⁵, R⁷⁶, R⁷⁷, R⁷⁸, R⁷⁹ and R⁸⁰ are each independently hydrogen or a group selected from C₁₋₃alkyl and cyclopropyl wherein said group may be optionally substituted on carbon by one or more R⁴⁸;

R⁸¹ and R⁸² are each independently hydrogen or a group selected from C₁₋₃alkyl and cyclopropyl wherein said group may be optionally substituted on carbon by one or more R⁵²;

or a pharmaceutically acceptable salt thereof;
wherein the compound of formula (I) is other than:
2-{[4-(4-acetylpiperazin-1-yl)phenyl]amino}-7-methyl-9-pentan-3-yl-7,9-dihydro-8H-purin-8-one or
7-methyl-2-{[4-(4-methylpiperazin-1-yl)phenyl]amino}-9-pentan-3-yl-7,9-dihydro-8H-purin-8-one;
   or a pharmaceutically acceptable salt thereof.

Therefore, in one aspect of the invention there is provided a compound of formula (I):

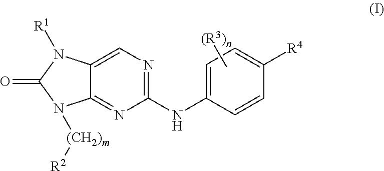

wherein:

R¹ is selected from C₁₋₄alkyl, cyclopropyl, cyclopropylmethyl and cyclobutyl; wherein said cyclopropyl may be optionally substituted by methyl; and wherein R¹ may be optionally substituted by one or more R⁵;

m is 0 or 1;

R² is C₁₋₆alkyl, C₂₋₆alkenyl, C₂₋₆alknyl, C₃₋₆cycloalkyl, cyclopentenyl, cyclohexenyl, oxetanyl, tetrahydropyranyl, tetrahydropyranyl, oxepanyl, azetidinyl, pyrrolidinyl, piperidinyl and azepanyl; wherein R² may be optionally substituted on carbon by one or more R⁶; and wherein if R² contains a ring —NH— moiety, that nitrogen may be optionally substituted by R⁷;

R³ is independently selected from fluoro, chloro, bromo, cyano, methoxy, ethoxy, trifluoromethoxy, methyl, ethyl, trifluoromethyl, ethenyl, ethynyl, cyclopropyl, methylthio, ethylthio, N-methylamino, N,N-dimethylamino, amino and methylsulfonyloxy;

n is an integer selected from 0 to 3; wherein the values of R³ may be the same or different;

R⁴ is -L-R⁸ or R⁹;

L is selected from ethynylene, ethenylene, cyclopropyl and —X—C₁₋₂alkylene-; wherein X is a direct bond, —O—, —S—, —NH—, —OS(O)₂—, —N(CH₃)— or —N(CH₂R¹⁰)—; and wherein L may be optionally substituted on carbon by one or more fluoro;

R⁵ is cyano or fluoro;

R⁶ is selected from C₁₋₃alkyl, C₁₋₃alkoxy, N—(C₁₋₃alkyl)amino, N,N—(C₁₋₃alkyl)₂amino, hydroxy, amino, fluoro and cyano;

R⁷ is selected from C₁₋₃alkyl, cyclopropyl, C₁₋₃alkanoyl and C₁₋₃alkylsulfonyl;

R⁸ and R¹⁰ are each independently selected from chloro, bromo, iodo, cyano, nitro, mercapto, sulfo, hydroxy, carboxy, amino, carbamoyl, sulfamoyl, C₂₋₆alkyl, C₂₋₆alkenyl, C₂₋₆ alkynyl, C₁₋₆alkoxy, C₁₋₆alkylsulfonyloxy, N—(C₁₋₆alkyl)sulfamoyloxy, N,N—(C₁₋₆alkyl)₂sulfamoyloxy, C₁₋₆alkoxycarbonyl, C₁₋₆alkanoyl, C₁₋₆alkanoyloxy, N—(C₁₋₆alkyl)amino, N,N—(C₁₋₆alkyl)₂amino, N—(C₁₋₆alkanoyl)-N—(R¹¹)amino, N—(C₁₋₆alkoxycarbonyl)-N—(R¹²)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—[(C₁₋₆alkyl)sulfonyl]-N—(R¹³)amino, (N,N—(R¹⁴)(R¹⁵)sulfamoyl)-N—(R¹⁶)amino, 3,3-(R¹⁷)(R¹⁸)-1-(R¹⁹)ureido, carbocyclyl-R²⁰—, heterocyclyl-R²¹, and (C₁₋₆alkyl)-S(O)ₐ—wherein a is 0 to 2, wherein R⁸ and R¹⁰ may be optionally substituted on carbon by one or more R²²; and wherein if said heterocyclyl has an —NH— moiety, that nitrogen may be optionally substituted by R²³;

R⁹ is selected from carboxy, carbamoyl, sulfamoyl, C₃₋₆alkyl, C₃₋₆alkenyl, C₃₋₆alkynyl, C₃₋₆alkoxy, C₁₋₆alkylsulfonyl, C₁₋₆alkylsulfinyl, C₃₋₆alkylsulfanyl, C₂₋₆alkylsulfonyloxy, N—(C₁₋₆alkyl)sulfamoyloxy, N—(C₁₋₆alkyl)₂sulfamoyloxy, C₁₋₆alkoxycarbonyl, C₁₋₆alkanoyl, C₁₋₆alkanoyloxy, N—(C₂₋₆alkyl)amino, N,N—(C₂₋₆alkyl)₂amino, N—(C₁₋₆alkanoyl)-N—(R²⁴)amino, N—(C₁₋₆alkoxycarbonyl)-N—(R²⁵)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—[(C₁₋₆alkyl)sulfonyl]-N—(R²⁶)amino, (N,N—(R²⁷)(R²⁸)sulfamoyl)-N—(R²⁹)amino, 3,3-(R³⁰)(R³¹)-1-(R³²)ureido, C₄₋₁₂carbocyclyl-R³³—, heterocyclyl-R³⁴—, wherein R⁹ may be optionally substituted on carbon by one or more R³⁵, and wherein if said heterocyclyl has an —NH— moiety, that nitrogen may be optionally substituted by R³⁶;

R²² and R³⁵ are independently selected from halo, cyano, nitro, mercapto, sulfo, hydroxy, carboxy, amino, carbamoyl, sulfamoyl, C₁₋₆alkyl, C₂₋₆alkenyl, C₂₋₆alkynyl, C₁₋₆alkoxy, C₁₋₆alkylsulfonyloxy, N—(C₁₋₆alkyl)sulfamoyloxy, N,N—(C₁₋₆alkyl)₂sulfamoyloxy, C₁₋₆alkoxycarbonyl, C₁₋₆alkanoyl, C₁₋₆alkanoyloxy, N—(C₁₋₆alkyl)amino, N,N—(C₁₋₆alkyl)₂amino, N—(C₁₋₆alkanoyl)-N—(R³⁷)amino, N—(C₁₋₆alkoxycarbonyl)-N—(R³⁸)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—[(C₁₋₆alkyl)sulfonyl]-N—(R⁴⁹)amino, (N,N—(R⁴⁰)(R⁴¹)sulfamoyl)-N—(R⁴²)amino, 3,3-(R⁴³)(R⁴⁴) 1 (R⁴⁵)ureido, carbocyclyl-R⁴⁶—, heterocyclyl-R⁴⁷, and (C₁₋₆alkyl)-S(O)ₐ— wherein a is 0 to 2, wherein R²² and R³⁵ may be optionally substituted on carbon by one or more R⁴⁸; and wherein if said heterocyclyl has an —NH— moiety, that nitrogen may be optionally substituted by R⁴⁹;

R²³ and R³⁶ are independently selected from C₁₋₆alkyl, C₂₋₆alkenyl, C₂₋₆alkynyl, C₁₋₆alkoxycarbonyl, C₁₋₆alkanoyl, carbamoyl, N—(C₁₋₆alkyl)carbamoyl, N,N—(C₁₋₆alkyl)₂carbamoyl, sulfamoyl, N—(C₁₋₆alkyl)sulfamoyl, N,N—(C₁₋₆alkyl)₂sulfamoyl, carbocyclyl-R⁵⁰—, heterocyclyl-R⁵¹—, and (C₁₋₆alkyl)-S(O)ₐ— wherein a is 1 or 2; wherein R²³ and R³⁶ may be independently optionally substituted on carbon by one or more R⁵²; and wherein if said heterocyclyl contains an —NH— moiety that nitrogen may be optionally substituted by R⁵³;

R²⁰ and R²¹ are each independently selected from a direct bond, —O—, —N(R⁵⁴)—, —C(O)—, —N(R⁵⁵)C(O)—, —C(O)N(R⁵⁶)—, —SO₂N(R⁵⁷)—, —N(R⁵⁸)—C(O)—N(R⁵⁹)—, —OS(O)₂—, —S(O)₂O—, —N(R⁶⁰)S(O)₂N(R⁶¹)—, —N(R⁶²)SO₂— and —S(O)ₐ— wherein a is 0 to 2;

R³³ and R³⁴ are each independently selected from a direct bond, —O—, —N(R⁶³)—, —C(O)—, —N(R⁶⁴)C(O)—, —C(O)N(R⁶⁵)—, —SO₂N(R⁶⁶)—, —N(R⁶⁷)—C(O)—N(R⁶⁸)—, —OS(O)₂—, —S(O)₂O—, —N(R⁶⁹)S(O)₂N(R⁷⁰)—, —N(R⁷¹)SO₂— and —S(O)ₐ— wherein a is 0 to 2;

R⁴⁶ and R⁴⁷ are each independently selected from a direct bond, —O—, —N(R⁷²)—, —C(O)—, —N(R⁷³)C(O)—, —C(O)N(R⁷⁴)—, —SO₂N(R⁷⁵)—, —N(R⁷⁶)—C(O)—N(R⁷⁷)—, —OS(O)₂—, —S(O)₂O—, —N(R⁷⁸)S(O)₂N(R⁷⁹)—, —N(R⁸⁰)SO₂— and —S(O)ₐ— wherein a is 0 to 2;

R⁵⁰ and R⁵¹ are each independently selected from a direct bond, —C(O)—, —N(R⁸¹)C(O)—, —N(R⁸²)SO₂—, —O—C(O)— and —S(O)ₐ— wherein a is 1 or 2;

R⁴⁸ and R⁵² are each independently selected from fluoro, chloro, cyano, nitro, hydroxy, trifluoromethoxy, trifluoromethyl, amino, carboxy, sulfo, carbamoyl, mercapto, sulfamoyl, methyl, ethyl, ethenyl, methoxy, ethoxy, formyl, acetyl, acetoxy, N-methylamino, N-ethylamino, N,N-dimethylamino, N,N-diethylamino, N-ethyl-N-methylamino, N-formylamino, N-acetylamino, N-methylcarbamoyl, N-ethylcarbamoyl, N,N-dimethylcarbamoyl, N,N-diethylcarbamoyl, N-ethyl-N-methylcarbamoyl, methylsulfanyl, ethylsulfanyl, methylsulfinyl, ethylsulfinyl, methylsulfonyl, methylsulfonyloxy, ethylsulfonyl, ethylsulfonyloxy, methoxycarbonyl, ethoxycarbonyl, N-methylsulfamoyl, N-ethylsulfamoyl, N,N-dimethylsulfamoyl, N,N-diethylsulfamoyl and N-ethyl-N-methylsulfamoyl;

R⁴⁹ and R⁵³ are each independently selected from C₁₋₆alkyl, C₃₋₆cycloalkyl, C₁₋₆alkanoyl, C₁₋₆alkylsulfonyl, C₁₋₆alkoxycarbonyl, carbamoyl, N—(C₁₋₆alkyl)carbamoyl, N,N—(C₁₋₆alkyl)₂carbamoyl, benzyl, benzyloxycarbonyl, benzoyl and phenylsulfonyl;

R¹¹, R¹², R¹³, R¹⁴, R¹⁵, R¹⁶, R¹⁷, R¹⁸, R¹⁹, R⁵⁴, R⁵⁵, R⁵⁶, R⁵⁷, R⁵⁸, R⁵⁹, R⁶⁰, R⁶¹ and R⁶² are each independently hydrogen or a group selected from C₁₋₃alkyl and cyclopropyl wherein said group may be optionally substituted on carbon by one or more R²²;

R²⁴, R²⁵, R²⁶, R²⁷, R²⁸, R²⁹, R³⁰, R³¹, R³², R⁶³, R⁶⁴, R⁶⁵, R⁶⁶, R⁶⁷, R⁶⁸, R⁶⁹, R⁷⁰ and R⁷¹ are each independently hydrogen or a group selected from C₁₋₃alkyl and cyclopropyl wherein said group may be optionally substituted on carbon by one or more R³⁵;

R³⁷, R³⁸, R³⁹, R⁴⁰, R⁴¹, R⁴², R⁴³, R⁴⁴, R⁴⁵, R⁷², R⁷³, R⁷⁴, R⁷⁵, R⁷⁶, R⁷⁷, R⁷⁸, R⁷⁹ and R⁸⁰ are each independently hydrogen or a group selected from C₁₋₃alkyl and cyclopropyl wherein said group may be optionally substituted on carbon by one or more R⁴⁸;

R⁸¹ and R⁸² are each independently hydrogen or a group selected from C₁₋₃alkyl and cyclopropyl wherein said group may be optionally substituted on carbon by one or more R⁵²;

or a pharmaceutically acceptable salt thereof.

A “heterocyclyl” is a saturated, partially saturated or fully unsaturated, mono or bicyclic ring system containing 4-12 ring atoms of which 1 to 4 ring atoms are chosen from nitrogen, sulfur or oxygen, which unless otherwise specified may be carbon or nitrogen-linked, wherein a ring —CH₂— group can optionally be replaced by a —C(O)—, a ring sulfur may be optionally oxidised to form the S-oxides and a ring nitrogen may be optionally oxidised to form the N-oxide.

In one aspect of the invention a “heterocyclyl” is a saturated, partially saturated or fully unsaturated, mono or bicyclic ring system containing 5-9 ring atoms of which 1 or 2 ring atoms are chosen from nitrogen, sulfur or oxygen, which unless otherwise specified may be carbon or nitrogen-linked, wherein a ring —CH₂— group can optionally be replaced by a —C(O)—, a ring sulfur may be optionally oxidised to form the S-oxides and a ring nitrogen may be optionally oxidised to form the N-oxide.

Bicyclic ring systems include fused ring systems, and bridged ring systems. One example of a bridged bicyclic ring system is a 9-azabicyclo[3.3.1]nonyl bicylyic ring system.

In another aspect of the invention a “heterocyclyl” is a saturated mono or bicyclic ring system containing 5-9 ring atoms of which 1 or 2 ring atoms are chosen from nitrogen, sulfur or oxygen, which unless otherwise specified may be carbon or nitrogen-linked and a ring sulfur may be optionally oxidised to form the S-oxides.

Examples of heterocyclyl include morpholinyl, piperidinyl, pyridyl, pyranyl, pyrrolyl, pyrazolyl, isothiazolyl, indolyl, indolinyl, benzo[b]furanyl, 1,1-dioxido-1,2,5-thiadiazolidin-3-yl, 1H-indazolyl, benzimidazolyl, benzthiazolyl, isoquinolinyl, cinnolinyl, quinoxalinyl, 1,8-naphthyridinyl, pteridinyl, quinolyl, isoquinolyl, 1H-pyrrolo[2,3-b]pyridinyl, thienyl, furyl, 1,3-benzodioxolyl, thiadiazolyl, piperazinyl, thiazolidinyl, pyrrolidinyl, thiomorpholinyl, pyrrolinyl, 9-azabicyclo[3.3.1]nonyl, 1,4-diazepan-1-yl, 3,5-dioxapiperidinyl, tetrahydropyranyl, imidazolyl, pyrimidyl, pyrazinyl, pyridazinyl, isoxazolyl, 4-pyridone, 1-isoquinolone, 2-pyrrolidone and 4-thiazolidone.

Further examples of heterocyclyl are 1-piperazinyl, piperidin-4-yl, pyrrolidin-3-yl, 9-azabicyclo[3.3.1]non-3-yl, piperazin-1-yl, morpholin-4-yl, pyrrolidin-1-yl, 1,1-dioxidothiomorpholin-4-yl, 1,4-diazepan-1-yl and 1-piperidinyl.

In one embodiment a heterocyclyl is selected from piperidinyl, piperazinyl, morpholinyl, pyrrolidinyl, 1,1-dioxido-1,4-thiazinanyl, 9-azabicyclo[3.3.1]nonyl, 1,4-diazepanyl, imidazolyl, 1,3-oxazolyl and pyrazolyl.

A “carbocyclyl” is a saturated, partially saturated or fully unsaturated, mono or bicyclic ring containing 3-12 ring atoms; wherein a —CH₂— group can optionally be replaced by a —C(O)—.

Examples of “carbocyclyl” include cyclopropyl, cyclobutyl, 1-oxocyclopentenyl, cyclopentyl, cyclopentenyl, cyclohexyl, cyclohexenyl, phenyl, naphthyl, tetralinyl, indanyl and 1-oxoindanyl.

A “C₄₋₁₂-carbocyclyl” is a saturated, partially saturated or fully unsaturated, mono or bicyclic ring containing 4-12 ring atoms; wherein a —CH₂— group can optionally be replaced by a —C(O)—.

In one embodiment a carbocyclyl is phenyl.

Where a group may be optionally substituted by “one or more” R^{x}, it is to be understood that the selection is to be made from all of the substituents listed for R^{x} and that when two or more substituents are chosen these may be the same or different.

The term “halo” refers to fluoro, chloro, bromo and iodo.

In this specification, the term “alkyl” includes both straight and branched chain alkyl groups.

References to individual alkyl groups such as “propyl” are specific for the straight chain version only and references to individual branched chain alkyl groups such as “isopropyl” are specific for the branched chain version only. This convention applies to other radicals described within this specification such as alkenyl radicals, alkynyl radicals, alkoxy radicals and alkanoyl radicals.

For example, “C₁₋₆alkyl” includes C₁₋₄alkyl, C₁₋₃alkyl, methyl, ethyl, propyl, isopropyl and t-butyl.

Examples of “C₁₋₃alkyl” are methyl, ethyl, propyl and isopropyl.

In this specification “C₂₋₆alkenyl” includes C₂₋₃alkenyl, butenyl, isobutenyl, 1,5-hexadien-3-yl. Examples of “C₂₋₃alkenyl” are ethenyl, prop-2-en-1-yl and prop-1-en-2-yl.

Examples of the term “C₂₋₆alkynyl” include C₂₋₃alkynyl, butynyl, propynyl and ethynyl.

Examples of the term “C₁₋₆alkoxy” include C₁₋₃alkoxy, t-butyloxy, isopropoxy, butoxy, ethoxy and methoxy.

Examples of the term “(C₁₋₆alkyl)-S(O)ₐ— wherein a is 0 to 2” include “(C₁₋₆alkyl)-S—”, “(C₁₋₃alkyl)-S(O)ₐ— wherein a is 0 to 2”, “(C₁₋₃alkyl)-S(O)₂—”, isopropylsulfanyl, propylsulfonyl, mesyl and ethylsulfanyl, butanesulfinyl and isopentylsulfinyl.

Examples of the term “C₁₋₆alkoxycarbonyl” include C₁₋₃alkoxycarbonyl, methoxycarbonyl, ethoxycarbonyl, isopropoxycarbonyl and isopentoxycarbonyl.

Examples of the term “C₁₋₆alkylsulfonyl” include C₁₋₃alkylsulfonyl, mesyl, ethylsulfonyl, isopropylsulfonyl and isobutylsulfonyl.

Examples of the term “C₁₋₆alkylsulfinyl” include C₁₋₃alkylsulfinyl, methylsulfinyl, ethylsulfinyl, isopropylsulfanyl and isobutylsulfinyl.

Examples of the term “C₁₋₆alkylsulfanyl” include C₁₋₃alkylsulfanyl, methylsulfanyl, ethylsulfanyl, isopropylsulfanyl and isobutylsulfanyl.

Examples of the term “C₁₋₆alkylsulfonyloxy” include C₁₋₃alkylsulfonyloxy, mesyloxy, ethylsulfonyloxy, isopropylsulfonyloxy and isobutylsulfonyloxy.

Examples of the term “N—(C₁₋₆alkyl)sulfamoyloxy” include N—(C₁₋₃alkyl)sulfamoyloxy, N-(t-butyl)sulfamoyloxy, N-(hex-3-yl)sulfamoyloxy, and N-ethylsulfamoyloxy.

Examples of the term “N,N—(C₁₋₆alkyl)₂sulfamoyloxy” include N,N—(C₁₋₃alkyl)₂sulfamoyloxy, N-(t-butyl)-N-(ethyl)sulfamoyloxy and N,N-diethylsulfamoyloxy.

Examples of the term “C₁₋₆alkanoyl” include C₁₋₃alkanoyl, formyl, acetyl and propionyl.

Examples of the term “C₁₋₆alkanoyloxy” include C₁₋₃alkanoyloxy, acetyloxy and propionyloxy.

Examples of the term “N—(C₁₋₆alkyl)amino” include N—(C₁₋₃alkyl)amino, methylamino, isopropylamino and isohexylamino.

Examples of the term “N,N—(C₁₋₆alkyl)₂amino” include N,N—(C₁₋₃alkyl)₂amino, N,N-dimethylamino, N-isopropyl-N-methylamino and N-pentyl-N-ethylamino.

Examples of the term “N—(C₁₋₆alkanoyl)-N—(Rⁿ)amino” wherein Rⁿ can be hydrogen, C₁₋₃alkyl or cyclopropyl, include N—(C₁₋₃alkanoyl)-N—(Rⁿ)amino, N-propionoyl-N—(Rⁿ)amino, N-propionoylamino, N-acetyl-N-methylamino and N-acetyl-N-cyclopropylamino.

Examples of the term “N—(C₁₋₆alkoxycarbonyl)-N—(Rⁿ)amino” wherein Rⁿ can be hydrogen, C₁₋₃ alkyl or cyclopropyl, include N—(C₁₋₃alkoxycarbonyl)-N—(Rⁿ)amino, N—(C₁₋₆alkoxycarbonyl)-N-amino, N-isopentoxycarbonyl-N-ethylamino, N-propoxycarbonyl-N-cyclopropylamino and N-methoxycarbonylamino.

Examples of “N—(C₁₋₆alkyl)carbamoyl” include N—(C₁₋₃alkyl)carbamoyl, N-isopentylaminocarbonyl, N-methylaminocarbonyl and N-ethylaminocarbonyl.

Examples of “N,N—(C₁₋₆alkyl)₂carbamoyl” include N,N—(C₁₋₃alkyl)₂carbamoyl, N-isopentyl-N-ethylaminocarbonyl, N,N-dimethylaminocarbonyl and N-methyl-N-ethylaminocarbonyl.

Examples of “N—(C₁₋₆alkyl)sulfamoyl” include N—(C₁₋₃alkyl)sulfamoyl, N-isopentylsulfamoyl, N-methylsulfamoyl and N-ethylsulfamoyl.

Examples of “N,N—(C₁₋₆alkyl)₂sulfamoyl” include N,N—(C₁₋₃alkyl)₂sulfamoyl, N-isopentyl-N-ethylsulfamoyl, N,N-dimethylsulfamoyl and N-methyl-N-ethylsulfamoyl.

Examples of “N—[(C₁₋₆alkyl)sulfonyl]-N—(Rⁿ)amino” wherein Rⁿ can be hydrogen, C₁₋₃alkyl or cyclopropyl, include N—[(C₁₋₃alkyl)sulfonyl]-N—(Rⁿ)amino, N—[(C₁₋₆alkyl)sulfonyl]amino, N-(isopentylsulfonyl)-N-(cyclopropyl)amino, N-mesyl-N-ethylamino and N-(isopropylsulfonyl)amino.

Examples of the term “(N,N—(Rⁿ)(R^{m})sulfamoyl)-N—(R^{q})amino” wherein Rⁿ, R^{m}, and R^{q} can each represent hydrogen, C₁₋₃alkyl or cyclopropyl, include (N-ethyl-N-methylsulfamoyl)amino, (sulfamoyl)-N-cyclopropylamino and (N,N-dimethylsulfamoyl)-N-isopropylamino.

Examples of “3,3-(Rⁿ)(R^{m})-1-(R^{q})ureido” wherein Rⁿ, R^{m}, and R^{q} can each represent hydrogen, C₁₋₃ alkyl or cyclopropyl, include 3-propyl-1-methylureido, 3,3-dimethylureido, 1-cyclopropylureido, 3-cyclopropyl-3-methyl-1-ethylureido and ureido.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, citric or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or triethanolamine.

Some compounds of the formula (I) may have chiral centres and/or geometric isomeric centres (E- and Z-isomers), and it is to be understood that the invention encompasses all such optical, diastereoisomers and geometric isomers that possess TTK inhibitory activity. The invention further relates to any and all tautomeric forms of the compounds of the formula (I) that possess TTK inhibitory activity.

It is also to be understood that certain compounds of the formula (I) can exist in solvated as well as unsolvated forms such as, for example, hydrated forms. It is to be understood that the invention encompasses all such solvated forms which possess TTK inhibitory activity.

Some values of variable groups are as follows. Such values may be used where appropriate with any of the definitions, claims or embodiments defined hereinbefore or hereinafter.

In one embodiment R¹ is selected from C₁₋₄alkyl, cyclopropyl, cyclopropylmethyl and cyclobutyl; wherein R¹ may be optionally substituted by one or more R⁵; and

R⁵ is cyano or fluoro.

In one embodiment R¹ is C₁₋₄alkyl wherein R¹ may be optionally substituted by one or more R⁵; and R⁵ is cyano.

In a further embodiment R¹ is methyl or ethyl wherein R¹ may be optionally substituted by one or more R⁵; and R⁵ is cyano.

In a further embodiment R¹ is selected from methyl, ethyl and cyanomethyl.

In a further embodiment m is 0.

In one embodiment R² is selected from C₁₋₆alkyl, C₂₋₆alkenyl, C₂₋₆alknyl, C₃₋₆cycloalkyl, cyclopentenyl, cyclohexenyl, oxetanyl, tetrahydropyranyl, tetrahydrofuranyl, oxepanyl, azetidinyl, pyrrolidinyl, piperidinyl and azepanyl.

In a further embodiment R² is C₁₋₆alkyl, C₃₋₆cycloalkyl or piperidinyl.

In a further embodiment R² is isopropyl, cyclopentyl or piperidin-4-yl.

In a further embodiment R² is C₁₋₆alkyl or C₃₋₆cycloalkyl.

In a further embodiment R² is isopropyl or cyclopentyl.

In one embodiment R² is C₃₋₆cycloalkyl.

In a further embodiment R² is C₁₋₆alkyl.

In a further embodiment R² is isopropyl.

In a further embodiment R² is cyclopentyl.

In one embodiment R³ is independently selected from fluoro, chloro, cyano, methoxy, ethoxy, trifluoromethoxy, methyl, ethyl and trifluoromethyl.

In one embodiment R³ is independently selected from fluoro, chloro, methoxy, ethoxy and methyl.

In a further embodiment R³ is selected from methoxy and ethoxy.

In a further embodiment R³ is methoxy.

In one embodiment n is an integer selected from 0 to 2; wherein the values of R³ may be the same or different.

In a further embodiment n is 1 or 2, wherein the values of R³ may be the same or different.

In a further embodiment n is 2; wherein the values of R³ may be the same or different.

In a further embodiment n is 1.

In a further embodiment n is 1 and R³ is methoxy.

In one embodiment n is 0.

In a further embodiment:

R⁴ is -L-R⁸ or R⁹;

L is —X—C₁₋₂alkylene—wherein X is a direct bond, —O—, —S—, —NH—, —OSO₂—, —N(CH₃)— or —N(CH₂R¹⁰)—;

R⁸ and R¹⁰ are each independently selected from hydroxy, N—(C₁₋₆alkyl)amino, N,N—(C₁₋₆alkyl)₂amino, carbocyclyl-R²⁰— and heterocyclyl-R²¹—; wherein if said heterocyclyl has an —NH— moiety, that nitrogen may be optionally substituted by R²³;

R⁹ is selected from carboxy, carbamoyl, sulfamoyl, C₃₋₆alkyl, C₃₋₆alkenyl, C₃₋₆alkynyl, C₃₋₆alkoxy, C₁₋₆alkylsulfonyl, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—[(C₁₋₆alkyl)sulfonyl]amino, C₄₋₁₂carbocyclyl-R³³— and heterocyclyl-R³⁴—; wherein R⁹ may be optionally substituted on carbon by one or more R³⁵, and wherein if said heterocyclyl has an —NH— moiety, that nitrogen may be optionally substituted by R³⁶;

R³⁵ are independently selected from N—(C₁₋₆alkyl)amino, N,N—(C₁₋₆alkyl)₂amino, N—(C₁₋₆alkanoyl)amino, N—[(C₁₋₆alkyl)sulfonyl]amino and heterocyclyl-R⁴⁷—; wherein R³⁵ may be optionally substituted on carbon by one or more R⁴⁸ and wherein if said heterocyclyl has an —NH— moiety, that nitrogen may be optionally substituted by R⁴⁹;

R²³ and R³⁶ are independently selected from C₁₋₆alkyl and heterocyclyl-R⁵¹—wherein R²³ and R³⁶ may be independently optionally substituted on carbon by one or more R⁵²; and wherein if said heterocyclyl contains an —NH— moiety, that nitrogen may be optionally substituted by R⁵³;

R²⁰, R²¹, R³³, R³⁴, R⁴⁷ and R⁵¹ are each independently selected from a direct bond, —O—, —NH—. —C(O)—, —NH—C(O)— and —SO₂—;

R⁴⁸ and R⁵² are each independently selected from fluoro, chloro, cyano, hydroxy, trifluoromethoxy, trifluoromethyl, amino, carbamoyl, sulfamoyl, methyl, ethyl, methoxy, ethoxy, formyl, acetyl, acetoxy, N-methylamino and N,N-dimethylamino; and

R⁴⁹ and R⁵³ are each independently C₁₋₆alkyl.

In one embodiment R⁴ is -L-R⁸ or R⁹;

L is —X—C₁₋₂alkylene—wherein X is a direct bond or —O—;

R⁸ is N,N—(C₁₋₆alkyl)₂amino, carbocyclyl or heterocyclyl and wherein if said heterocyclyl has an —NH— moiety, that nitrogen may be optionally substituted by R²³;

R⁹ is selected from carboxy, sulfamoyl, C₃₋₆alkoxy, N—(C₁₋₆alkyl)carbamoyl, N,N—(C₁₋₆alkyl)₂carbamoyl, N—[(C₁₋₆alkyl)sulfonyl]amino, C₄₋₁₂carbocyclyl-R³³— and heterocyclyl-R³⁴—, wherein R⁹ may be optionally substituted on carbon by one or more R³⁵, and wherein if said heterocyclyl has an —NH— moiety, that nitrogen may be optionally substituted by R³⁶;

R³⁵ are independently selected from N,N—(C₁₋₆alkyl)₂amino and heterocyclyl; wherein if said heterocyclyl has an —NH— moiety, that nitrogen may be optionally substituted by R⁴⁹;

R²³ and R³⁶ are independently selected from C₁₋₆alkyl and heterocyclyl wherein R²³ and R³⁶ may be independently optionally substituted on carbon by one or more R⁵²; and wherein if said heterocyclyl contains an —NH— moiety, that nitrogen may be optionally substituted by R⁵³;

R³³ and R³⁴ are each independently selected from a direct bond, —O—, —NH—. —C(O)—, —NH—C(O)— and —SO₂—;

R⁵² is methoxy; and

R⁴⁹ and R⁵³ are each independently C₁₋₆alkyl.

In one embodiment R⁴ is -L-R⁸ or R⁹;

L is —X—C₁₋₂alkylene—wherein X is a direct bond or —O—;

R⁸ is N,N—(C₁₋₆alkyl)₂amino, phenyl or piperazinyl and wherein said piperazinyl may be optionally substituted on nitrogen by R²³;

R⁹ is selected from carboxy, sulfamoyl, C₃₋₆alkoxy, C₁₋₆alkylsulfonyl, N—(C₁₋₆alkyl)carbamoyl, N,N—(C₁₋₆alkyl)₂carbamoyl, N—[(C₁₋₆alkyl)sulfonyl]amino, cyclohexyl-R³³—, phenyl-R³³— and a heterocyclyl-R³⁴—; wherein said heterocyclyl is selected from piperidinyl, piperazinyl, morpholinyl, pyrrolidinyl, 1,1-dioxo-1,4-thiazinanyl, 9-azobicyclo[3.3.1]nonyl, 1,4-diazepanyl, oxazolyl and pyrazolyl; wherein R⁹ may be optionally substituted on carbon by one or more R³⁵, and wherein if said heterocyclyl has an —NH— moiety, that nitrogen may be optionally substituted by R³⁶;

R³⁵ is N,N—(C₁₋₆alkyl)₂amino or a heterocyclyl selected from imidazolyl, pyrrolidinyl, piperidinyl and piperazinyl; wherein if said heterocyclyl has an —NH— moiety, that nitrogen may be optionally substituted by R⁴⁹;

R²³ and R³⁶ are independently selected from C₁₋₆alkyl and piperidinyl wherein R²³ and R³⁶ may be independently optionally substituted on carbon by one or more R⁵²; and wherein if said piperidinyl contains an —NH— moiety, that nitrogen may be optionally substituted by R⁵³;

R³³ and R³⁴ are each independently selected from a direct bond, —O—, —NH—, —C(O)—, —NH—C(O)— and —SO₂—;

R⁵² is methoxy; and

R⁴⁹ and R⁵³ are each independently C₁₋₆alkyl.

In one embodiment R⁴ is -L-R⁸ or R⁹;

L is —X—C₁₋₂alkylene—wherein X is a direct bond or —O—;

R⁸ is dimethylamino, phenyl or piperazinyl and wherein said piperazinyl may be optionally substituted on nitrogen by R²³;

R⁹ is selected from carboxy, sulfamoyl, isopropoxy, mesyl, methylcarbamoyl, ethylcarbamoyl, propylcarbamoyl, butylcarbamoyl, dimethylcarbamoyl, N-methyl-N-propylcarbamoyl, mesylamino, cyclohexyl-R³³—, phenyl-R³³— and a heterocyclyl-R³⁴—; wherein said heterocyclyl is selected from piperidinyl, piperazinyl, morpholinyl, pyrrolidinyl, 1,1-dioxo-1,4-thiazinanyl, 9-azobicyclo[3.3.1]nonyl, 1,4-diazepanyl, oxazolyl and pyrazolyl; wherein R⁹ may be optionally substituted on carbon by one or more R³⁵, and wherein if said heterocyclyl has an —NH— moiety, that nitrogen may be optionally substituted by R³⁶;

R³⁵ is dimethylamino or a heterocyclyl selected from imidazolyl, pyrrolidinyl, piperidinyl and piperazinyl; wherein if said heterocyclyl has an —NH— moiety, that nitrogen may be optionally substituted by R⁴⁹;

R²³ and R³⁶ are independently selected from methyl, ethyl and piperidinyl wherein R²³ and R³⁶ may be independently optionally substituted on carbon by one or more R⁵²; and wherein if said piperidinyl contains an —NH— moiety, that nitrogen may be optionally substituted by R⁵³;

R³³ and R³⁴ are each independently selected from a direct bond, —O—, —NH—, —C(O)—, —NH—C(O)— and —SO₂—;

R⁵² is methoxy; and

R⁴⁹ and R⁵³ are each independently methyl, ethyl or isopropyl.

In one embodiment:

R⁴ is -L-R⁸ or R⁹;

L is —X—C₁₋₂alkylene—wherein X is a direct bond or —O—;

R⁸ is N,N—(C₁₋₆alkyl)₂amino or heterocyclyl and wherein if said heterocyclyl has an —NH— moiety, that nitrogen may be optionally substituted by R²³;

R⁹ is selected from sulfamoyl, C₁₋₆alkylsulfonyl, N—(C₁₋₆alkyl)carbamoyl, N—[(C₁₋₆alkyl)sulfonyl]amino, and heterocyclyl-R³⁴—, wherein R⁹ may be optionally substituted on carbon by one or more R³⁵, and wherein if said heterocyclyl has an —NH— moiety, that nitrogen may be optionally substituted by R³⁶;

R²³ and R³⁶ are each independently C₁₋₆alkyl;

R³⁴ is a direct bond, —O—, —NH—, —NHC(O)—, —C(O)— or —S(O)₂—; and

R³⁵ is N,N—(C₁₋₆alkyl)₂amino.

In a further embodiment R⁴ is -L-R⁸ or R⁹;

L is —O—CH₂CH₂— or —CH₂CH₂—;

R⁸ is dimethylamino or 1-piperazinyl wherein the —NH— moiety of the 1-piperazinyl may be optionally substituted by R²³;

R⁹ is selected from sulfamoyl, mesyl, N-(methyl)carbamoyl, N-(mesyl)amino, piperidin-4-yl-R³⁴—, pyrrolidin-3-yl-R³⁴—, 9-azabicyclo[3.3.1]non-3-yl-R³⁴—, piperazin-1-yl-R³⁴—, morpholin-4-yl-R³⁴—, pyrrolidin-1-yl-R³⁴—, 1,1-dioxidothiomorpholin-4-yl-R³⁴—, 1,4-diazepan-1-yl-R³⁴— and 1-piperidinyl-R³⁴—; wherein R⁹ may be optionally substituted on carbon by one or more R³⁵, and wherein if said heterocyclyl has an —NH— moiety, that nitrogen may be optionally substituted by R³⁶;

R²³ is methyl;

R³⁴ is a direct bond, —O—, —NH—, —NHC(O)—, C(O)— or —S(O)₂—;

R³⁵ is N,N-dimethylamino; and

R³⁶ is methyl or ethyl.

In a further embodiment:

R⁴ is -L-R⁸ or R⁹;

L is —O—CH₂CH₂— or —CH₂CH₂—;

R⁸ is dimethylamino or 1-piperazinyl wherein the —NH— moiety of the 1-piperazinyl may be optionally substituted by R²³;

R⁹ is selected from sulfamoyl, mesyl, N-(methyl)carbamoyl, N-(mesyl)amino and a heterocyclyl-R³⁴— selected from piperidin-4-yl-R³⁴—, pyrrolidin-3-yl-R³⁴—, 9-azabicyclo[3.3.1]non-3-yl-R³⁴—, piperazin-1-yl-R³⁴—, morpholin-4-yl-R³⁴—, pyrrolidin-1-yl-R³⁴—, 1,1-dioxidothiomorpholin-4-yl-R³⁴—, 1,4-diazepan-1-yl-R³⁴— and 1-piperidinyl-R³⁴—; wherein R⁹ may be optionally substituted on carbon by one or more R³⁵, and wherein if said heterocyclyl has an —NH— moiety, that nitrogen may be optionally substituted by R³⁶;

R²³ is methyl;

R³⁴ is a direct bond, —O—, —NH—, —NHC(O)—, C(O)— or —S(O)₂—;

R³⁵ is N,N-dimethylamino; and

R³⁶ is methyl or ethyl.

In a further embodiment R⁴ is selected from: N-(1-methylpiperidin-4-yl)carbamoyl, sulfamoyl, mesyl, 4-methylpiperazin-1-yl, 1-methylpiperidin-4-yloxy, morpholin-4-yl, mesylamino, pyrrolidin-1-ylcarbonyl, N-(1-methylpiperidin-4-yl)carbamoyl, methylcarbamoyl, 1,1-dioxo-1,4-thiazinan-4-yl, (4-methylpiperazin-1-yl)sulfonyl, [(9-methyl-9-azabicyclo[3.3.1]non-3-yl)amino]carbonyl, N-(1-ethylpiperidin-4-yl)carbamoyl, 4-methyl-1,4-diazepan-1-yl, 2-hydroxyethyl, 1-methylpiperidin-4-ylamino, 4-(dimethylamino)piperidin1-yl, piperidin-1-yl, benzyl (1-methylpyrrolidin-3-yl)oxy, 2-(dimethylamino)ethoxy, 2-(4-methylpiperazin-1-yl)ethyl, 1-methylpiperidin-4-yl, 4-ethylpiperazin-1-yl, carboxy, (4-methylpiperazin-1-yl)carbonyl, 4-(1-methylpiperidin-4-yl)piperazin-1-ylcarbonyl, 3-(imidazol-1-yl)propylcarbamoyl, N-methyl-N-[(1-isopropylpyrrolidin-3-yl)methyl]-carbamoyl, dimethylcarbamoyl, N-methyl-N-(3-dimethylaminopropyl)carbamoyl, benzoyl, isopropoxy, phenoxy, 3-(dimethylamino)pyrrolidin-1-ylcarbonyl, 4-(pyrrolidin-1-yl)piperidin-1-ylcarbonyl, 4-(2-methoxyethyl)piperazin-1-ylcarbonyl, (4-dimethylaminocyclohexyl)carbamoyl, [1-(2-methoxyethyl)piperidin-4-yl]carbamoyl, pyrrolidin-3-ylcarbamoyl, oxazol-5-yl, N-[1-ethylpyrrolidin-2-yl)methyl]carbamoyl, N-[4-(dimethylamino)butyl]carbamoyl, N-[3-(dimethylamino)propyl]carbamoyl, N-[2-(piperidin-1-yl)ethyl]carbamoyl, N-[2-(4-methylpiperazin-1-yl)ethyl]carbamoyl, N-[4-(pyrrolidin-1-yl)butyl]carbamoyl, N-[2-(dimethylamino)ethyl]carbamoyl and pyrazol-1-yl.

In a further embodiment R⁴ is selected from (1-methylpiperidin-4-yl)carbamoyl, (1-ethylpiperidin-4-yl)carbamoyl, sulfamoyl, mesyl, 4-methyl-piperazin-1-yl, (1-methylpiperidin-4-yl)oxy, morpholin-4-yl, mesylamino, N-methylcarbamoyl, pyrrolidin-1-ylcarbonyl, 1,1-dioxothiomorpholin-4-yl, (4-methylpiperazin-1-yl)sulfonyl, 2-(4-methylpiperazin-1-yl)ethyl, 4-methyl-1,4-diazepan-1-yl, (1-methylpiperidin-4-yl)amino, 4-(dimethylamino)piperidin-1-yl, (1-methylpyrrolidin-3-yl)oxy, 2-(N,N-dimethylamino)ethoxy, 1-methylpiperidin-4-yl and (9-methyl-9-azabicyclo[3.3.1]non-3-yl)carbamoyl.

In a further embodiment R⁴ is selected from (1-methylpiperidin-4-yl)carbamoyl, (1-ethylpiperidin-4-yl)carbamoyl, sulfamoyl, mesyl, 4-methyl-piperazin-1-yl, (1-methylpiperidin-4-yl)oxy, morpholin-4-yl, mesylamino, N-methylcarbamoyl, pyrrolidin-1-ylcarbonyl, 1,1-dioxothiomorpholin-4-yl, (4-methylpiperazin-1-yl)sulfonyl, 2-(4-methylpiperazin-1-yl)ethyl, 4-methyl-1,4-diazepan-1-yl, (1-methylpiperidin-4-yl)amino, 4-(dimethylamino)piperidin-1-yl, (1-methylpyrrolidin-3-yl)oxy, 2-(N,N-dimethylamino)ethoxy and (9-methyl-9-azabicyclo[3.3.1]non-3-yl)carbamoyl.

Therefore in one aspect there is provided a compound of formula (I), as depicted above, wherein:

R¹ is C₁₋₄alkyl wherein R¹ may be optionally substituted by one or more R⁵; and R⁵ is cyano;

R² is selected from C₁₋₆alkyl, C₂₋₆alkenyl, C₂₋₆alknyl, C₃₋₆cycloalkyl, cyclopentenyl, cyclohexenyl, oxetanyl, tetrahydropyranyl, tetrahydrofuranyl, oxepanyl, azetidinyl, pyrrolidinyl, piperidinyl and azepanyl;

m is 0;

R³ is independently selected from fluoro, chloro, cyano, methoxy, ethoxy, trifluoromethoxy, methyl, ethyl and trifluoromethyl;

n is an integer selected from 0 to 2; wherein the values of R³ may be the same or different;

R⁴ is -L-R⁸ or R⁹;

L is —X—C₁₋₂alkylene—wherein X is a direct bond, —O—, —S—, —NH—, —OSO₂—, —N(CH₃)— or —N(CH₂R¹⁰)—;

R⁸ and R¹⁰ are each independently selected from hydroxy, N—(C₁₋₆alkyl)amino, N,N—(C₁₋₆alkyl)₂amino, carbocyclyl-R²⁰— and heterocyclyl-R²¹—; wherein if said heterocyclyl has an —NH— moiety, that nitrogen may be optionally substituted by R²³;

R⁹ is selected from carboxy, carbamoyl, sulfamoyl, C₃₋₆alkyl, C₃₋₆alkenyl, C₃₋₆alkynyl, C₃₋₆alkoxy, C₁₋₆alkylsulfonyl, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—[(C₁₋₆alkyl)sulfonyl]amino, C₄₋₁₂carbocyclyl-R³³— and heterocyclyl-R³⁴—; wherein R⁹ may be optionally substituted on carbon by one or more R³⁵, and wherein if said heterocyclyl has an —NH— moiety, that nitrogen may be optionally substituted by R³⁶;

R³⁵ are independently selected from N—(C₁₋₆alkyl)amino, N,N—(C₁₋₆alkyl)₂amino, N—(C₁₋₆alkanoyl)amino, N—[(C₁₋₆alkyl)sulfonyl]amino and heterocyclyl-R⁴⁷—; wherein R³⁵ may be optionally substituted on carbon by one or more R⁴⁸ and wherein if said heterocyclyl has an —NH— moiety, that nitrogen may be optionally substituted by R⁴⁹;

R²³ and R³⁶ are independently selected from C₁₋₆alkyl and heterocyclyl-R⁵¹—wherein R²³ and R³⁶ may be independently optionally substituted on carbon by one or more R⁵²; and wherein if said heterocyclyl contains an —NH— moiety, that nitrogen may be optionally substituted by R⁵³;

R²⁰, R²¹, R³³, R³⁴, R⁴⁷ and R⁵¹ are each independently selected from a direct bond, —O—, —NH—, —C(O)—, —NH—C(O)— and —SO₂—;

R⁴⁸ and R⁵² are each independently selected from fluoro, chloro, cyano, hydroxy, trifluoromethoxy, trifluoromethyl, amino, carbamoyl, sulfamoyl, methyl, ethyl, methoxy, ethoxy, formyl, acetyl, acetoxy, N-methylamino and N,N-dimethylamino; and

R⁴⁹ and R⁵³ are each independently C₁₋₆alkyl;

or a pharmaceutically acceptable salt thereof;
wherein the compound of formula (I) is other than:
2-{[4-(4-acetylpiperazin-1-yl)phenyl]amino}-7-methyl-9-pentan-3-yl-7,9-dihydro-8H-purin-8-one or
7-methyl-2-{[4-(4-methylpiperazin-1-yl)phenyl]amino}-9-pentan-3-yl-7,9-dihydro-8H-purin-8-one;
   or a pharmaceutically acceptable salt thereof.

In a further embodiment there is provided a compound of formula (I), as depicted above, wherein:

R¹ is C₁₋₄alkyl wherein R¹ may be optionally substituted by one or more R⁵; and R⁵ is cyano;

R² is C₁₋₆alkyl, C₃₋₆cycloalkyl or piperidinyl;

m is 0;

R³ is independently selected from fluoro, chloro, methoxy, ethoxy and methyl;

n is an integer selected from 0 to 2; wherein the values of R³ may be the same or different;

R⁴ is -L-R⁸ or R⁹;

L is —X—C₁₋₂alkylene—wherein X is a direct bond or —O—;

R⁸ is N,N—(C₁₋₆alkyl)₂amino, carbocyclyl or heterocyclyl and wherein if said heterocyclyl has an —NH— moiety, that nitrogen may be optionally substituted by R²³;

R⁹ is selected from carboxy, sulfamoyl, C₃₋₆alkoxy, C₁₋₆alkylsulfonyl, N—(C₁₋₆alkyl)carbamoyl, N,N—(C₁₋₆alkyl)₂carbamoyl, N—[(C₁₋₆alkyl)sulfonyl]amino, C₄₋₁₂carbocyclyl-R³³— and heterocyclyl-R³⁴—, wherein R⁹ may be optionally substituted on carbon by one or more R³⁵, and wherein if said heterocyclyl has an —NH— moiety, that nitrogen may be optionally substituted by R³⁶;

R³⁵ are independently selected from N,N—(C₁₋₆alkyl)₂amino and heterocyclyl; wherein if said heterocyclyl has an —NH— moiety, that nitrogen may be optionally substituted by R⁴⁹;

R²³ and R³⁶ are independently selected from C₁₋₆alkyl and heterocyclyl wherein R²³ and R³⁶ may be independently optionally substituted on carbon by one or more R⁵²; and wherein if said heterocyclyl contains an —NH— moiety, that nitrogen may be optionally substituted by R⁵³;

R³³ and R³⁴ are each independently selected from a direct bond, —O—, —NH—. —C(O)—, —NH—C(O)— and —SO₂—;

R⁵² is methoxy; and

R⁴⁹ and R⁵³ are each independently C₁₋₆alkyl;

or a pharmaceutically acceptable salt thereof;
wherein the compound of formula (I) is other than:
2-{[4-(4-acetylpiperazin-1-yl)phenyl]amino}-7-methyl-9-pentan-3-yl-7,9-dihydro-8H-purin-8-one or
7-methyl-2-{[4-(4-methylpiperazin-1-yl)phenyl]amino}-9-pentan-3-yl-7,9-dihydro-8H-purin-8-one;
   or a pharmaceutically acceptable salt thereof.

Therefore, in a further embodiment there is provided a compound of formula (I), as depicted above, wherein:

R¹ is C₁₋₄alkyl wherein R¹ may be optionally substituted by one or more R⁵; and R⁵ is cyano;

R² is C₁₋₆alkyl, C₃₋₆cycloalkyl or piperidinyl;

m is 0;

R³ is independently selected from fluoro, chloro, methoxy, ethoxy and methyl;

n is an integer selected from 0 to 2; wherein the values of R³ may be the same or different;

R⁴ is -L-R⁸ or R⁹;

L is —X—C₁₋₂alkylene—wherein X is a direct bond or —O—;

R⁸ is N,N—(C₁₋₆alkyl)₂amino, phenyl or piperazinyl and wherein said piperazinyl may be optionally substituted on nitrogen by R²³;

R⁹ is selected from carboxy, sulfamoyl, C₃₋₆alkoxy, C₁₋₆alkylsulfonyl, N—(C₁₋₆alkyl)carbamoyl, N,N—(C₁₋₆alkyl)₂carbamoyl, N—[(C₁₋₆alkyl)sulfonyl]amino, cyclohexyl-R³³—, phenyl-R³³— and a heterocyclyl-R³⁴—; wherein said heterocyclyl is selected from piperidinyl, piperazinyl, morpholinyl, pyrrolidinyl, 1,1-dioxo-1,4-thiazinanyl, 9-azobicyclo[3.3.1]nonyl, 1,4-diazepanyl, oxazolyl and pyrazolyl; wherein R⁹ may be optionally substituted on carbon by one or more R³⁵, and wherein if said heterocyclyl has an —NH— moiety, that nitrogen may be optionally substituted by R³⁶;

R³⁵ is N,N—(C₁₋₆alkyl)₂amino or a heterocyclyl selected from imidazolyl, pyrrolidinyl, piperidinyl and piperazinyl; wherein if said heterocyclyl has an —NH— moiety, that nitrogen may be optionally substituted by R⁴⁹;

R²³ and R³⁶ are independently selected from C₁₋₆alkyl and piperidinyl wherein R²³ and R³⁶ may be independently optionally substituted on carbon by one or more R⁵²; and wherein if said piperidinyl contains an —NH— moiety, that nitrogen may be optionally substituted by R⁵³;

R³³ and R³⁴ are each independently selected from a direct bond, —O—, —NH—. —C(O)—, —NH—C(O)— and —SO₂—;

R⁵² is methoxy; and

R⁴⁹ and R⁵³ are each independently C₁₋₆alkyl;

or a pharmaceutically acceptable salt thereof;
wherein the compound of formula (I) is other than:
2-{[4-(4-acetylpiperazin-1-yl)phenyl]amino}-7-methyl-9-pentan-3-yl-7,9-dihydro-8H-purin-8-one or
7-methyl-2-{[4-(4-methylpiperazin-1-yl)phenyl]amino}-9-pentan-3-yl-7,9-dihydro-8H-purin-8-one;
   or a pharmaceutically acceptable salt thereof.

In a further embodiment there is provided a compound of formula (I), as depicted above, wherein:

R¹ is C₁₋₄alkyl wherein R¹ may be optionally substituted by one or more R⁵; and R⁵ is cyano;

R² is isopropyl, cyclopentyl or piperidin-4-yl;

m is 0;

R³ is independently selected from fluoro, chloro, methoxy, ethoxy and methyl;

n is an integer selected from 0 to 2; wherein the values of R³ may be the same or different;

R⁴ is -L-R⁸ or R⁹;

L is —X—C₁₋₂alkylene—wherein X is a direct bond or —O—;

R⁸ is N,N—(C₁₋₆alkyl)₂amino, carbocyclyl or heterocyclyl and wherein if said heterocyclyl has an —NH— moiety, that nitrogen may be optionally substituted by R²³;

R⁹ is selected from carboxy, sulfamoyl, C₃₋₆alkoxy, C₁₋₆alkylsulfonyl, N—(C₁₋₆alkyl)carbamoyl, N,N—(C₁₋₆alkyl)₂carbamoyl, N—[(C₁₋₆alkyl)sulfonyl]amino, C₄₋₁₂carbocyclyl-R³³— and heterocyclyl-R³⁴—, wherein R⁹ may be optionally substituted on carbon by one or more R³⁵, and wherein if said heterocyclyl has an —NH— moiety, that nitrogen may be optionally substituted by R³⁶;

R³⁵ are independently selected from N,N—(C₁₋₆alkyl)₂amino and heterocyclyl; wherein if said heterocyclyl has an —NH— moiety, that nitrogen may be optionally substituted by R⁴⁹;

R²³ and R³⁶ are independently selected from C₁₋₆alkyl and heterocyclyl wherein R²³ and R³⁶ may be independently optionally substituted on carbon by one or more R⁵²; and wherein if said heterocyclyl contains an —NH— moiety, that nitrogen may be optionally substituted by R⁵³;

R³³ and R³⁴ are each independently selected from a direct bond, —O—, —NH—. —C(O)—, —NH—C(O)— and —SO₂—;

R⁵² is methoxy; and

R⁴⁹ and R⁵³ are each independently C₁₋₆alkyl;

or a pharmaceutically acceptable salt thereof.

In a further embodiment there is provided a compound of formula (I), as depicted above, wherein:

R¹ is selected from methyl, ethyl and cyanomethyl;

R² is isopropyl, cyclopentyl or piperidin-4-yl;

m is 0;

R³ is independently selected from fluoro, chloro, methoxy, ethoxy and methyl;

n is an integer selected from 0 to 2; wherein the values of R³ may be the same or different;

R⁴ is -L-R⁸ or R⁹;

L is —X—C₁₋₂alkylene—wherein X is a direct bond or —O—;

R⁸ is N,N—(C₁₋₆alkyl)₂amino, phenyl or piperazinyl and wherein said piperazinyl may be optionally substituted on nitrogen by R²³;

R⁹ is selected from carboxy, sulfamoyl, C₃₋₆alkoxy, C₁₋₆alkylsulfonyl, N—(C₁₋₆alkyl)carbamoyl, N,N—(C₁₋₆alkyl)₂carbamoyl, N—[(C₁₋₆alkyl)sulfonyl]amino, cyclohexyl-R³³—, phenyl-R³³— and a heterocyclyl-R³⁴—; wherein said heterocyclyl is selected from piperidinyl, piperazinyl, morpholinyl, pyrrolidinyl, 1,1-dioxo-1,4-thiazinanyl, 9-azobicyclo[3.3.1]nonyl, 1,4-diazepanyl, oxazolyl and pyrazolyl; wherein R⁹ may be optionally substituted on carbon by one or more R³⁵, and wherein if said heterocyclyl has an —NH— moiety, that nitrogen may be optionally substituted by R³⁶;

R³⁵ is N,N—(C₁₋₆alkyl)₂amino or a heterocyclyl selected from imidazolyl, pyrrolidinyl, piperidinyl and piperazinyl; wherein if said heterocyclyl has an —NH— moiety, that nitrogen may be optionally substituted by R⁴⁹;

R²³ and R³⁶ are independently selected from C₁₋₆alkyl and piperidinyl wherein R²³ and R³⁶ may be independently optionally substituted on carbon by one or more R⁵²; and wherein if said piperidinyl contains an —NH— moiety, that nitrogen may be optionally substituted by R⁵³;

R³³ and R³⁴ are each independently selected from a direct bond, —O—, —NH—. —C(O)—, —NH—C(O)— and —SO₂—;

R⁵² is methoxy; and

R⁴⁹ and R⁵³ are each independently C₁₋₆alkyl;

or a pharmaceutically acceptable salt thereof.

In a further embodiment there is provided a compound of formula (I), as depicted above, wherein:

R¹ is selected from methyl, ethyl and cyanomethyl;

R² is isopropyl, cyclopentyl or piperidin-4-yl;

m is 0;

R³ is independently selected from fluoro, chloro, methoxy, ethoxy and methyl;

n is an integer selected from 0 to 2; wherein the values of R³ may be the same or different;

R⁴ is selected from: N-(1-methylpiperidin-4-yl)carbamoyl, sulfamoyl, mesyl, 4-methylpiperazin-1-yl, 1-methylpiperidin-4-yloxy, morpholin-4-yl, mesylamino, pyrrolidin-1-ylcarbonyl, N-(1-methylpiperidin-4-yl)carbamoyl, methylcarbamoyl, 1,1-dioxo-1,4-thiazinan-4-yl, (4-methylpiperazin-1-yl)sulfonyl, [(9-methyl-9-azabicyclo[3.3.1]non-3-yl)amino]carbonyl, N-(1-ethylpiperidin-4-yl)carbamoyl, 4-methyl-1,4-diazepan-1-yl, 2-hydroxyethyl, 1-methylpiperidin-4-ylamino, 4-(dimethylamino)piperidin 1-yl, piperidin-1-yl, benzyl (1-methylpyrrolidin-3-yl)oxy, 2-(dimethylamino)ethoxy, 2-(4-methylpiperazin-1-yl)ethyl, 1-methylpiperidin-4-yl, 4-ethylpiperazin-1-yl, carboxy, (4-methylpiperazin-1-yl)carbonyl, 4-(1-methylpiperidin-4-yl)piperazin-1-ylcarbonyl, 3-(imidazol-1-yl)propylcarbamoyl, N-methyl-N-[(1-isopropylpyrrolidin-3-yl)methyl]-carbamoyl, dim ethyl carbamoyl, N-methyl-N-(3-dimethylaminopropyl)carbamoyl, benzoyl, isopropoxy, phenoxy, 3-(dimethylamino)pyrrolidin-1-ylcarbonyl, 4-(pyrrolidin-1-yl)piperidin-1-ylcarbonyl, 4-(2-methoxyethyl)piperazin-1-ylcarbonyl, (4-dimethylaminocyclohexyl)carbamoyl, [1-(2-methoxyethyl)piperidin-4-yl]carbamoyl, pyrrolidin-3-ylcarbamoyl, oxazol-5-yl, N-[1-ethylpyrrolidin-2-yl)methyl]carbamoyl, N-[4-(dimethylamino)butyl]carbamoyl, N-[3-(dimethylamino)propyl]carbamoyl, N-[2-(piperidin-1-yl)ethyl]carbamoyl, N-[2-(4-methylpiperazin-1-yl)ethyl]carbamoyl, N-[4-(pyrrolidin-1-yl)butyl]carbamoyl, N-[2-(dimethylamino)ethyl]carbamoyl and pyrazol-1-yl;

or a pharmaceutically acceptable salt thereof. Therefore, in a further embodiment of the invention, there is provided a compound of formula (I) wherein:

R¹ is C₁₋₄alkyl wherein R¹ may be optionally substituted by one or more R⁵;

R² is C₃₋₆cycloalkyl;

m is 0;

R³ is independently selected from fluoro, chloro, methoxy, ethoxy and methyl;

n is an integer selected from 0 to 2; wherein the values of R³ may be the same or different;

R⁴ is -L-R⁸ or R⁹;

L is —X—C₁₋₂alkylene—wherein X is a direct bond or —O—;

R⁵ is cyano;

R⁸ is N,N—(C₁₋₆alkyl)₂amino or a heterocyclyl and wherein if said heterocyclyl has an —NH— moiety, that nitrogen may be optionally substituted by R²³;

R⁹ is selected from sulfamoyl, C₁₋₆alkylsulfonyl, N—(C₁₋₆alkyl)carbamoyl, N—[(C₁₋₆alkyl)sulfonyl]amino, and heterocyclyl-R³⁴—, wherein R⁹ may be optionally substituted on carbon by one or more R³⁵, and wherein if said heterocyclyl has an —NH—moiety, that nitrogen may be optionally substituted by R³⁶;

R²³ and R³⁶ are each independently C₁₋₆alkyl;

R³⁴ is a direct bond, —O—, —NH—, —NHC(O)—, —C(O)— or —S(O)₂—; and

R³⁵ is N,N—(C₁₋₆alkyl)₂amino;

or a pharmaceutically acceptable salt thereof.

Therefore, in a further embodiment of the invention, there is provided a compound of formula (I) wherein:

R¹ is methyl or ethyl wherein R¹ may be optionally substituted by R⁵;

R² is cyclopentyl;

m is 0;

R³ is independently selected from fluoro, chloro, methoxy, ethoxy and methyl;

n is an integer selected from 0 to 2; wherein the values of R³ may be the same or different;

R⁴ is -L-R⁸ or R⁹;

L is —O—CH₂CH₂— or —CH₂CH₂—;

R⁵ is cyano;

R⁸ is dimethylamino or 1-piperazinyl wherein the —NH— moiety of the 1-piperazinyl may be optionally substituted by R²³;

R⁹ is selected from sulfamoyl, mesyl, N-(methyl)carbamoyl, N-(mesyl)amino, piperidin-4-yl-R³⁴—, pyrrolidin-3-yl-R³⁴—, and 9-azabicyclo[3.3.1]non-3-yl-R³⁴—, piperazin-1-yl-R³⁴—, morpholin-4-yl-R³⁴—, pyrrolidin-1-yl-R³⁴—, 1,1-dioxidothiomorpholin-4-yl-R³⁴—, 1,4-diazepan-1-yl-R³⁴— and 1-piperidinyl-R³⁴—;

wherein R⁹ may be optionally substituted on carbon by one or more R³⁵, and wherein if said heterocyclyl has an —NH— moiety, that nitrogen may be optionally substituted by R³⁶;

R²³ is methyl;

R³⁴ is a direct bond, —O—, —NH—, —NHC(O)—, —C(O)— or —S(O)₂—;

R³⁵ is N,N-dimethylamino; and

R³⁶ is methyl or ethyl;

or a pharmaceutically acceptable salt thereof.

Therefore, in a further embodiment of the invention, there is provided a compound of formula (I) wherein:

R¹ is methyl or ethyl wherein R¹ may be optionally substituted by R⁵;

R² is cyclopentyl;

m is 0;

R³ is independently selected from fluoro, chloro, methoxy, ethoxy and methyl;

n is an integer selected from 0 to 2; wherein the values of R³ may be the same or different;

R⁴ is -L-R⁸ or R⁹;

L is —O—CH₂CH₂— or —CH₂CH₂—;

R⁵ is cyano;

R⁸ is dimethylamino or 1-piperazinyl wherein the —NH— moiety of the 1-piperazinyl may be optionally substituted by R²³;

R⁹ is selected from sulfamoyl, mesyl, N-(methyl)carbamoyl, N-(mesyl)amino and a heterocyclyl-R³⁴— selected from piperidin-4-yl-R³⁴—, pyrrolidin-3-yl-R³⁴—, 9-azabicyclo[3.3.1]non-3-yl-R³⁴—, piperazin-1-yl-R³⁴—, morpholin-4-yl-R³⁴—, pyrrolidin-1-yl-R³⁴—, 1,1-dioxidothiomorpholin-4-yl-R³⁴—, 1,4-diazepan-1-yl-R³⁴— and 1-piperidinyl-R³⁴—; wherein R⁹ may be optionally substituted on carbon by one or more R³⁵, and wherein if said heterocyclyl has an —NH— moiety, that nitrogen may be optionally substituted by R³⁶;

R²³ is methyl;

R³⁴ is a direct bond, —O—, —NH—, —NHC(O)—, —C(O)— or —S(O)₂—;

R³⁵ is N,N-dimethylamino; and

R³⁶ is methyl or ethyl;

or a pharmaceutically acceptable salt thereof.

Therefore, in a further embodiment of the invention, there is provided a compound of formula (I) wherein:

R¹ is methyl, ethyl or cyanomethyl;

R² is cyclopentyl;

m is 0;

R³ is independently selected from fluoro, chloro, methoxy, ethoxy and methyl;

n is an integer selected from 0 to 2; wherein the values of R³ may be the same or different;

R⁴ is selected from (1-methylpiperidin-4-yl)carbamoyl, (1-ethylpiperidin-4-yl)carbamoyl, sulfamoyl, mesyl, 4-methyl-piperazin-1-yl, (1-methylpiperidin-4-yl)oxy, morpholin-4-yl, mesylamino, N-methylcarbamoyl, pyrrolidin-1-ylcarbonyl, 1,1-dioxothiomorpholin-4-yl, (4-methylpiperazin-1-yl)sulfonyl, 2-(4-methylpiperazin-1-yl)ethyl, 4-methyl-1,4-diazepan-1-yl, (1-methylpiperidin-4-yl)amino, 4-(dimethylamino)piperidin-1-yl, (1-methylpyrrolidin-3-yl)oxy, 2-(N,N-dimethylamino)ethoxy, 1-methylpiperidin-4-yl and (9-methyl-9-azabicyclo[3.3.1]non-3-yl)carbamoyl;

or a pharmaceutically acceptable salt thereof.

In a further embodiment there is a compound of formula (I) which is a compound of formula (IA):

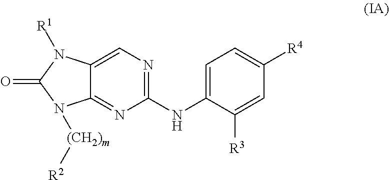

wherein:

R³ is selected from fluoro, chloro, bromo, cyano, methoxy, ethoxy, trifluoromethoxy, methyl, ethyl, trifluoromethyl, ethenyl, ethynyl, cyclopropyl, methylthio, ethylthio, N-methylamino, N,N-dimethylamino, amino and methylsulfonyloxy;

and the values of R¹, R², m and R⁴ are as described hereinbefore;

or a pharmaceutically acceptable salt thereof.

In a further embodiment there is a compound of formula (IA) wherein R³ is methoxy or ethoxy and the values of R¹, R², m, and R⁴ are as described hereinbefore. Such compounds may mediate off-target enzyme activity, for example CDK activity. CDK activity can be measured using the assay described in international patent application WO02/066481.

In a further embodiment there is a compound of formula (IA) wherein R³ is methoxy or ethoxy and R¹ is methyl, wherein the values of R², m and R⁴ are as described hereinbefore.

Therefore in a further aspect there is a compound of formula (I), which is a compound of formula (IA), as depicted above, wherein:

R³ is methoxy or ethoxy;

R¹ is C₁₋₄alkyl wherein R¹ may be optionally substituted by one or more R⁵;

R² is C₃₋₆cycloalkyl;

m is 0;

R⁴ is -L-R⁸ or R⁹;

L is —X—C₁₋₂alkylene—wherein X is a direct bond or —O—;

R⁵ is cyano;

R⁸ is N,N—(C₁₋₆alkyl)₂amino or a heterocyclyl and wherein if said heterocyclyl has an —NH— moiety, that nitrogen may be optionally substituted by R²³;

R⁹ is selected from sulfamoyl, C₁₋₆alkylsulfonyl, N—(C₁₋₆alkyl)carbamoyl, N—[(C₁₋₆alkyl)sulfonyl]amino and heterocyclyl-R³⁴—, wherein R⁹ may be optionally substituted on carbon by one or more R³⁵, and wherein if said heterocyclyl has an —NH—moiety, that nitrogen may be optionally substituted by R³⁶;

R²³ and R³⁶ are each independently C₁₋₆alkyl;

R³⁴ is a direct bond, —O—, —NH—, —NHC(O)—, —C(O)— or —S(O)₂—; and

R³⁵ is N,N—(C₁₋₆alkyl)₂amino;

or a pharmaceutically acceptable salt thereof.

In a further embodiment there is provided a compound of formula (I), which is a compound of formula (IA), as depicted above, wherein:

R³ is methoxy or ethoxy;

R¹ is C₁₋₄alkyl; wherein R¹ may be optionally substituted by one or more R⁵;

R⁵ is cyano;

R² is C₁₋₆alkyl, C₃₋₆cycloalkyl or piperidinyl;

m is 0;

R⁴ is -L-R⁸ or R⁹;

L is —X—C₁₋₂alkylene—wherein X is a direct bond or —O—;

R⁸ is N,N—(C₁₋₆alkyl)₂amino, carbocyclyl or heterocyclyl and wherein if said heterocyclyl has an —NH— moiety, that nitrogen may be optionally substituted by R²³;

R⁹ is selected from carboxy, sulfamoyl, C₃₋₆alkoxy, C₁₋₆alkylsulfonyl, N—(C₁₋₆alkyl)carbamoyl, N,N—(C₁₋₆alkyl)₂carbamoyl, N—[(C₁₋₆alkyl)sulfonyl]amino, C₄₋₁₂carbocyclyl-R³³— and heterocyclyl-R³⁴—, wherein R⁹ may be optionally substituted on carbon by one or more R³⁵, and wherein if said heterocyclyl has an —NH— moiety, that nitrogen may be optionally substituted by R³⁶;

R³⁵ are independently selected from N,N—(C₁₋₆alkyl)₂amino and heterocyclyl; wherein if said heterocyclyl has an —NH— moiety, that nitrogen may be optionally substituted by R⁴⁹;

R²³ and R³⁶ are independently selected from C₁₋₆alkyl and heterocyclyl wherein R²³ and R³⁶ may be independently optionally substituted on carbon by one or more R⁵²; and wherein if said heterocyclyl contains an —NH— moiety, that nitrogen may be optionally substituted by R⁵³;

R³³ and R³⁴ are each independently selected from a direct bond, —O—, —NH—, —C(O)—, —NH—C(O)— and —SO₂—;

R⁵² is methoxy; and

R⁴⁹ and R⁵³ are each independently C₁₋₆alkyl;

or a pharmaceutically acceptable salt thereof.

In a further embodiment there is provided a compound of formula (I), which is a compound of formula (IA), as depicted above, wherein:

R³ is methoxy or ethoxy;

R¹ is selected from methyl, ethyl and cyanomethyl;

R² is isopropyl, cyclopentyl or piperidin-4-yl;

m is 0;

R⁴ is -L-R⁸ or R⁹;

L is —X—C₁₋₂alkylene—wherein X is a direct bond or —O—;

R⁸ is N,N—(C₁₋₆alkyl)₂amino, phenyl or piperazinyl and wherein said piperazinyl may be optionally substituted on nitrogen by R²³;

R⁹ is selected from carboxy, sulfamoyl, C₃₋₆alkoxy, C₁₋₆alkylsulfonyl, N—(C₁₋₆alkyl)carbamoyl, N,N—(C₁₋₆alkyl)₂carbamoyl, N—[(C₁₋₆alkyl)sulfonyl]amino, cyclohexyl-R³³—, phenyl-R³³— and a heterocyclyl-R³⁴—; wherein said heterocyclyl is selected from piperidinyl, piperazinyl, morpholinyl, pyrrolidinyl, 1,1-dioxo-1,4-thiazinanyl, 9-azobicyclo[3.3.1]nonyl, 1,4-diazepanyl, oxazolyl and pyrazolyl; wherein R⁹ may be optionally substituted on carbon by one or more R³⁵, and wherein if said heterocyclyl has an —NH— moiety, that nitrogen may be optionally substituted by R³⁶;

R³⁵ is N,N—(C₁₋₆alkyl)₂amino or a heterocyclyl selected from imidazolyl, pyrrolidinyl, piperidinyl and piperazinyl; wherein if said heterocyclyl has an —NH— moiety, that nitrogen may be optionally substituted by R⁴⁹;

R²³ and R³⁶ are independently selected from C₁₋₆alkyl and piperidinyl wherein R²³ and R³⁶ may be independently optionally substituted on carbon by one or more R⁵²; and wherein if said piperidinyl contains an —NH— moiety, that nitrogen may be optionally substituted by R⁵³;

R³³ and R³⁴ are each independently selected from a direct bond, —O—, —NH—, —C(O)—, —NH—C(O)— and —SO₂—;

R⁵² is methoxy; and

R⁴⁹ and R⁵³ are each independently C₁₋₆alkyl;

or a pharmaceutically acceptable salt thereof.

In a further embodiment there is provided a compound of formula (I), which is a compound of formula (IA), as depicted above, wherein:

R³ is methoxy or ethoxy;

R¹ is selected from methyl, ethyl and cyanomethyl;

R² is isopropyl, cyclopentyl or piperidin-4-yl;

m is 0;

R⁴ is selected from: N-(1-methylpiperidin-4-yl)carbamoyl, sulfamoyl, mesyl, 4-methylpiperazin-1-yl, 1-methylpiperidin-4-yloxy, morpholin-4-yl, mesylamino, pyrrolidin-1-ylcarbonyl, N-(1-methylpiperidin-4-yl)carbamoyl, methylcarbamoyl, 1,1-dioxo-1,4-thiazinan-4-yl, (4-methylpiperazin-1-yl)sulfonyl, [(9-methyl-9-azabicyclo[3.3.1]non-3-yl)amino]carbonyl, N-(1-ethylpiperidin-4-yl)carbamoyl, 4-methyl-1,4-diazepan-1-yl, 2-hydroxyethyl, 1-methylpiperidin-4-ylamino, 4-(dimethylamino)piperidin1-yl, piperidin-1-yl, benzyl (1-methylpyrrolidin-3-yl)oxy, 2-(dimethylamino)ethoxy, 2-(4-methylpiperazin-1-yl)ethyl, 1-methylpiperidin-4-yl, 4-ethylpiperazin-1-yl, carboxy, (4-methylpiperazin-1-yl)carbonyl, 4-(1-methylpiperidin-4-yl)piperazin-1-ylcarbonyl, 3-(imidazol-1-yl)propylcarbamoyl, N-methyl-N-[(1-isopropylpyrrolidin-3-yl)methyl]-carbamoyl, dimethylcarbamoyl, N-methyl-N-(3-dimethylaminopropyl)carbamoyl, benzoyl, isopropoxy, phenoxy, 3-(dimethylamino)pyrrolidin-1-ylcarbonyl, 4-(pyrrolidin-1-yl)piperidin-1-ylcarbonyl, 4-(2-methoxyethyl)piperazin-1-ylcarbonyl, (4-dimethylaminocyclohexyl)carbamoyl, [1-(2-methoxyethyl)piperidin-4-yl]carbamoyl, pyrrolidin-3-ylcarbamoyl, oxazol-5-yl, N-[1-ethylpyrrolidin-2-yl)methyl]carbamoyl, N-[4-(dimethylamino)butyl]carbamoyl, N-[3-(dimethylamino)propyl]carbamoyl, N-[2-(piperidin-1-yl)ethyl]carbamoyl, N-[2-(4-methylpiperazin-1-yl)ethyl]carbamoyl, N-[4-(pyrrolidin-1-yl)butyl]carbamoyl, N-[2-(dimethylamino)ethyl]carbamoyl and pyrazol-1-yl;

or a pharmaceutically acceptable salt thereof.

In a further embodiment there is a compound of formula (I), which is a compound of formula (IA), as depicted above, wherein:

R³ is methoxy or ethoxy;

R¹ is methyl or ethyl wherein R¹ may be optionally substituted by R⁵;

R² is cyclopentyl;

m is 0;

R⁴ is -L-R⁸ or R⁹;

L is —O—CH₂CH₂— or —CH₂CH₂—;

R⁵ is cyano;

R⁸ is dimethylamino or 1-piperazinyl wherein the —NH— moiety of the 1-piperazinyl may be optionally substituted by R²³;

R⁹ is selected from sulfamoyl, mesyl, N-(methyl)carbamoyl, N-(mesyl)amino and a heterocyclyl-R³⁴— selected from piperidin-4-yl-R³⁴—, pyrrolidin-3-yl-R³⁴—, 9-azabicyclo[3.3.1]non-3-yl-R³⁴—, piperazin-1-yl-R³⁴—, morpholin-4-yl-R³⁴—, pyrrolidin-1-yl-R³⁴—, 1,1-dioxidothiomorpholin-4-yl-R³⁴—, 1,4-diazepan-1-yl-R³⁴— and 1-piperidinyl-R³⁴—; wherein R⁹ may be optionally substituted on carbon by one or more R³⁵, and wherein if said heterocyclyl has an —NH— moiety, that nitrogen may be optionally substituted by R³⁶;

R²³ is methyl;

R³⁴ is a direct bond, —O—, —NH—, —NHC(O)—, —C(O)— or —S(O)₂—;

R³⁵ is N,N-dimethylamino; and

R³⁶ is methyl or ethyl;

or a pharmaceutically acceptable salt thereof.

In a further embodiment there is provided a compound of formula (I), which is a compound of formula (IA) wherein:

R³ is methoxy or ethoxy;

R¹ is methyl, ethyl or cyanomethyl;

R² is cyclopentyl;

m is 0;

R⁴ is selected from (1-methylpiperidin-4-yl)carbamoyl, (1-ethylpiperidin-4-yl)carbamoyl, sulfamoyl, mesyl, 4-methyl-piperazin-1-yl, (1-methylpiperidin-4-yl)oxy, morpholin-4-yl, mesylamino, N-methylcarbamoyl, pyrrolidin-1-ylcarbonyl, 1,1-dioxothiomorpholin-4-yl, (4-methylpiperazin-1-yl)sulfonyl, 2-(4-methylpiperazin-1-yl)ethyl, 4-methyl-1,4-diazepan-1-yl, (1-methylpiperidin-4-yl)amino, 4-(dimethylamino)piperidin-1-yl, (1-methylpyrrolidin-3-yl)oxy, 2-(N,N-dimethylamino)ethoxy, 1-methylpiperidin-4-yl and (9-methyl-9-azabicyclo[3.3.1]non-3-yl)carbamoyl;

or a pharmaceutically acceptable salt thereof.

In another aspect of the invention, compounds of the invention are any one of the Examples or a pharmaceutically acceptable salt thereof.

In a further embodiment there is provided a compound of formula (I) selected from 4-[(9-cyclopentyl-7-methyl-8-oxo-8,9-dihydro-7H-purin-2-yl)amino]-3-methoxy-N-(1-methylpiperidin-4-yl)benzamide, 4-[(9-cyclopentyl-7-methyl-8-oxo-8,9-dihydro-7H-purin-2-yl)amino]-3-methylbenzenesulfonamide, 9-cyclopentyl-2-{[2-fluoro-4-(methylsulfonyl)phenyl]amino}-7-methyl-7,9-dihydro-8H-purin-8-one, 9-cyclopentyl-2-{[2-methoxy-4-(4-methylpiperazin-1-yl)phenyl]amino}-7-methyl-7,9-dihydro-8H-purin-8-one, 9-cyclopentyl-2-({2-methoxy-4-[(1-methylpiperidin-4-yl)oxy]phenyl}amino)-7-methyl-7,9-dihydro-8H-purin-8-one, 9-cyclopentyl-2-[(2-methoxy-4-morpholin-4-ylphenyl)amino]-7-methyl-7,9-dihydro-8H-purin-8-one, N-{4-[(9-cyclopentyl-7-methyl-8-oxo-8,9-dihydro-7H-purin-2-yl)amino]-3-methoxyphenyl}methanesulfonamide, 4-[9-cyclopentyl-7-methyl-8-oxo-8,9-dihydro-7H-purin-2-yl)amino]-2-fluoro-N-(1-methylpiperidin-4-yl)benzamide, 4-[(9-cyclopentyl-7-methyl-8-oxo-8,9-dihydro-7H-purin-2-yl)amino]-3-methoxy-N-methylbenzamide, 9-cyclopentyl-2-{[2-methoxy-4-(pyrrolidin-1-ylcarbonyl)phenyl]amino}-7-methyl-7,9-dihydro-8H-purin-8-one, 4-[(9-cyclopentyl-7-methyl-8-oxo-8,9-dihydro-7H-purin-2-yl)amino]benzenesulfonamide, 9-cyclopentyl-2-{[4-(1,1-dioxidothiomorpholin-4-yl)phenyl]amino}-7-methyl-7,9-dihydro-8H-purin-8-one, 9-cyclopentyl-7-methyl-2-({4-[(4-methylpiperazin-1-yl)sulfonyl]phenyl}amino)-7,9-dihydro-8H-purin-8-one, 4-[(9-cyclopentyl-7-methyl-8-oxo-8,9-dihydro-7H-purin-2-yl)amino]-3-methoxy-N-(9-methyl-9-azabicyclo[3.3.1]non-3-yl)benzamide, 3-chloro-4-[(9-cyclopentyl-7-methyl-8-oxo-8,9-dihydro-7H-purin-2-yl)amino]-N-(1-methylpiperidin-4-yl)benzamide, 4-[(9-cyclopentyl-7-methyl-8-oxo-8,9-dihydro-7H-purin-2-yl)amino]-3-fluoro-N-(9-methyl-9-azabicyclo[3.3.1]non-3-yl)benzamide, 4-[(9-cyclopentyl-7-methyl-8-oxo-8,9-dihydro-7H-purin-2-yl)amino]-N-(1-ethylpiperidin-4-yl)-2,5-difluorobenzamide, 2-{[2-chloro-4-(4-methylpiperazin-1-yl)phenyl]amino}-9-cyclopentyl-7-methyl-7,9-dihydro-8H-purin-8-one, 9-cyclopentyl-7-methyl-2-{[4-(4-methylpiperazin-1-yl)phenyl]amino}-7,9-dihydro-8H-purin-8-one, 9-cyclopentyl-2-{[2-methoxy-4-(4-methyl-1,4-diazepan-1-yl)phenyl]amino}-7-methyl-7,9-dihydro-8H-purin-8-one, 9-cyclopentyl-2-{[2-ethoxy-4-(4-methyl-1,4-diazepan-1-yl)phenyl]amino}-7-methyl-7,9-dihydro-8H-purin-8-one, 9-cyclopentyl-2-({2-methoxy-4-[(1-methylpiperidin-4-yl)amino]phenyl}amino)-7-methyl-7,9-dihydro-8H-purin-8-one, 9-cyclopentyl-2-({4-[4-(dimethylamino)piperidin-1-yl]-2-ethoxyphenyl}amino)-7-methyl-7,9-dihydro-8H-purin-8-one, 9-cyclopentyl-2-({2-methoxy-4-[(1-methylpyrrolidin-3-yl)oxy]phenyl}amino)-7-methyl-7,9-dihydro-8H-purin-8-one, 9-cyclopentyl-2-({4-[2-(dimethylamino)ethoxy]-2-ethoxyphenyl}amino)-7-methyl-7,9-dihydro-8H-purin-8-one, 9-cyclopentyl-2-({2-ethoxy-4-[(1-methylpiperidin-4-yl)oxy]phenyl}amino)-7-methyl-7,9-dihydro-8H-purin-8-one, 9-cyclopentyl-2-({2-methoxy-4-[2-(4-methylpiperazin-1-yl)ethyl]phenyl}amino)-7-methyl-7,9-dihydro-8H-purin-8-one, 9-cyclopentyl-2-{[2-methoxy-4-(1-methylpiperidin-4-yl)phenyl]amino}-7-methyl-7,9-dihydro-8H-purin-8-one, 9-cyclopentyl-7-ethyl-2-{[2-methoxy-4-(4-methylpiperazin-1-yl)phenyl]amino}-7,9-dihydro-8H-purin-8-one, 9-cyclopentyl-7-ethyl-2-({2-methoxy-4-[(1-methylpiperidin-4-yl)oxy]phenyl}amino)-7,9-dihydro-8H-purin-8-one, 4-[(9-cyclopentyl-7-ethyl-8-oxo-8,9-dihydro-7H-purin-2-yl)amino]-3-methoxy-N-methylbenzamide, 9-cyclopentyl-7-ethyl-2-{[2-methoxy-4-(methylsulfonyl)phenyl]-amino}-7,9-dihydro-8H-purin-8-one, 4-[(9-cyclopentyl-7-ethyl-8-oxo-8,9-dihydro-7H-purin-2-yl)amino]-3-methoxy-N-(1-methylpiperidin-4-yl)benzamide, 4-[(9-cyclopentyl-7-ethyl-8-oxo-8,9-dihydro-7H-Purin-2-yl)amino]benzenesulfonamide, [9-cyclopentyl-2-({2-methoxy-4-[(1-methylpiperidin-4-yl)oxy]phenyl}amino)-8-oxo-8,9-dihydro-7H-purin-7-yl]acetonitrile, (9-cyclopentyl-2-{[2-methoxy-4-(4-methylpiperazin-1-yl)phenyl]amino}-8-oxo-8,9-dihydro-7H-purin-7-yl)acetonitrile, 4-{[7-(cyanomethyl)-9-cyclopentyl-8-oxo-8,9-dihydro-7H-purin-2-yl]amino}-3-methoxy-N-(1-methylpiperidin-4-yl)benzamide and 4-{[7-(cyanomethyl)-9-cyclopentyl-8-oxo-8,9-dihydro-7H-purin-2-yl]amino}benzenesulfonamide;

or a pharmaceutically acceptable salt thereof.

In a further embodiment there is provided a compound of formula (I) selected from 2-fluoro-4-[(9-isopropyl-7-methyl-8-oxo-8,9-dihydro-7H-purin-2-yl)-amino]-N-(1-methylpiperidin-4-yl)benzamide, 4-[(9-isopropyl-7-methyl-8-oxo-8,9-dihydro-7H-purin-2-yl)amino]-3-methoxy-N-(1-methylpiperidin-4-yl)benzamide, 4-[(9-isopropyl-7-methyl-8-oxo-8,9-dihydro-7H-purin-2-yl)amino]-3-methoxy-N-methylbenzamide, 9-isopropyl-2-({2-methoxy-4-[(1-methylpiperidin-4-yl)oxy]phenyl}-amino)-7-methyl-7,9-dihydro-8H-purin-8-one, 9-isopropyl-2-({2-methoxy-4-[(1-methylpyrrolidin-3-yl)oxy]-phenyl}amino)-7-methyl-7,9-dihydro-8H-purin-8-one, 9-isopropyl-2-{[2-methoxy-4-(4-methylpiperazin-1-yl)phenyl]-amino}-7-methyl-7,9-dihydro-8H-purin-8-one, 2-{[4-(4-ethylpiperazin-1-yl)-2-methoxyphenyl]amino}-9-isopropyl-7-methyl-7,9-dihydro-8H-purin-8-one, 2-{[2-ethoxy-4-(4-methylpiperazin-1-yl)phenyl]amino}-9-isopropyl-7-methyl-7,9-dihydro-8H-purin-8-one, 9-isopropyl-2-{[2-methoxy-4-(1-methylpiperidin-4-yl)phenyl]-amino}-7-methyl-7,9-dihydro-8H-purin-8-one, 9-isopropyl-2-{[2-methoxy-4-(4-methyl-1,4-diazepan-1-yl)phenyl]-amino}-7-methyl-7,9-dihydro-8H-purin-8-one, 4-[(9-cyclopentyl-7-methyl-8-oxo-8,9-dihydro-7H-purin-2-yl)-amino]-3-methoxybenzoic acid, 9-cyclopentyl-2-({2-methoxy-4-[(4-methylpiperazin-1-yl)carbonyl]-phenyl}amino)-7-methyl-7,9-dihydro-8H-purin-8-one, 9-cyclopentyl-2-[(2-methoxy-4-{[4-(1-methyl-piperidin-4-yl)-piperazin-1-yl]carbonyl}phenyl)amino]-7-methyl-7,9-dihydro-8H-purin-8-one, 4-[9-cyclopentyl-7-methyl-8-oxo-8,9-dihydro-7/1-purin-2-yl)-amino]-N-[3-(1H-imidazol-1-yl)propyl]-3-methoxybenzamide, 4-[(9-cyclopentyl-7-methyl-8-oxo-8,9-dihydro-7H-purin-2-yl)-amino]-N-[(1-isopropylpyrrolidin-3-yl)methyl]-3-methoxy-N-methylbenzamide, 4[(9-cyclopentyl-7-methyl-8-oxo-8,9-dihydro-7H-purin-2-yl)-amino]-N-[3-(dimethylamino)propyl]-3-methoxy-N-methylbenzamide, 9-cyclopentyl-2-[(4-{[(3R)-3-(dimethylamino)pyrrolidin-1-yl]-carbonyl}-2-methoxyphenyl)amino]-7-methyl-7,9-dihydro-8H-purin-8-one, 9-cyclopentyl-2-({2-methoxy-4-[(4-pyrrolidin-1-ylpiperidin-1-yl)-carbonyl]phenyl}amino)-7-methyl-7,9-dihydro-8H-purin-8-one, 9-cyclopentyl-2-[(2-methoxy-4-{[4-(2-methoxyethyl)piperazin-1-yl]-carbonyl}phenyl)amino]-7-methyl-7,9-dihydro-8H-purin-8-one, 4-[(9-cyclopentyl-7-methyl-8-oxo-8,9-dihydro-7H-purin-2-yl)-amino]-N-[4-(dimethylamino)cyclohexyl]-3-methoxybenzamide, 4-[(9-cyclopentyl-7-methyl-8-oxo-8,9-dihydro-7H-purin-2-yl)-amino]-3-methoxy-N-[1-(2-methoxyethyl)-piperidin-4-yl]benzamide, 4-[(9-cyclopentyl-7-methyl-8-oxo-8,9-dihydro-7H-purin-2-yl)-amino]-N-[(1-ethylpyrrolidin-2-yl)methyl]-3-methoxybenzamide, 4-[(9-cyclopentyl-7-methyl-8-oxo-8,9-dihydro-7H-purin-2-yl)-amino]-N-[4-(dimethylamino)butyl]-3-methoxybenzamide, 4-[(9-cyclopentyl-7-methyl-8-oxo-8,9-dihydro-7H-purin-2-yl)-amino]-N-[3-(dimethylamino)propyl]-3-methoxybenzamide, 4-[(9-cyclopentyl-7-methyl-8-oxo-8,9-dihydro-7H-purin-2-yl)-amino]-3-methoxy-N-(2-piperidin-1-ylethyl)benzamide, 4-[(9-cyclopentyl-7-methyl-8-oxo-8,9-dihydro-7H-purin-2-yl)-amino]-3-methoxy-N-[2-(4-methylpiperazin-1-yl)ethyl]benzamide, 4-[(9-cyclopentyl-7-methyl-8-oxo-8,9-dihydro-7H-purin-2-yl)-amino]-3-methoxy-N,N-dimethylbenzamide, 4-[(9-cyclopentyl-7-methyl-8-oxo-8,9-dihydro-7H-purin-2-yl)-amino]-3-methoxy-N-(4-pyrrolidin-1-ylbutyl)benzamide, 4-[(9-cyclopentyl-7-methyl-8-oxo-8,9-dihydro-7H-purin-2-yl)-amino]-N [2-(dimethyl-amino)ethyl]-3-methoxybenzamide, 4-[(9-cyclopentyl-7-methyl-8-oxo-8,9-dihydro-7H-purin-2-yl)-amino]-3-methoxy-N-[(3R)-pyrrolidin-3-yl]benzamide, 4-[(9-cyclopentyl-7-methyl-8-oxo-8,9-dihydro-7H-purin-2-yl)-amino]-3-methoxy-N-[(3S)-pyrrolidin-3-yl]benzamide, 2-{[2-methoxy-4-(4-methylpiperazin-1-yl)phenyl]amino}-7-methyl-9-piperidin-4-yl-7,9-dihydro-8H-purin-8-one, 2-[(4-benzoylphenyl)amino]-9-cyclopentyl-7-methyl-7,9-dihydro-8H-purin-8-one, 2-[(3-chloro-4-morpholin-4-ylphenyl)amino]-9-cyclopentyl-7-methyl-7,9-dihydro-8H-purin-8-one, 9-cyclopentyl-2-{[4-(2-hydroxyethyl)phenyl]amino}-7-methyl-7,9-dihydro-8H-purin-8-one, 9-cyclopentyl-2-[(4-isopropoxyphenyl)amino]-7-methyl-7,9-dihydro-8H-purin-8-one, 9-cyclopentyl-7-methyl-2-[(4-phenoxyphenyl)amino]-7,9-dihydro-8H-purin-8-one, 9-cyclopentyl-7-methyl-2-{[4-(1,3-oxazol-5-yl)phenyl]amino}-7,9-dihydro-8H-purin-8-one, 9-cyclopentyl-7-methyl-2-[(4-piperidin-1-ylphenyl)amino]-7,9-dihydro-8H-purin-8-one, 2-[(4-benzylphenyl)amino]-9-cyclopentyl-7-methyl-7,9-dihydro-8H-purin-8-one, 9-cyclopentyl-7-methyl-2-{[4-(1H-pyrazol-1-yl)phenyl]amino}-7,9-dihydro-8H-purin-8-one and 9-cyclopentyl-7-methyl-2-[(4-morpholin-4-ylphenyl)amino]-7,9-dihydro-8H-purin-8-one;

or a pharmaceutically acceptable salt thereof.

In a further embodiment there is provided a compound of formula (I) selected from:

4-[(9-cyclopentyl-7-methyl-8-oxo-8,9-dihydro-7H-purin-2-yl)amino]-3-methoxy-N-(1-methylpiperidin-4-yl)benzamide; 9-cyclopentyl-2-{[2-methoxy-4-(4-methylpiperazin-1-yl)phenyl]amino}-7-methyl-7,9-dihydro-8H-purin-8-one; 9-cyclopentyl-2-({2-methoxy-4-[(1-methylpiperidin-4-yl)oxy]phenyl}amino)-7-methyl-7,9-dihydro-8H-purin-8-one; 9-cyclopentyl-2-[(2-methoxy-4-morpholin-4-ylphenyl)amino]-7-methyl-7,9-dihydro-8H-purin-8-one; N-{4-[(9-cyclopentyl-7-methyl-8-oxo-8,9-dihydro-7H-purin-2-yl)amino]-3-methoxyphenyl}methanesulfonamide; 4-[(9-cyclopentyl-7-methyl-8-oxo-8,9-dihydro-7H-purin-2-yl)amino]-3-methoxy-N-methylbenzamide; 9-cyclopentyl-2-{[2-methoxy-4-(pyrrolidin-1-ylcarbonyl)phenyl]amino}-7-methyl-7,9-dihydro-8H-purin-8-one; 4-[(9-cyclopentyl-7-methyl-8-oxo-8,9-dihydro-7H-purin-2-yl)amino]-3-methoxy-N-(9-methyl-9-azabicyclo[3.3.1]non-3-yl)benzamide; 9-cyclopentyl-2-{[2-methoxy-4-(4-methyl-1,4-diazepan-1-yl)phenyl]amino}-7-methyl-7,9-dihydro-8H-purin-8-one; 9-cyclopentyl-2-{[2-ethoxy-4-(4-methyl-1,4-diazepan-1-yl)phenyl]amino}-7-methyl-7,9-dihydro-8H-purin-8-one; 9-cyclopentyl-2-({2-methoxy-4-[(1-methylpiperidin-4-yl)amino]phenyl}amino)-7-methyl-7,9-dihydro-8H-purin-8-one; 9-cyclopentyl-2-({4-[4-(dimethylamino)piperidin-1-yl]-2-ethoxyphenyl}amino)-7-methyl-7,9-dihydro-8H-purin-8-one; 9-cyclopentyl-2-({2-methoxy-4-[(1-methylpyrrolidin-3-yl)oxy]phenyl}amino)-7-methyl-7,9-dihydro-8H-purin-8-one; 9-cyclopentyl-2-({4-[2-(dimethylamino)ethoxy]-2-ethoxyphenyl}amino)-7-methyl-7,9-dihydro-8H-purin-8-one; 9-cyclopentyl-2-({2-ethoxy-4-[(1-methylpiperidin-4-yl)oxy]phenyl}amino)-7-methyl-7,9-dihydro-8H-purin-8-one; 9-cyclopentyl-2-({2-methoxy-4-[2-(4-methylpiperazin-1-yl)ethyl]phenyl}amino)-7-methyl-7,9-dihydro-8H-purin-8-one; 9-cyclopentyl-2-{[2-methoxy-4-(1-methylpiperidin-4-yl)phenyl]amino}-7-methyl-7,9-dihydro-8H-purin-8-one; 9-cyclopentyl-7-ethyl-2-{[2-methoxy-4-(4-methylpiperazin-1-yl)phenyl]amino}-7,9-dihydro-8H-purin-8-one; 9-cyclopentyl-7-ethyl-2-({2-methoxy-4-[(1-methylpiperidin-4-yl)oxy]phenyl}amino)-7,9-dihydro-8H-purin-8-one; 4-[(9-cyclopentyl-7-ethyl-8-oxo-8,9-dihydro-7H-purin-2-yl)amino]-3-methoxy-N-methylbenzamide; 9-cyclopentyl-7-ethyl-2-{[2-methoxy-4-(methylsulfonyl)phenyl]-amino}-7,9-dihydro-8H-purin-8-one; 4-[(9-cyclopentyl-7-ethyl-8-oxo-8,9-dihydro-7H-purin-2-yl)amino]-3-methoxy-N-(1-methylpiperidin-4-yl)benzamide; [9-cyclopentyl-2-({2-methoxy-4-[(1-methylpiperidin-4-yl)oxy]phenyl}amino)-8-oxo-8,9-dihydro-7H-purin-7-yl]acetonitrile; (9-cyclopentyl-2-{[2-methoxy-4-(4-methylpiperazin-1-yl)phenyl]amino}-8-oxo-8,9-dihydro-7H-purin-7-yl)acetonitrile; and 4-{[7-(cyanomethyl)-9-cyclopentyl-8-oxo-8,9-dihydro-7H-purin-2-yl]amino}-3-methoxy-N-(1-methylpiperidin-4-yl)benzamide;

or a pharmaceutically acceptable salt thereof.

In a further embodiment there is provided a compound of formula (I) selected from:

4-[(9-isopropyl-7-methyl-8-oxo-8,9-dihydro-7H-purin-2-yl)amino]-3-methoxy-N-(1-methylpiperidin-4-yl)benzamide; 4-[(9-isopropyl-7-methyl-8-oxo-8,9-dihydro-7H-purin-2-yl)amino]-3-methoxy-N-methylbenzamide; 9-isopropyl-2-({2-methoxy-4-[(1-methylpiperidin-4-yl)oxy]phenyl}-amino)-7-methyl-7,9-dihydro-8H-purin-8-one; 9-isopropyl-2-({2-methoxy-4-[(1-methylpyrrolidin-3-yl)oxy]-phenyl}amino)-7-methyl-7,9-dihydro-8H-purin-8-one; 9-isopropyl-2-{[2-methoxy-4-(4-methylpiperazin-1-yl)phenyl]-amino}-7-methyl-7,9-dihydro-8H-purin-8-one; 2-{[4-(4-ethylpiperazin-1-yl)-2-methoxyphenyl]amino}-9-isopropyl-7-methyl-7,9-dihydro-8H-purin-8-one; 2-{[2-ethoxy-4-(4-methylpiperazin-1-yl)phenyl]amino}-9-isopropyl-7-methyl-7,9-dihydro-8H-purin-8-one; 9-isopropyl-2-{[2-methoxy-4-(1-methylpiperidin-4-yl)phenyl]-amino}-7-methyl-7,9-dihydro-8H-purin-8-one; 9-isopropyl-2-{[2-methoxy-4-(4-methyl-1,4-diazepan-1-yl)phenyl]-amino}-7-methyl-7,9-dihydro-8H-purin-8-one; 4-[(9-cyclopentyl-7-methyl-8-oxo-8,9-dihydro-7H-purin-2-yl)-amino]-3-methoxybenzoic acid; 9-cyclopentyl-2-({2-methoxy-4-[(4-methylpiperazin-1-yl)carbonyl]-phenyl}amino)-7-methyl-7,9-dihydro-8H-purin-8-one; 9-cyclopentyl-2-[(2-methoxy-4-{[4-(1-methylpiperidin-4-yl)-piperazin-1-yl]carbonyl}phenyl)amino]-7-methyl-7,9-dihydro-8H-purin-8-one; 4-[(9-cyclopentyl-7-methyl-8-oxo-8,9-dihydro-7H-purin-2-yl)-amino]-N-[3-(1H-imidazol-1-yl)propyl]-3-methoxybenzamide; 4-[(9-cyclopentyl-7-methyl-8-oxo-8,9-dihydro-7H-purin-2-yl)-amino]-N-[(1-isopropylpyrrolidin-3-yl)methyl]-3-methoxy-N-methylbenzamide; 4-[(9-cyclopentyl-7-methyl-8-oxo-8,9-dihydro-7H-purin-2-yl)-amino]-N-[3-(dimethylamino)-propyl]-3-methoxy-N-methylbenzamide; 9-cyclopentyl-2-[(4-{[(3R)-3-(dimethylamino)-pyrrolidin-1-yl]-carbonyl}-2-methoxyphenyl)amino]-7-methyl-7,9-dihydro-8H-purin-8-one; 9-cyclopentyl-2-({2-methoxy-4-[(4-pyrrolidin-1-ylpiperidin-1-yl)-carbonyl]phenyl}-amino)-7-methyl-7,9-dihydro-8H-purin-8-one; 9-cyclopentyl-2-[(2-methoxy-4-{[4-(2-methoxyethyl)piperazin-1-yl]-carbonyl}phenyl)amino]-7-methyl-7,9-dihydro-8H-purin-8-one; 4-[(9-cyclopentyl-7-methyl-8-oxo-8,9-dihydro-7H-purin-2-yl)-amino]-N-[4-(dimethylamino)cyclohexyl]-3-methoxybenzamide; 4-[9-cyclopentyl-7-methyl-8-oxo-8,9-dihydro-7H-purin-2-yl)-amino]-3-methoxy-N-[1-(2-methoxyethyl)piperidin-4-yl]benzamide; 4-[(9-cyclopentyl-7-methyl-8-oxo-8,9-dihydro-7H-purin-2-yl)-amino]-N-[(1-ethylpyrrolidin-2-yl)methyl]-3-methoxybenzamide; 4-[(9-cyclopentyl-7-methyl-8-oxo-8,9-dihydro-7H-purin-2-yl)-amino]-N-[4-(dimethylamino)butyl]-3-methoxybenzamide; 4-[(9-cyclopentyl-7-methyl-8-oxo-8,9-dihydro-7H-purin-2-yl)-amino]-N-[3-(dimethyl-amino)propyl]-3-methoxybenzamide; 4-[(9-cyclopentyl-7-methyl-8-oxo-8,9-dihydro-7H-purin-2-yl)-amino]-3-methoxy-N-(2-piperidin-1-ylethyl)benzamide; 4-[(9-cyclopentyl-7-methyl-8-oxo-8,9-dihydro-7H-purin-2-yl)-amino]-3-methoxy-N-[2-(4-methylpiperazin-1-yl)ethyl]benzamide; 4-[(9-cyclopentyl-7-methyl-8-oxo-8,9-dihydro-7H-purin-2-yl)-amino]-3-methoxy-N,N-dimethylbenzamide; 4-[(9-cyclopentyl-7-methyl-8-oxo-8,9-dihydro-7H-purin-2-yl)-amino]-3-methoxy-N-(4-pyrrolidin-1-ylbutyl)benzamide; 4-[(9-cyclopentyl-7-methyl-8-oxo-8,9-dihydro-7H-purin-2-yl)-amino]-N-[2-(dimethylamino)-ethyl]-3-methoxybenzamide; 4-[(9-cyclopentyl-7-methyl-8-oxo-8,9-dihydro-7H-purin-2-yl)-amino]-3-methoxy-N-[(3R)-pyrrolidin-3-yl]benzamide; 4-[(9-cyclopentyl-7-methyl-8-oxo-8,9-dihydro-7H-purin-2-yl)-amino]-3-methoxy-N-[(3S)-pyrrolidin-3-yl]benzamide; and 2-{[2-methoxy-4-(4-methylpiperazin-1-yl)phenyl]amino}-7-methyl-9-piperidin-4-yl-7,9-dihydro-8H-purin-8-one;

or a pharmaceutically acceptable salt thereof.

Another aspect of the present invention provides a process for preparing a compound of formula (I) or a pharmaceutically acceptable salt thereof, which process comprises:

### Process a)

reacting a purinone of formula (II):

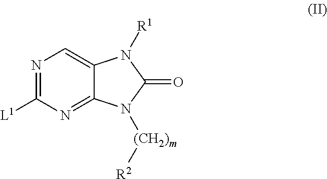

with an aniline of formula (III):

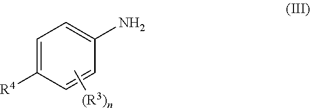

wherein L¹ is a displaceable group, and wherein the values of R¹, R², m, R³, R⁴ and n are as defined hereinbefore;

### Process b)

reacting a compound of formula (IV):

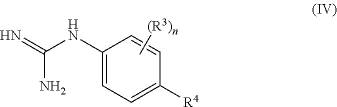

with a compound of formula (V):

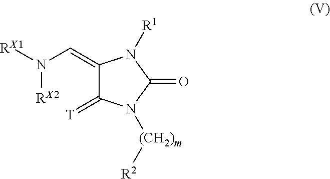

wherein T is O or S; R^{X1} and R^{X2} are each independently selected from C₁₋₆alkyl, C₃₋₆cycloalkyl or else R^{X1} and R^{X2} together with the nitrogen to which they are attached form a pyrrolidine or piperidine ring; and wherein the values of R¹, R², m, R³, R⁴ and n are as defined hereinbefore;

### Process c)

reacting a purinone of formula (VI):

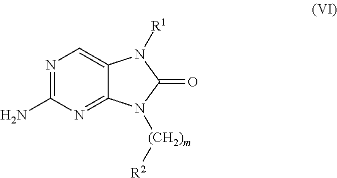

with a compound of formula (VII):

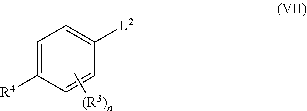

wherein L² is a displaceable group; and wherein the values of R¹, R², m, R³, R⁴ and n are as defined hereinbefore;

### Process d)

reacting a purinone of formula (VIII):

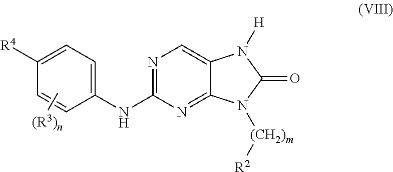

with a compound of formula (IX):

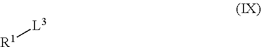

wherein L³ is a displaceable group; and wherein the values of R¹, R², m, R³, R⁴ and n are as defined hereinbefore;

### Process e)

reacting a purinone of formula (X):

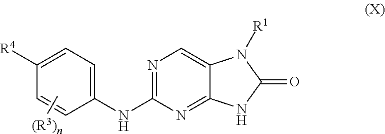

with a compound of formula (XI):

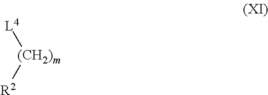

wherein L⁴ is a displaceable group; and wherein the values of R¹, R², m, R³, R⁴ and n are as defined hereinbefore; or

### Process f)

reacting a pyrimidine of formula (XII):

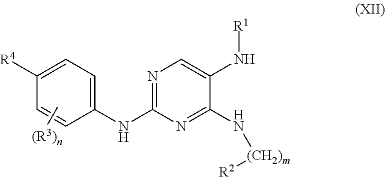

with a compound of formula (XIII):

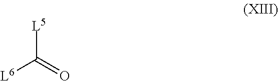

wherein L⁵ and L⁶ are displaceable groups; and wherein the values of R¹, R², m, R³, R⁴ and n are as defined hereinbefore;
   and optionally removing any protecting groups to provide a compound of formula (I) and optionally, thereafter, carrying out one or both of the following steps:
   i) converting a compound of formula (I) into another compound of formula (I);
   ii) forming a pharmaceutically acceptable salt.
   Further information relating to the above processes is provided below.

### Process a)

Suitable values for L¹ are for example, a halo, for example a chloro, bromo or iodo, or an optionally fluorinated alkylsulfonyloxy, for example a methanesulfonyloxy or trifluoromethanesulfonyloxy group; or an optionally substituted arylsulfonyloxy group, wherein said optionally substitution is on the aryl ring, wherein said optional substituents include one or more units selected from C₁₋₃alkyl, halo and nitro, giving for example a phenyl-4-sulfonyloxy or toluene-4-sulfonyloxy group.

Purinones of formula (II) and anilines of formula (III) may be reacted together in the absence of solvent or using a polar solvent, for example an aprotic solvent such as N-methylpyrrolidinone, or for example a protic solvent such as isopropanol, using microwave or conventional heating, to a temperature in the range 140-190° C., optionally in the presence of a suitable acid, for example a sulfonic acid such as p-toluenesulfonic acid, or for example a mineral acid such as hydrochloric acid. Purinones of formula (II) wherein L¹ is chloro may be prepared according to Scheme 1.

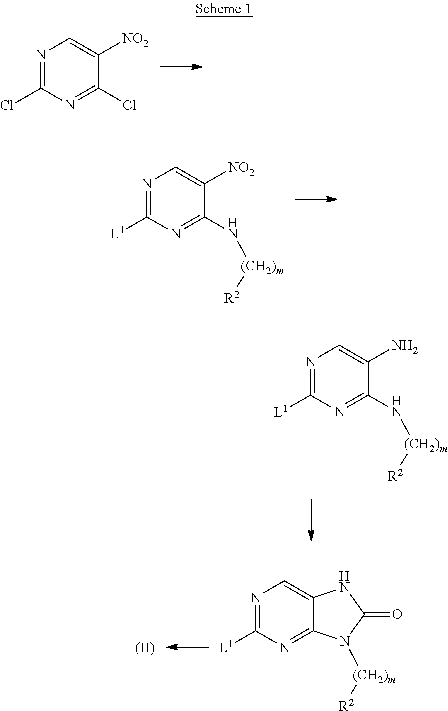

Anilines of formula (III) are commercially available compounds, or they are known in the literature, or they are prepared by standard processes known in the art.

### Process b)

Compounds of formula (IV) and compounds of formula (V) can be reacted together in a suitable solvent, for example a polar aprotic solvent such as N-methylpyrrolidinone, or for example a polar protic solvent such as butanol, using conventional or microwave heating, at a temperature around 150-170° C. in the presence of a suitable base such as an alkali metal hydride base, for example, sodium hydride, or for example an alkoxide base such as sodium methoxide, or for example an inorganic carbonate base, such as potassium carbonate.

Compounds of formula (V) where T is O and R^{X1} and R^{X2} are methyl may be prepared according to Scheme 2:

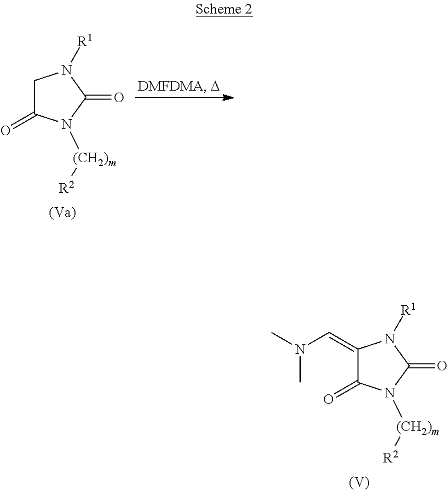

Compounds of formula (IV) and (Va) are commercially available compounds, or they are known in the literature, or they are prepared by standard processes known in the art.

### Process c)

Suitable values for L² are halo, for example bromo or iodo, or a sulphonyloxy group, for example a C₁₋₆alkylsulfonyloxy group optionally substituted by fluoro, such as a trifluoromethanesulfonyloxy group.

Compounds of formula (VI) and amines of formula (VII) may be reacted together under standard Buchwald conditions (for example see J. Am. Chem. Soc., 118, 7215; J. Am. Chem. Soc., 119, 8451; J. Org. Chem., 62, 1568 and 6066) for example in the presence of a palladium source, such as palladium acetate, in a suitable solvent for example an aprotic aromatic solvent such as toluene, benzene or xylene, with a suitable base for example an alkali metal carbonate base such as caesium carbonate or an alkoxide base such as potassium-t-butoxide, in the presence of a suitable ligand such as 2,2′-bis(diphenylphosphino)-1,1′-binaphthyl and at a temperature in the range of 25-80° C.

The synthesis of compounds of formula (VI) is described in Scheme 1.

Compounds of formula (VII) are commercially available compounds, or they are known in the literature, or they are prepared by standard processes known in the art.

### Processes d and e)

Suitable values of L³ and L⁴ are for example, a halo, for example a chloro, bromo or iodo, or an optionally fluorinated alkylsulfonyloxy, for example a methanesulfonyloxy or trifluoromethanesulfonyloxy group; or an optionally substituted arylsulfonyloxy group, wherein said optionally substitution is on the aryl ring, wherein said optional substituents include one or more units selected from C₁₋₃alkyl, halo and nitro, giving for example a phenyl-4-sulfonyloxy or toluene-4-sulfonyloxy group.

Compound of formula (VIII) and compound of formula (IX) can be reached together under standard alkylation conditions that are well known in the art which will generally involve the use of a base, for example an tertiary amine base such as triethylamine, or for example an aromatic base, such as pyridine, or an inorganic base such as a metal carbonate or an alkali metal hydride. A base may not be necessary if there is a sufficiently basic group elsewhere on the compound of formula (VIII).

### Process f)

Suitable values of L⁵ and L⁶ include halo, for example chloro or bromo, or an optionally substituted hydrocarbyloxy group, for example an optionally substituted C₁₋₆alkoxy group, or an optionally substituted aryloxy group, such as a phenoxy group, or for example a bulky alkanoyloxy group, for example a t-butylalkanoyloxy group or other known leaving group such as an imidazoyl group. It is not possible to be exhaustive about the possible values that L⁵ and L⁶ could reasonably take, and the skilled person is well aware of what values will be suitable for this type of reaction.

Compounds of formula (XII) and compounds of formula (XIII) may be reacted together in the presence of a suitable solvent for example an ethereal solvent such as tetrahydrofuran, in the presence of a base, for example a tertiary amine base such as triethylamine, or for example an aromatic base such as pyridine, optionally in the presence of a nucleophilic catalyst for example 4-(N,N-dimethylamino)pyridine. Reaction conditions for this type of transformation are well known in the art.

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

As stated hereinbefore the compounds defined in the present invention possess anti-cancer activity which is believed to arise from TTK inhibitory activity of the compounds. These properties may be assessed, for example, using the procedures set out below:—

### Biological Assays

The following assays can be used to measure the effects of the compounds of the present invention as inhibitors of the kinase TTK and as inhibitors in vitro of the spindle checkpoint.

### (a) In Vitro TTK Kinase Assay 1

The assay uses AlphaScreen technology (Gray et al., Analytical Biochemistry, 2003, 313: 234-245) to determine the ability of test compounds to inhibit phosphorylation by recombinant TTK.

N-terminal GST tagged full length human TTK kinase (GenBank Accession No. NM_{—}003318) was expressed in insect cells and purified via the GST epitope tag, using standard affinity purification techniques.

Test compounds were prepared as 10 mM stock solutions in dimethyl sulphoxide (DMSO) and diluted into water as required to give a range of final assay concentrations. Aliquots (2 μL) of each compound dilution were placed into wells of a Greiner 384-well low volume white polystyrene plate (Greiner Bio-one). A 10 μL mixture of recombinant purified TTK enzyme, biotinylated peptide substrate (Biotin-Ahx-GLARHTDDEMTGYVATRWYR-NH₂), 10 μM adenosine triphosphate (ATP) and a buffer solution [comprising 25 mM HEPES pH 7.4, 0.01% v/v Tween™-20, 1 mM Dithiothreitol (DTT) and 10 mM MgCl₂] was incubated at room temperature for 60 minutes.

Control wells that produced a maximum signal corresponding to maximum enzyme activity were created by adding 5% DMSO instead of test compound. Control wells that produced a minimum signal corresponding to fully inhibited enzyme were created by adding EDTA to a concentration of 83 mM instead of test compound.

Each reaction was stopped by the addition of EDTA to a concentration of 83 mM and phosphorylated substrate was captured and detected in a buffer comprising 0.3% bovine serum albumin (BSA), 200 mM NaCl and 25 mM HEPES pH 7.4 containing 40 ng/μL AlphaScreen Streptavidin donor and Protein A acceptor beads (Perkin Elmer) and phosphospecific antibody diluted 1:2000 (CST Catalogue No 9211). The resultant signals arising from laser light excitation at 680 nm were read using a Packard Envision instrument. The mean data values for each test compound concentration, EDTA treated control wells and 100% inhibition control wells were used to determine the test compounds IC₅₀ value. IC₅₀ value is the concentration of test compound that inhibits 50% of kinase activity.

### (b) In Vitro TTK Kinase Assay 2

Inhibitors of the kinase activity of TTK were identified using the Caliper LabChip LC3000 (Caliper Life Sciences), which utilises microfluidic, chips to measure the conversion of a fluorescent-labelled peptide to a phosphorylated product (Pommereau et al (2004) J. Biomol Screen (5) 409-416) by recombinant TTK.

N-terminal GST tagged full length human TTK kinase (GenBank Accession No. NM_{—}003318) was expressed in insect cells and purified via the GST epitope tag, using standard affinity purification techniques.

Test compounds were prepared as 10 mM stock solutions in dimethyl sulfoxide (DMSO) and further diluted in DMSO to give a range of final assay concentrations. Aliquots (120 nL) of each compound dilution were placed into wells of a Greiner 384-well low volume white polystyrene plate (Greiner Catalogue Number: 784075) using an Echo acoustic liquid handler (Labcyte Inc). A 12 μL mixture of recombinant purified TTK enzyme, fluorescein isothiocyanate (FITC)-labelled peptide substrate (FITC-DHTGFLTEYVATR-CONH₂), 12 μM adenosine triphosphate (ATP) and a buffer solution [comprising 50 mM HEPES pH 7.5, 0.015% v/v Brij™-35, 1 mM Dithiothreitol (DTT) and 10 mM MgCl₂] was incubated at room temperature for 25 minutes.

Control wells that produced a maximum signal corresponding to maximum enzyme activity were created by adding DMSO to a final concentration of 1% instead of test compound. Control wells producing a minimum signal corresponding to fully inhibited enzyme were created by adding staurosporine to a concentration of 100 μM instead of test compound.

Each reaction was stopped by the addition of EDTA to a concentration of 40 mM in a solution which also comprised 0.1% coating reagent (Caliper LS), 100 mM HEPES pH 7.5, 0.015% v/v Brij™-35 and 5% DMSO. The stopped enzyme reactions were sipped through capillaries onto a Caliper chip where the peptide substrate and phosphorylated product were separated and detected via laser-induced fluorescence. The mean data values for each test compound concentration, DMSO control wells and 100% inhibition control wells were used to determine the IC₅₀ value of the test compound.

### (c) Spindle Checkpoint Abrogation Assay

Chromosome condensation in mitosis is accompanied by phosphorylation of histone H3 on serine 10. Dephosphorylation begins in anaphase and ends at early telophase, thus histone H3 serine 10 phosphorylation acts as an excellent mitotic marker. Paclitaxol is a microtubule stabilising drug which perturbs microtubule dynamics, invokes the spindle checkpoint and arrests cells in mitosis. These cells are positive for histone H3 serine 10 phosphorylation. Inhibition of the spindle checkpoint overrides the mitotic block in the presence of paclitaxol and the histone H3 serine 10 endpoint is used as a marker to determine the ability of compounds of the present invention to exit mitotic arrest prematurely.

Cells of the human colon tumour cell line HT29 were seeded into 96 well black plates (Costar, Catalogue No 3904) in phenol red free Dulbecco's Modified Eagles Medium (DMEM) supplemented with 10% (v/v) FCS and 1% (v/v) L-Glutamine and incubated overnight at 37° C. Paclitaxol was added to the cells at a concentration of 7.8 nM and the cells incubated overnight prior to compound dosing. Test compounds were solubilised in DMSO, diluted to give a range of final assay concentrations, added to cells and incubated for 5 h at 37° C. After 5 hours, cells were fixed in 3.7% (v/v) formaldehyde then permeabilised and blocked for 10 minutes in 100 μL 0.5% (v/v) Triton™ X-100, 1% (w/v) bovine serum albumin (BSA) in phosphate buffered saline (PBS). After washing with PBS, 50 μL primary antibody (1:500 dilution of rabbit anti-phosphohistone H3 (Upstate Catalogue No 06-570) in 1% BSA, 0.05% Tween™ 20) was added to the cells that were left for 1 hour at room temperature. Cells were again washed with PBS and incubated with 50 μL secondary antibody (1:1000 Alexa Fluor 488 goat anti-rabbit (Molecular Probes Cat No A-11008) and Hoechst 33342 (Molecular Probes Cat. No. H-3570) diluted in PBS 0.05% (v/v) Tween™ 20 (1:10000 dilution) and left for 1 hour at room temperature in the dark. Cells were washed with PBS then covered with fresh PBS and stored at 4° C. until analysis. Images are acquired and analysed in an automated manner using the Cellomics ArrayScan II or VTi. In this assay both hoechst 33342 and phosphohistone H3 staining are measured. Hoechst 33342 labels DNA and is used to generate a valid cell count while phosphohistone H3 staining determines the number of mitotic cells. Inhibition of TTK leads to a decrease in the population of histone H3 Serine 10 positive cells, indicating inappropriate exit from mitosis in the presence of the spindle toxin. The raw assay data were analysed by non-linear regression analysis and used to determine an IC₅₀ value for each compound.

IC₅₀ values for compounds of the invention when tested in one or more of the above assays are typically less than 100 μM.

The compounds of formula (I) have activity as pharmaceuticals, in particular as modulators or inhibitors of TTK activity, and may be used in the treatment of proliferative and hyperproliferative diseases/conditions, including solid tumours such as carcinomas and sarcomas and the leukaemias and lymphoid malignancies. Examples of these proliferative and hyperproliferative diseases/conditions include the following cancers:
(1) carcinoma, including that of the bladder, brain, breast, colon, kidney, liver, lung, ovary, pancreas, prostate, stomach, cervix, colon, thyroid and skin;
(2) hematopoietic tumours of lymphoid lineage, including acute lymphocytic leukaemia, B-cell lymphoma and Burketts lymphoma;
(3) hematopoietic tumours of myeloid lineage, including acute and chronic myelogenous leukaemias and promyelocytic leukaemia;
(4) tumours of mesenchymal origin, including fibrosarcoma and rhabdomyosarcoma; and
(5) other tumours, including melanoma, seminoma, tetratocarcinoma, neuroblastoma and glioma.

In one embodiment the compounds of the invention are useful in the treatment of tumours of the bladder, breast and prostate and multiple myeloma.

Thus, the present invention provides a compound of formula (I), or a pharmaceutically-acceptable salt thereof, as herein defined for use in therapy.

According to a further aspect of the present invention there is provided a compound of the formula (I), or a pharmaceutically acceptable salt thereof, as defined hereinbefore for use in a method of treatment of the human or animal body by therapy.

In a further aspect, the present invention provides the use of a compound of formula (I), or a pharmaceutically acceptable salt, as herein defined in the manufacture of a medicament for use in therapy.

In the context of the present specification, the term “therapy” also includes “prophylaxis” unless there are specific indications to the contrary. The terms “therapeutic” and “therapeutically” should be construed accordingly.

The invention also provides a method of treating cancer which comprises administering to a patient in need thereof a therapeutically effective amount of a compound of formula (I), or a pharmaceutically acceptable salt thereof, as herein defined.

We have found that the compounds defined in the present invention, or a pharmaceutically acceptable salt thereof, are effective anti-cancer agents which property is believed to arise from modulating or inhibiting TTK activity. Accordingly the compounds of the present invention are expected to be useful in the treatment of diseases or medical conditions mediated alone or in part by TTK, i.e. the compounds may be used to produce an TTK inhibitory effect in a warm-blooded animal in need of such treatment.

Thus the compounds of the present invention provide a method for treating cancer characterised by inhibition of TTK, i.e. the compounds may be used to produce an anti-cancer effect mediated alone or in part by the inhibition of TTK.

It is in addition expected that a compound of the present invention will possess activity against a range of leukaemias, lymphoid malignancies and solid tumours such as carcinomas and sarcomas in tissues such as the liver, kidney, bladder, prostate, breast and pancreas. In one embodiment compounds of the invention are expected to slow advantageously the growth of primary and recurrent solid tumours of, for example, the skin, colon, thyroid, lungs and ovaries.

Thus according to this aspect of the invention there is provided a compound of the formula (I), or a pharmaceutically acceptable salt thereof, as defined herein for use as a medicament.

According to a further aspect of the invention there is provided the use of a compound of the formula (I), or a pharmaceutically acceptable salt thereof, as defined herein in the manufacture of a medicament for the production of a TTK inhibitory effect in a warm-blooded animal such as man.

According to this aspect of the invention there is provided a compound of formula (I), or a pharmaceutically acceptable salt thereof, as defined herein for use in the production of a TTK inhibitory effect in a warm-blooded animal such as man.

According to this aspect of the invention there is provided the use of a compound of the formula (I), or a pharmaceutically acceptable salt thereof, as defined herein in the manufacture of a medicament for the production of an anti-cancer effect in a warm-blooded animal such as man.

According to this aspect of the invention there is provided a compound of the formula (I), or a pharmaceutically acceptable salt thereof, as defined herein for use in the production of an anti-cancer effect in a warm-blooded animal such as man.

According to a further feature of the invention, there is provided the use of a compound of the formula (I), or a pharmaceutically acceptable salt thereof, as defined herein in the manufacture of a medicament for the treatment of melanoma, papillary thyroid tumours, cholangiocarcinomas, colon cancer, ovarian cancer, lung cancer, leukaemias, lymphoid malignancies, multiple myeloma, carcinomas and sarcomas in the liver, kidney, bladder, prostate, breast and pancreas, and primary and recurrent solid tumours of the skin, colon, thyroid, lungs and ovaries.

According to this feature of the invention, there is provided a compound of the formula (I), or a pharmaceutically acceptable salt thereof, as defined herein for use in the treatment of melanoma, papillary thyroid tumours, cholangiocarcinomas, colon cancer, ovarian cancer, lung cancer, leukaemias, lymphoid malignancies, multiple myeloma, carcinomas and sarcomas in the liver, kidney, bladder, prostate, breast and pancreas, and primary and recurrent solid tumours of the skin, colon, thyroid, lungs and ovaries.

According to a further aspect of the invention there is provided the use of a compound of the formula (I), or a pharmaceutically acceptable salt thereof, as defined herein in the production of a TTK inhibitory effect in a warm-blooded animal such as man.

According to this aspect of the invention there is provided the use of a compound of the formula (I), or a pharmaceutically acceptable salt thereof, as defined herein in the production of an anti-cancer effect in a warm-blooded animal such as man.

According to a further feature of the invention, there is provided the use of a compound of the formula (I), or a pharmaceutically acceptable salt thereof, as defined herein in the treatment of melanoma, papillary thyroid tumours, cholangiocarcinomas, colon cancer, ovarian cancer, lung cancer, leukaemias, lymphoid malignancies, multiple myeloma, carcinomas and sarcomas in the liver, kidney, bladder, prostate, breast and pancreas, and primary and recurrent solid tumours of the skin, colon, thyroid, lungs and ovaries.

According to a further feature of this aspect of the invention there is provided a method for producing a TTK inhibitory effect in a warm-blooded animal, such as man, in need of such treatment which comprises administering to said animal an effective amount of a compound of formula (I), or a pharmaceutically acceptable salt thereof, as defined herein.

According to a further feature of this aspect of the invention there is provided a method for producing an anti-cancer effect in a warm-blooded animal, such as man, in need of such treatment which comprises administering to said animal an effective amount of a compound of formula (I), or a pharmaceutically acceptable salt thereof, as defined herein.

According to an additional feature of this aspect of the invention there is provided a method of treating melanoma, papillary thyroid tumours, cholangiocarcinomas, colon cancer, ovarian cancer, lung cancer, leukaemias, lymphoid malignancies, multiple myeloma, carcinomas and sarcomas in the liver, kidney, bladder, prostate, breast and pancreas, and primary and recurrent solid tumours of the skin, colon, thyroid, lungs and ovaries, in a warm-blooded animal, such as man, in need of such treatment which comprises administering to said animal an effective amount of a compound of formula (I) or a pharmaceutically acceptable salt thereof as defined herein.

In a further aspect of the invention there is provided a pharmaceutical composition which comprises a compound of the formula (I), or a pharmaceutically acceptable salt thereof, as defined herein in association with a pharmaceutically-acceptable diluent or carrier for use in the production of a TTK inhibitory effect in a warm-blooded animal such as man.

In a further aspect of the invention there is provided a pharmaceutical composition which comprises a compound of the formula (I), or a pharmaceutically acceptable salt thereof, as defined herein in association with a pharmaceutically-acceptable diluent or carrier for use in the production of an anti-cancer effect in a warm-blooded animal such as man.

In a further aspect of the invention there is provided a pharmaceutical composition which comprises a compound of the formula (I), or a pharmaceutically acceptable salt thereof, as defined herein in association with a pharmaceutically-acceptable diluent or carrier for use in the treatment of melanoma, papillary thyroid tumours, cholangiocarcinomas, colon cancer, ovarian cancer, lung cancer, leukaemias, lymphoid malignancies, multiple myeloma, carcinomas and sarcomas in the liver, kidney, bladder, prostate, breast and pancreas, and primary and recurrent solid tumours of the skin, colon, thyroid, lungs and ovaries in a warm-blooded animal such as man.

The compounds of formula (I) and pharmaceutically acceptable salts thereof may be used on their own but will generally be administered in the form of a pharmaceutical composition in which the formula (I) compound or salt (active ingredient) is in association with a pharmaceutically acceptable adjuvant, diluent or carrier. Depending on the mode of administration, the pharmaceutical composition may comprise from 0.01 to 99% w (percent by weight), from 0.05 to 80% w, from 0.10 to 70% w, and or even from 0.10 to 50% w, of active ingredient, all percentages by weight being based on total composition.

The present invention also provides a pharmaceutical composition comprising a compound of formula (I), or a pharmaceutically acceptable salt thereof, as herein defined, in association with a pharmaceutically acceptable adjuvant, diluent or carrier.

The invention further provides a process for the preparation of a pharmaceutical composition of the invention which comprises mixing a compound of formula (I), or a pharmaceutically acceptable salt thereof, as herein defined, with a pharmaceutically acceptable adjuvant, diluent or carrier.

The pharmaceutical compositions may be administered topically (e.g. to the skin or to the lung and/or airways) in the form, e.g., of creams, solutions, suspensions, heptafluoroalkane aerosols and dry powder formulations; or systemically, e.g. by oral administration in the form of tablets, capsules, syrups, powders or granules; or by parenteral administration in the form of solutions or suspensions; or by subcutaneous administration; or by rectal administration in the form of suppositories; or transdermally.

The compositions of the invention may be obtained by conventional procedures using conventional pharmaceutical excipients, well known in the art. Thus, compositions intended for oral use may contain, for example, one or more colouring, sweetening, flavouring and/or preservative agents.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Suppository formulations may be prepared by mixing the active ingredient with a suitable non-irritating excipient which is solid at ordinary temperatures but liquid at the rectal temperature and will therefore melt in the rectum to release the drug. Suitable excipients include, for example, cocoa butter and polyethylene glycols.

Topical formulations, such as creams, ointments, gels and aqueous or oily solutions or suspensions, may generally be obtained by formulating an active ingredient with a conventional, topically acceptable, vehicle or diluent using conventional procedure well known in the art.

Compositions for administration by insufflation may be in the form of a finely divided powder containing particles of average diameter of, for example, 30 μm or much less, the powder itself comprising either active ingredient alone or diluted with one or more physiologically acceptable carriers such as lactose. The powder for insufflation is then conveniently retained in a capsule containing, for example, 1 to 50 mg of active ingredient for use with a turbo-inhaler device, such as is used for insufflation of the known agent sodium cromoglycate.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

The size of the dose for therapeutic purposes of a compound of the invention will naturally vary according to the nature and severity of the conditions, the age and sex of the animal or patient and the route of administration, according to well known principles of medicine.

In general, a compound of the invention will be administered so that a daily dose in the range, for example, from 0.1 mg to 1000 mg active ingredient per kg body weight is received, given if required in divided doses. However the daily dose will necessarily be varied depending upon the host treated, the particular route of administration, and the severity of the illness being treated. Accordingly the optimum dosage may be determined by the practitioner who is treating any particular patient. In general lower doses will be administered when a parenteral route is employed. Thus, for example, for intravenous administration, a dose in the range, for example, from 0.1 mg to 30 mg active ingredient per kg body weight will generally be used. Similarly, for administration by inhalation, a dose in the range, for example, from 0.1 mg to 25 mg active ingredient per kg body weight will generally be used. Oral administration is however preferred. For example, a formulation intended for oral administration to humans will generally contain, for example, from 0.1 mg to 2 g of active ingredient.

For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

The anti cancer treatment defined hereinbefore may be applied as a sole therapy or may involve, in addition to the compound of the invention, conventional surgery or radiotherapy or chemotherapy. Such chemotherapy may include one or more of the following categories of anti-tumour agents:—

(i) other antiproliferative/antineoplastic drugs and combinations thereof, as used in medical oncology, such as alkylating agents (for example cis platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5 fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);

(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5*-reductase such as finasteride;

(iii) anti-invasion agents (for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341) and N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661), and metalloproteinase inhibitors like marimastat, inhibitors of urokinase plasminogen activator receptor function or antibodies to Heparanase);

(iv) inhibitors of growth factor function: for example such inhibitors include growth factor antibodies and growth factor receptor antibodies (for example the anti erbB2 antibody trastuzumab [Herceptin™], the anti-EGFR antibody panitumumab, the anti erbB 1 antibody cetuximab [Erbitux, C225] and any growth factor or growth factor receptor antibodies disclosed by Stern et al. Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI 774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib, inhibitors of the hepatocyte growth factor family, inhibitors of the platelet-derived growth factor family such as imatinib, inhibitors of serine/threonine kinases (for example Ras/Raf signalling inhibitors such as farnesyl transferase inhibitors, for example sorafenib (BAY 43-9006)), inhibitors of cell signalling through MEK and/or AKT kinases, inhibitors of the hepatocyte growth factor family, c-kit inhibitors, abl kinase inhibitors, IGF receptor (insulin-like growth factor) kinase inhibitors; aurora kinase inhibitors (for example AZD1152, PH739358, VX-680, MLN8054, R763, MP235, MP529, VX-528 AND AX39459) and cyclin dependent kinase inhibitors such as CDK2 and/or CDK4 inhibitors;

(v) antiangiogenic agents such as those which inhibit the effects of vascular endothelial growth factor, [for example the anti vascular endothelial cell growth factor antibody bevacizumab (Avastin™) and VEGF receptor tyrosine kinase inhibitors such as 4-(4-bromo-2-fluoroanilino)-6-methoxy-7-(1-methylpiperidin-4-ylmethoxy)quinazoline (ZD6474; Example 2 within WO 01/32651), 4-(4-fluoro-2-methylindol-5-yloxy)-6-methoxy-7-(3-pyrrolidin-1-ylpropoxy)quinazoline (AZD2171; Example 240 within WO 00/47212), vatalanib (PTK787; WO 98/35985) and SU11248 (sunitinib; WO 01/60814), compounds such as those disclosed in International Patent Applications WO97/22596, WO 97/30035, WO 97/32856 and WO 98/13354 and compounds that work by other mechanisms (for example linomide, inhibitors of integrin avb3 function and angiostatin)];

(vi) vascular damaging agents such as Combretastatin A4 and compounds disclosed in International Patent Applications WO 99/02166, WO 00/40529, WO 00/41669, WO 01/92224, WO 02/04434 and WO 02/08213;

(vii) antisense therapies, for example those which are directed to the targets listed above, such as ISIS 2503, an anti-ras antisense;

(viii) gene therapy approaches, including for example approaches to replace aberrant genes such as aberrant p53 or aberrant BRCA1 or BRCA2, GDEPT (gene directed enzyme pro drug therapy) approaches such as those using cytosine deaminase, thymidine kinase or a bacterial nitroreductase enzyme and approaches to increase patient tolerance to chemotherapy or radiotherapy such as multi drug resistance gene therapy; and

(ix) immunotherapy approaches, including for example ex vivo and in vivo approaches to increase the immunogenicity of patient tumour cells, such as transfection with cytokines such as interleukin 2, interleukin 4 or granulocyte macrophage colony stimulating factor, approaches to decrease T cell energy, approaches using transfected immune cells such as cytokine transfected dendritic cells, approaches using cytokine transfected tumour cell lines and approaches using anti idiotypic antibodies.

According to this aspect of the invention there is provided a pharmaceutical product comprising a compound of formula (I) as defined hereinbefore with an additional anti-tumour substance as defined hereinbefore for the conjoint treatment of cancer.

In the above other pharmaceutical composition, process, method, use and medicament manufacture features, the alternative embodiments of the compounds of the invention described herein also apply.

### EXAMPLES

The invention will now be illustrated in the following Examples in which, generally:

(i) operations were carried out at ambient temperature, i.e. in the range 17 to 25° C. and under an atmosphere of an inert gas such as nitrogen or argon unless otherwise stated;

(ii) in general, the course of reactions was followed by thin layer chromatography (TLC) and/or analytical high pressure liquid chromatography (HPLC); the reaction times that are given are not necessarily the minimum attainable;

(iii) when necessary, organic solutions were dried over anhydrous MgSO₄, work-up procedures were carried out using traditional layer separating techniques, evaporations were carried out either by rotary evaporation in vacuo or in a Genevac HT-4/EZ-2.

(iv) yields, where present, are not necessarily the maximum attainable, and when necessary, reactions were repeated if a larger amount of the reaction product was required;

(v) in general, the structures of the end-products of the Formula (I) were confirmed by nuclear magnetic resonance (NMR) and/or mass spectral techniques; electrospray mass spectral data were obtained using a Waters ZMD or Waters ZQ LC/mass spectrometer acquiring both positive and negative ion data, generally, only ions relating to the parent structure are reported; proton NMR chemical shift values were measured on the delta scale using either a Bruker Avance DPX300 spectrometer operating at a field strength of 300 MHz, or a Bruker Avance DPX400 operating at 400 MHz. Unless otherwise stated, NMR spectra were obtained at 400 MHz in d⁶-dimethylsulfoxide. The following abbreviations have been used: s, singlet; d, doublet; t, triplet; q, quartet; m, multiplet; br, broad;

(vi) unless stated otherwise compounds containing an asymmetric carbon and/or sulphur atom were not resolved;

(vii) intermediates were not necessarily fully purified but their structures and purity were assessed by TLC, analytical HPLC, infra-red (IR) and/or NMR analysis;

(viii) unless otherwise stated, column chromatography (by the flash procedure, FCC) and medium pressure liquid chromatography (MPLC) were performed on Merck Kieselgel silica (Art. 9385) or on Silicycle cartridges (40-63 μm silica, 12 to 120 g weight) using an Isco Combi Flash Companion system.

(ix) Preparative HPLC was performed on C18 reversed-phase silica, for example on a Waters ‘Xterra’ or ‘Bridge’ preparative reversed-phase column (5 μm silica, 19 mm diameter, 100 mm length) or on a Phenomenex “Gemini” or ‘AXIA’ preparative reversed-phase column (5 μm silica, 110 A, 21.1 mm diameter, 100 mm length) using decreasingly polar mixtures as eluent, for example (containing 1% formic acid or 1% aqueous NH₄OH (d=0.88)) as solvent A and acetonitrile as solvent B; either of the following preparative HPLC methods were used:

Method A: a solvent gradient over 9.5 minutes, at 25 mL per minute, from a 85:15 mixture of solvents A and B respectively to a 5:95 mixture of solvents A and B.

Method B: a solvent gradient over 9.5 minutes, at 25 mL per minute, from a 60:40 mixture of solvents A and B respectively to a 5:95 mixture of solvents A and B.

(x) the following analytical HPLC methods were used; in general, reversed-phase silica was used with a flow rate of about 1 mL per minute and detection was by Electrospray Mass Spectrometry and by UV absorbance at a wavelength of 254 nm; for each method Solvent A was water and Solvent B was acetonitrile; the following columns and solvent mixtures were used:—

Analytical HPLC was performed on C18 reversed-phase silica, on a Phenomenex “Gemini” preparative reversed-phase column (5 μm silica, 110 A, 2 mm diameter, 50 mm length) using decreasingly polar mixtures as eluent, for example decreasingly polar mixtures of water (containing 0.1% formic acid or 0.1% ammonia) as solvent A and acetonitrile as solvent B; the following analytical HPLC method was used:

A solvent gradient over 4 minutes, at approximately 1 mL per minute, from a 95:5 mixture of solvents A and B respectively to a 5:95 mixture of solvents A and B.

(xi) where certain compounds were obtained as an acid-addition salt, for example a mono-hydrochloride salt or a di-hydrochloride salt, the stoichiometry of the salt was based on the number and nature of the basic groups in the compound, the exact stoichiometry of the salt was generally not determined, for example by means of elemental analysis data;

(xii) where reactions refer to the use of a microwave, it was a Smith Synthesizer Microwave that was used;

(xiii) where a SCX or SCX-2 column is referred to, this means an “ion exchange” extraction cartridge for adsorption of basic compounds, i.e. a polypropylene tube containing a benzenesulphonic acid based strong cation exchange sorbent, used according to the manufacturers instructions obtained from International Sorbent Technologies Limited, Dyffryn Business Park, Hengeod, Mid Glamorgan, UK, CF82 7RJ.

(xiv) the following abbreviations have been used:—

| | | | |
|---|---|---|---|
| DMF | N,N-dimethylformamide | DMA | N,N-dimethylacetamide |
| DCM | dichloromethane | THF | tetrahydrofuran |
| EAA | Enzyme Assay Activity (μM) | m/z | mass spectrometry peak(s) |
| | (In Vitro TTK kinase assay 1) | | |
| EAA2 | Enzyme Assay Activity (μM) | RPHPLC | reverse-phase high- |
| | (In Vitro TTK kinase assay 2) | | performance liquid |
| | | | chromatography |
| r.t. | room temperature | h | hour(s) |
| FCC | flash column chromatography | EtOAc | ethyl acetate |
| DIPEA | N,N-diisopropylethylamine | MeOH | methanol |
| EtOH | ethanol | HATU | O-(7-azabenzotriazol-1-yl)- |
| Pd-on-C | 10% palladium on carbon | | 1,1-3,3-tetramethyluronium |
| SCAA | Spindle checkpoint abrogation assay | | hexafluorophosphate |
| | activity (μM) | | |

### Example 1

### 4-[9-Cyclopentyl-7-methyl-8-oxo-8,9-dihydro-7H-purin-2-yl)-amino]-3-methoxy-N-(1-methylpiperidin-4-yl)benzamide

A solution of 2-chloro-9-cyclopentyl-7-methyl-purin-8-one (Method 4) (0.15 g), 4-amino-3-methoxy-N-(1-methyl-4-piperidyl)benzamide (fragment 4, page 44 of WO 06/018220) (0.16 g) and 4-methylbenzenesulfonic acid (0.28 g) in 4-methyl-2-pentanol (10 mL) was heated at 125° C. for 16 h then at 145° C. for 3 h. The cooled mixture was loaded onto a SCX-2 column, washed with MeOH then eluted with 7N NH₃/MeOH. Concentration in vacuo afforded a gum which was dissolved in 2% MeOH in DCM and then purified through a silica pad eluting with 2-20% MeOH in DCM to afford a yellow gum. Trituration with diethylether containing a small amount of DCM provided the title compound (75 mg, 26%) as an off white solid; ¹H NMR: 1.64 (4H, m), 1.80 (2H, m), 1.95 (4H, m), 2.08 (2H, m), 2.21 (5H, m), 2.84 (2H, m), 3.30 (3H, s), 3.79 (1H, m), 3.96 (3H, s), 4.78 (1H, m), 7.51 (2H, m), 7.88 (1H, s), 8.09 (1H, d), 8.19 (1H, s), 8.40 (1H, d); m/z: MH⁺ 480; EAA: 0.622; EAA2: 0.0646.

### Example 2

### 4-[(9-Cyclopentyl-7-methyl-8-oxo-8,9-dihydro-7H-purin-2-yl)-amino]-3-methylbenzenesulfonamide

A mixture of 4-amino-3-methylbenzenesulfonamide (0.075 g), 4-methylbenzene-sulfonic acid (0.1 g) and 2-chloro-9-cyclopentyl-7-methyl-purin-8-one (Method 4, 0.075 g) in 4-methyl-2-pentanol (1.5 mL) was heated at 200° C. for 5 mins by microwave then cooled to r.t. The reaction was repeated on an identical scale but using 1-butanol (1.5 mL) in place of 4-methyl-2-pentanol. The combined reaction mixtures were purified by SCX, eluting with 2M NH₃ in MeOH to give a coloured gum. The gum was purified by reverse phase basic HPLC to afford the title compound (0.05 g, 21%) as a colourless solid; ¹H NMR: (CDCl₃) 1.69-1.75 (2H, m), 1.96-2.05 (4H, m), 2.26-2.31 (2H, m), 2.40 (3H, s), 3.41 (3H, s), 4.70 (2H, s), 4.79-4.88 (1H, m), 6.98 (1H, d), 7.75 (1H, d), 7.77-7.79 (1H, m), 7.90 (1H, s), 8.54 (1H, d); m/z: MH⁺ 403; EAA: 1.28; EAA2: 0.0166.

### Example 3

### 9-Cyclopentyl-2-{[2-fluoro-4-(methylsulfonyl)phenyl]amino}-7-methyl-7,9-dihydro-8H-purin-8-one

A mixture of 2-fluoro-4-(methylsulfonyl)aniline (0.056 g), 4-methylbenzenesulfonic acid (0.1 g) and 2-chloro-9-cyclopentyl-7-methyl-purin-8-one (Method 4, 0.075 g) in 4-methyl-2-pentanol (1 mL) was heated at 200° C. for 5 mins by microwave then cooled to r.t. The reaction was repeated on an identical scale but using 1-butanol (1.5 mL) in place of 4-methyl-2-pentanol as the solvent. The combined reaction mixtures were purified by SCX, eluting with 2M NH₃ in MeOH to afford a gum. Purification by MPLC on silica, eluting with 2% MeOH in DCM then 10% MeOH in DCM afforded a colourless gum which was triturated with diethylether (1 mL), filtered then dried to provide the title compound (0.02 g, 17%) as a colourless solid; ¹H NMR: (CDCl₃) 1.68-1.80 (2H, m), 1.98-2.06 (4H, m), 2.26-2.32 (2H, m), 3.06 (3H, s), 3.42 (3H, s), 4.79-4.90 (1H, m), 7.43 (1H, d), 7.65-7.69 (1H, m), 7.73 (1H, d), 7.94 (1H, s), 8.84 (1H, t); m/z: MH⁺ 406; EAA2: 0.131; SCAA: 2.3.

The procedure described above for Example 3 was repeated using the appropriate aniline and 2-chloro-9-cyclopentyl-7-methyl-purin-8-one (Method 4) with 4-methyl-2-pentanol as solvent, except that purification was by reverse phase basic chromatography. These procedures provided the compounds of Examples 4 to 28 below:

### Example 4

### 9-Cyclopentyl-2-{[2-methoxy-4-(4-methylpiperazin-1-yl)-phenyl]amino}-7-methyl-7,9-dihydro-8H-purin-8-one

¹H NMR: 1.55-1.68 (2H, m), 1.82-1.94 (4H, m), 2.10-2.18 (2H, m), 2.24 (3H, s), 2.47 (4H, t), 3.11 (4H, t), 3.29 (3H, s), 3.83 (3H, s), 4.68 (1H, quintet), 6.48 (1H, dd), 6.64 (1H, d), 7.56 (1H, s), 7.84 (1H, d), 8.05 (1H, s); m/z: 438 MH⁺; EAA: 0.213; EAA2: 0.0229; prepared using compound 46-3, page 138 of WO 04/080980.

### Example 5

### 9-Cyclopentyl-2-({2-methoxy-4-[(1-methylpiperidin-4-yl)oxy]-phenyl}amino)-7-methyl-7,9-dihydro-8H-purin-8-one

¹H NMR: 1.52-1.70 (4H, m), 1.80-1.98 (6H, m), 2.06-2.24 (7H, m), 2.57-2.69 (2H, m), 3.29 (3H, s), 3.81 (3H, s), 4.28 4.37 (1H, m), 4.69 (1H, quintet), 6.53 (1H, dd), 6.63 (1H, s), 7.63 (1H, s), 7.83 (1H, d), 8.05 (1H, s); m/z: 453 MH⁺; EAA: 0.0717; EAA2: 0.018; preparation: see page 137 of WO 04/080980.

### Example 6

### 9-Cyclopentyl-2-[(2-methoxy-4-morpholin-4-ylphenyl)-amino]-7-methyl-7,9-dihydro-8H-purin-8-one

m/z: 425 MH⁺; EAA: 0.396; EAA2: 0.078; preparation: see example 21 of WO 04/046120.

### Example 7

### N-{4-[(9-Cyclopentyl-7-methyl-8-oxo-8,9-dihydro-7H-purin-2-yl)-amino]-3-methoxyphenyl}methanesulfonamide

m/z: 433 MH⁺; EAA: 0.714; EAA2: 0.0307.

### Example 8

### 4-[(9-Cyclopentyl-7-methyl-8-oxo-8,9-dihydro-7H-purin-2-yl)-amino]-2-fluoro-N-(1-methylpiperidin-4-yl)benzamide

¹H NMR: 1.51-1.61 (2H, m), 1.67-1.79 (4H, m), 1.87-2.06 (6H, m), 2.17 (3H, s), 2.20-2.27 (2H, m), 2.73 (2H, d), 3.67-3.75 (1H, m), 4.75 (1H, quintet), 7.46 (1H, d), 7.53 (1H, t), 7.76-7.79 (1H, m), 7.88 (1H, d), 8.22 (1H, s), 9.81 (1H, s); m/z: 468 MH⁺; EAA: 3.907; EAA2: 0.633; Preparation: see Method 8.

### Example 9

### 4-[(9-Cyclopentyl-7-methyl-8-oxo-8,9-dihydro-7H-purin-2-yl)amino]-3-methoxy-N-methylbenzamide

m/z: 397 MH⁺; EAA: 0.328; EAA2: 0.0322; Preparation: see 128, page 90 of WO 06/021454.

### Example 10

### 9-Cyclopentyl-2-{[2-methoxy-4-(pyrrolidin-1-ylcarbonyl)-phenyl]amino}-7-methyl-7,9-dihydro-8H-purin-8-one

m/z: 437 MH⁺; EAA: 1.05; EAA2: 0.0194; Preparation: see Method 10.

### Example 11

### 4-[(9-Cyclopentyl-7-methyl-8-oxo-8,9-dihydro-7H-purin-2-yl)-amino]benzenesulfonamide

m/z: 389 MH⁺; EAA: 9.65; EAA2: 0.167.

### Example 12

### 9-Cyclopentyl-2-{[4-(1,1-dioxidothiomorpholin-4-yl)-phenyl]-amino}-7-methyl-7,9-dihydro-8H-purin-8-one

m/z: 443 MH⁺; EAA: 1.17; EAA2: 0.0152.

### Example 13

### 9-Cyclopentyl-7-methyl-2-({4-[(4-methylpiperazin-1-yl)-sulfonyl]-phenyl}amino)-7,9-dihydro-8H-purin-8-one

m/z: 472 MH⁺; EAA2: 0.626; SCAA: 3.70.

### Example 14

### 4-[(9-Cyclopentyl-7-methyl-8-oxo-8,9-dihydro-7H-purin-2-yl)-amino]-3-methoxy-N-(9-methyl-9-azabicyclo[3.3.1]non-3-yl)benzamide

m/z: 520 MH⁺; EAA: 1.564; EAA2: 0.0697; Preparation: see Method 11.

### Example 15

### 3-Chloro-4-[(9-cyclopentyl-7-methyl-8-oxo-8,9-dihydro-7H-purin-2-yl)amino]-N-(1-methylpiperidin-4-yl)benzamide

m/z: 485, 487 MH⁺; EAA: 3.23; EAA2: 0.235; Preparation: see Method 12.

### Example 16

### 4-[(9-Cyclopentyl-7-methyl-8-oxo-8,9-dihydro-7H-purin-2-yl)-amino]-3-fluoro-N-(9-methyl-9-azabicyclo[3.3.1]non-3-yl)-benzamide

m/z: 509 MH⁺; EAA: 1.68; EAA2: 0.313; Preparation: see Method 13.

### Example 17

### 4-[(9-Cyclopentyl-7-methyl-8-oxo-8,9-dihydro-7H-purin-2-yl)-amino]-N-(1-ethylpiperidin-4-yl)-2,5-difluorobenzamide

m/z: 501 MH⁺; EAA: 9.24; EAA2: 2.01; Preparation: see Method 15.

### Example 18

### 2-{[2-Chloro-4-(4-methylpiperazin-1-yl)phenyl]amino}-9-cyclopentyl-7-methyl-7,9-dihydro-8H-purin-8-one

¹H NMR: (CDCl₃) 1.62-1.69 (2H, m), 1.90-2.08 (4H, m), 2.23-2.31 (2H, m), 2.36 (3H, s), 2.58 (4H, t), 3.17 (4H, t), 3.38 (3H, s), 4.76-4.85 (1H, m), 6.86 (1H, dd), 6.97 (1H, d), 7.11 (1H, s), 7.84 (1H, s), 8.21 (1H, d); m/z: 442, 444 MH⁺; EAA: 0.249; EAA2: 0.0148; Preparation: see Method 17.

### Example 19

### 9-Cyclopentyl-7-methyl-2-{[4-(4-methylpiperazin-1-yl)-phenyl]-amino}-7,9-dihydro-8H-purin-8-one

¹H NMR: (CDCl₃) 1.68-1.80 (2H, m), 1.98-2.06 (4H, m), 2.26-2.32 (2H, m), 3.06 (3H, s), 3.42 (3H, s), 4.79-4.90 (1H, m), 7.43 (1H, d), 7.65-7.69 (1H, m), 7.73 (1H, d), 7.94 (1H, s), 8.84 (1H, t); m/z: 406 MH⁺; EAA: 0.504; EAA2: 0.0403.

### Example 20

### 9-Cyclopentyl-2-{[2-methoxy-4-(4-methyl-1,4-diazepan-1-yl)-phenyl]amino}-7-methyl-7,9-dihydro-8H-purin-8-one

¹H NMR: 1.62-1.53 (2H, m), 1.95-1.82 (4H, m), 2.17-2.07 (2H, m), 2.27 (3H, s), 2.48-2.44 (3H, m), 2.64-2.59 (3H, m), 3.28 (3H, s), 3.45 (2H, t), 3.54-3.49 (2H, m), 3.78 (3H, s), 4.67 (1H, quintet), 6.24 (1H, d), 6.33 (1H, d), 7.50 (1H, s), 7.58 (1H, d), 7.99 (1H, s); m/z: 452 MH⁺; EAA: 0.249; EAA2: 0.0395; Preparation: see page 17 of WO 06/021548;

### Example 21

### 9-Cyclopentyl-2-{[2-ethoxy-4-(4-methyl-1,4-diazepan-1-yl)-phenyl]amino}-7-methyl-7,9-dihydro-8H-purin-8-one

¹H NMR: 1.30 (3H, t), 1.63-1.53 (2H, m), 1.93-1.82 (4H, m), 2.18-2.07 (2H, m), 2.26 (3H, s), 2.47-2.43 (2H, m), 2.63-2.60 (2H, m), 3.29 (3H, s), 3.43 (3H, t), 3.52-3.47 (3H, m), 4.05 (2H, q), 4.67 (1H, quintet), 6.24 (1H, d), 6.33 (1H, d), 7.45 (1H, s), 7.67 (1H, d), 8.01 (1H, s); m/z: 466 MH⁺; EAA: 0.384; EAA2: 0.0473; Preparation: see Method 19.

### Example 22

### 9-Cyclopentyl-2-({2-methoxy-4-[(1-methylpiperidin-4-yl)-amino]-phenyl}amino)-7-methyl-7,9-dihydro-8H-purin-8-one

¹H NMR: 1.44-1.32 (2H, m), 1.63-1.52 (2H, m), 1.93-1.81 (6H, m), 2.02 (2H, t), 2.14-2.08 (2H, m), 2.17 (3H, s), 2.77-2.67 (3H, m), 3.29 (3H, s), 3.73 (3H, s), 4.66 (1H, quintet), 5.15 (1H, d), 6.13 (1H, d), 6.31 (1H, d), 7.45 (1H, s), 7.50 (1H, d), 7.98 (1H, s); m/z: 452 MH⁺; EAA: 0.317; EAA2: 0.118; Preparation: see page 22 of WO 06/021548.

### Example 23

### 9-Cyclopentyl-2-({4-[4-(dimethylamino)piperidin-1-yl]-2-ethoxyphenyl}amino)-7-methyl-7,9-dihydro-8H-purin-8-one

¹H NMR: 1.33 (3H, t), 1.67-1.45 (6H, m), 1.94-1.80 (6H, m), 2.26-2.10 (8H, m), 2.62 (2H, t), 3.29 (3H, s), 3.63 (2H, d), 4.08 (1H, q), 4.70 (1H, quintet), 6.48 (1H, d), 6.62 (1H, d), 7.51 (1H, s), 7.88 (1H, d), 8.05 (1H, s); m/z: 480 MH⁺; EAA: 0.703; EAA2: 0.0756; Preparation: see Method 21.

### Example 24

### 9-Cyclopentyl-2-({2-methoxy-4-[(1-methylpyrrolidin-3-yl)-oxy]-phenyl}amino)-7-methyl-7,9-dihydro-8H-purin-8-one

¹H NMR: 1.64-1.54 (2H, m), 1.93-1.82 (4H, m), 2.18-2.07 (2H, m), 2.22-2.41 (6H, m), 2.69-2.56 (2H, m), 2.82-2.76 (1H, m), 3.29 (3H, s), 3.80 (3H, s), 4.68 (1H, quintet), 4.89-4.83 (1H, m), 6.43 (1H, d), 6.56 (1H, d), 7.61 (1H, s), 7.81 (1H, d), 8.04 (1H, s); m/z: 439 MH⁺; EAA: 0.151; EAA2: 0.0173; Preparation: see Method 23.

### Example 25

### 9-Cyclopentyl-2-({4-[2-(dimethylamino)ethoxy]-2-ethoxy-phenyl}-amino)-7-methyl-7,9-dihydro-8H-purin-8-one

¹H NMR: 1.34 (3H, t), 1.66-1.56 (2H, m), 1.95-1.83 (4H, m), 2.18-2.09 (2H, m), 2.22 (6H, s), 2.61 (2H, t), 3.29 (3H, s), 4.03 (2H, t), 4.08 (2H, q), 4.70 (1H, quintet), 6.50 (1H, d), 6.61 (1H, d), 7.56 (1H, s), 7.92 (1H, d), 8.06 (1H, s); m/z: 441 MH⁺; EAA: 0.149; EAA2: 0.0289; Preparation: see Method 25.

### Example 26

### 9-Cyclopentyl-2-({2-ethoxy-4-[(1-methylpiperidin-4-yl)-oxy]-phenyl}amino)-7-methyl-7,9-dihydro-8H-purin-8-one

¹H NMR: 1.33 (3H, t), 1.68-1.57 (4H, m), 1.95-1.83 (6H, m), 2.20-2.10 (7H, m), 2.64-2.57 (2H, m), 3.30 (3H, s), 4.07 (2H, q), 4.34-4.27 (1H, m), 4.69 (1H, quintet), 6.52 (1H, d), 6.61 (1H, d), 7.57 (1H, s), 7.89 (1H, d), 8.06 (1H, s); m/z: 467 MH⁺; EAA: 0.0509; EAA2: 0.026; Preparation: see Method 27.

### Example 27

### 9-Cyclopentyl-2-({2-methoxy-4-[2-(4-methylpiperazin-1-yl)-ethyl]phenyl}amino)-7-methyl-7,9-dihydro-8H-purin-8-one

¹H NMR: 1.68-1.57 (2H, m), 1.96-1.85 (4H, m), 2.23-2.11 (5H, m), 2.38-2.27 (4H, m), 2.48-2.40 (5H, m), 2.72-2.65 (3H, m), 3.27 (3H, s), 3.85 (3H, s), 4.71 (1H, quintet), 6.77 (1H, d), 6.91 (1H, d), 7.64 (1H, s), 8.06 (1H, d), 8.10 (1H, s); m/z: 466 MH⁺; EAA: 0.288; EAA2: 0.0241; Preparation: see Method 30.

### Example 28

### 9-Cyclopentyl-2-{[2-methoxy-4-(1-methylpiperidin-4-yl)-phenyl]amino}-7-methyl-7,9-dihydro-8H-purin-8-one

¹H NMR: 1.71-1.49 (7H, m), 1.93-1.77 (6H, m), 2.15-2.04 (5H, m), 2.80 (2H, d), 3.20 (3H, s), 3.79 (3H, s), 4.65 (1H, quintet), 6.72 (1H, d), 6.83 (1H, s), 7.58 (1H, s), 7.99 (1H, d), 8.03 (1H, s); m/z: 437 MH⁺; EAA: 0.0554; EAA2: 0.0143; Preparation: see Method 34.

### Example 29

### 9-Cyclopentyl-7-ethyl-2-{[2-methoxy-4-(4-methylpiperazin-1-yl)-phenyl]amino}-7,9-dihydro-8H-purin-8-one

A solution of 2-chloro-9-cyclopentyl-7-ethyl-purin-8-one (Method 5) (0.1 g), 2-methoxy-4-(4-methylpiperazin-1-yl)aniline (Compound 46-3, page 138 in WO 04/080980) (0.16 g) and 4-toluenesulphonic acid (0.13 g) in 2-propanol (2 mL) was heated at 190° C. for 1 h by microwave. After cooling the mixture was concentrated in vacuo and purified by FCC using 0-5% of (10:1 MeOH:conc. aq. NH₃) in DCM to afford the title compound (0.05 g, 31%) as a pale yellow foam; ¹H NMR: (CDCl₃) 1.34 (3H, t), 1.68 (2H, m), 2.33 (2H, m), 2.36 (3H, s), 2.60 (4H, m), 3.17 (4H, m), 3.87 (2H, q), 3.89 (3H, s), 4.81 (1H, tt), 6.55 (1H, dd), 6.57 (1H, s), 7.30 (1H, s), 7.86 (1H, s), 8.25 (1H, d); m/z: MH⁺ 453; EAA: 0.220; EAA2: 0.0538.

The procedure described for Example 29 was repeated using the appropriate aniline and 2-chloro-9-cyclopentyl-7-ethyl-purin-8-one (Method 5). The compounds thereby synthesized are illustrated below as Examples 30 to 34, and the necessary aniline starting materials indicated. Example 30 was further purified by RPHPLC and Example 32 was further purified by flash chromatography on silica, eluting with 30-50% EtOAc in iso-hexane.

### Example 30

### 9-Cyclopentyl-7-ethyl-2-({2-methoxy-4-[(1-methylpiperidin-4-yl)-oxy]phenyl}amino)-7,9-dihydro-8H-purin-8-one

¹H NMR: (CDCl₃) 1.34 (3H, t), 1.69 (2H, m), 1.85 (2H, m), 2.01 (6H, m), 2.27 (2H, m), 2.31 (3H, s), 2.33 (2H, m), 2.71 (2H, m), 3.88 (5H, m), 4.26 (1H, tt), 4.82 (1H, tt), 6.52 (1H, dd), 6.53 (1H, s), 7.28 (1H, s), 7.87 (1H, s), 8.24 (1H, d); m/z: 467 MH⁺; EAA: 0.0913; EAA2: 0.0108; Preparation: see page 137 of WO 04/080980.

### Example 31

### 4-[(9-Cyclopentyl-7-ethyl-8-oxo-8,9-dihydro-7H-purin-2-yl)-amino]-3-methoxy-N-methylbenzamide

¹H NMR: (CDCl₃) 1.36 (3H, t), 1.72 (2H, m), 2.03 (4H, m), 2.33 (2H, m), 3.03 (3H, d), 3.90 (2H, q), 3.99 (3H, s), 4.84 (1H, tt), 6.08 (1H, d), 7.27 (1H, dd), 7.47 (1H, d), 7.76 (1H, s), 7.93 (1H, s), 8.56 (1H, d); m/z: 411 MH⁺; EAA: 0.371; EAA2: 0.0164; Preparation: see 128, page 90 of WO 06/021454.

### Example 32

### 9-Cyclopentyl-7-ethyl-2-{[2-methoxy-4-(methylsulfonyl)-phenyl]-amino}-7,9-dihydro-8H-purin-8-one

¹H NMR: (CDCl₃) 1.37 (3H, t), 1.73 (2H, m), 2.04 (4H, m), 2.32 (2H, m), 3.06 (3H, s), 3.92 (2H, q), 4.02 (3H, s), 4.85 (1H, tt), 7.39 (1H, d), 7.58 (1H, dd), 7.85 (1H, s), 7.96 (1H, s), 8.76 (1H, d); m/z: 432 MH⁺; EAA: 1.33; EAA2: 0.0245; Preparation: see example 146, page 146 of WO 99/64415.

### Example 33

### 4-[(9-Cyclopentyl-7-ethyl-8-oxo-8,9-dihydro-7H-purin-2-yl)-amino]-3-methoxy-N-(1-methylpiperidin-4-yl)benzamide

¹H NMR: (CDCl₃) 1.69 (3H, m), 1.87 (2H, dddd), 2.01 (6H, m), 2.30 (7H, m), 2.73 (2H, m), 3.89 (3H, s), 4.28 (1H, dddd), 4.76 (2H, s), 4.81 (1H, tt), 6.52 (1H, dd), 6.54 (1H, d), 7.35 (1H, s), 8.03 (1H, s), 8.20 (1H, d); m/z: 495 MH⁺; EAA: 0.398; EAA2: 0.0382; Preparation: see fragment 4, page 44 of WO 06/018220.

### Example 34

### 4-[(9-Cyclopentyl-7-ethyl-8-oxo-8,9-dihydro-7H-purin-2-yl)-amino]benzenesulfonamide

¹H NMR: 1.26 (3H, t), 1.68 (2H, m), 1.97 (4H, m), 2.23 (2H, m), 3.86 (2H, q), 4.76 (1H, tt), 7.13 (2H, s), 7.71 (2H, d), 7.91 (2H, d), 8.28 (1H, s), 9.76 (1H, s); m/z: 403; EAA: 1.04; EAA2: 0.0963.

The procedure described for Example 29 was repeated using the appropriate aniline and 2-(2-chloro-9-cyclopentyl-8-oxo-purin-7-yl)acetonitrile (Method 6) in place of 2-chloro-9-cyclopentyl-7-ethyl-purin-8-one (Method 5) to provide the compounds of Examples 35 to 38. The necessary aniline starting materials can be prepared as indicated. Additional purification for Example 35 and Example 36 was by crystallisation from diethyl ether and for Example 37 by crystallisation from EtOAc/iso-hexane.

### Example 35

### [9-Cyclopentyl-2-({2-methoxy-4-[(1-methylpiperidin-4-yl)-oxy]-phenyl}amino)-8-oxo-8,9-dihydro-7H-purin-7-yl]-acetonitrile

¹H NMR: (CDCl₃) 1.36 (3H, t), 1.60 (2H, m), 1.72 (2H, m), 2.04 (6H, m), 2.17 (2H, ddd), 2.31 (3H, s), 2.33 (2H, m), 2.83 (2H, m), 3.90 (2H, q), 3.99 (3H, s), 4.00 (1H, m), 4.85 (1H, tt), 5.91 (1H, d), 7.26 (1H, dd), 7.45 (1H, d), 7.76 (1H, s), 7.93 (1H, s), 8.56 (1H, d); m/z: 479 MH⁺; EAA: 0.0479; EAA2: 0.00857; Preparation: see page 137 of WO 04/080980.

### Example 36

### (9-Cyclopentyl-2-{[2-methoxy-4-(4-methylpiperazin-1-yl)-phenyl]-amino}-8-oxo-8,9-dihydro-7H-purin-7-yl)acetonitrile

¹H NMR: (CDCl₃) 1.70 (2H, m), 2.01 (4H, m), 2.30 (2H, m), 2.37 (3H, s), 2.61 (4H, m), 3.19 (4H, m), 3.90 (3H, s), 4.75 (2H, s), 4.80 (1H, tt), 6.54 (1H, d), 6.57 (1H, s), 7.37 (1H, s), 8.02 (1H, s), 8.20 (1H, d); m/z: 464 MH⁺; EAA: 0.217; EAA2: 0.0216; Preparation: see compound 46-3, page 138 of WO 04/080980.

### Example 37

### 4-{[7-(Cyanomethyl)-9-cyclopentyl-8-oxo-8,9-dihydro-7H-purin-2-yl]amino}-3-methoxy-N-(1-methylpiperidin-4-yl)-benzamide

m/z: 506 MH⁺; EAA: 0.501; EAA2: 0.0834; Preparation: see Fragment 4, page 44 of WO 06/018220.

### Example 38

### 4-{[7-(Cyanomethyl)-9-cyclopentyl-8-oxo-8,9-dihydro-7H-purin-2-yl]amino}benzenesulfonamide

m/z: 414 MH⁺; EAA: 1.51; EAA2: 0.131.

### Example 39

### 2-Fluoro-4-[(9-isopropyl-7-methyl-8-oxo-8,9-dihydro-7H-purin-2-yl)-amino]-N-(1-methylpiperidin-4-yl)benzamide

4-Amino-2-fluoro-N-(1-methyl-4-piperidyl)benzamide (Method 8, 0.11 g) and 4-methylbenzenesulfonic acid (0.1 g) were added to a stirred mixture of 2-chloro-9-isopropyl-7-methyl-7,9-dihydro-8H-purin-8-one (Method 42, 75 mg) in n-butanol (0.5 mL) and isopropanol (0.5 mL). The resulting mixture was heated to 180° C. for 10 mins in a microwave. The mixture was loaded onto a SCX-3 column and washed with MeOH. The column was eluted with 2M NH₃ in MeOH. Fractions that contained the title compound were concentrated in vacuo. Additional purification by RPHPLC afforded the title compound as a solid (0.09 g, 42%); m/z: MH⁺ 442; EAA2: 2.455.

The procedure described for Example 39 was repeated using the appropriate aniline and 2-chloro-9-isopropyl-7-methyl-7,9-dihydro-8H-purin-8-one (Method 42). The compounds thereby synthesized are illustrated below as Examples 40 to 48.

### Example 40

### 4-[(9-Isopropyl-7-methyl-8-oxo-8,9-dihydro-7H-purin-2-yl)amino]-3-methoxy-N-(1-methylpiperidin-4-yl)benzamide

m/Z: 454 MH⁺; EAA2: 0.338.

### Example 41

### 4-[(9-Isopropyl-7-methyl-8-oxo-8,9-dihydro-7H-purin-2-yl)amino]-3-methoxy-N-methylbenzamide

m/z: 371 MH⁺; EAA2: 7.126.

### Example 42

### 9-Isopropyl-2-({2-methoxy-4-[(1-methylpiperidin-4-yl)oxy]phenyl}-amino)-7-methyl-7,9-dihydro-8H-purin-8-one

m/z: 427 MH⁺; EAA2: 0.0688.

### Example 43

### 9-Isopropyl-2-({2-methoxy-4-[(1-methylpyrrolidin-3-yl)oxy]-phenyl}amino)-7-methyl-7,9-dihydro-8H-purin-8-one

m/z: 413 MH⁺; EAA2: 0.115.

### Example 44

### 9-Isopropyl-2-{[2-methoxy-4-(4-methylpiperazin-1-yl)phenyl]-amino}-7-methyl-7,9-dihydro-8H-purin-8-one

m/z: 412 MH⁺; EAA2: 0.119.

### Example 45

### 2-{[4-(4-Ethylpiperazin-1-yl)-2-methoxyphenyl]amino}-9-isopropyl-7-methyl-7,9-dihydro-8H-purin-8-one

m/z: 426 MH⁺; EAA2: 0.109.

### Example 46

### 2-{[2-Ethoxy-4-(4-methylpiperazin-1-yl)phenyl]amino}-9-isopropyl-7-methyl-7,9-dihydro-8H-purin-8-one

m/z: 426 MH⁺; EAA2: 0.144.

### Example 47

### 9-Isopropyl-2-{[2-methoxy-4-(1-methylpiperidin-4-yl)phenyl]-amino}-7-methyl-7,9-dihydro-8H-purin-8-one

m/z: 411 MH⁺; EAA: 5.65.

### Example 48

### 9-Isopropyl-2-{[2-methoxy-4-(4-methyl-1,4-diazepan-1-yl)phenyl]-amino}-7-methyl-7,9-dihydro-8H-purin-8-one

m/z: 426 MH⁺; EAA2: 0.136.

### Example 49

### 4-[(9-Cyclopentyl-7-methyl-8-oxo-8,9-dihydro-7H-purin-2-yl)-amino]-3-methoxybenzoic acid

p-Toluenesulfonic acid monohydrate (1.42 g) was added to 2-chloro-9-cyclopentyl-7-methyl-purin-8-one (Method 4, 0.95 g) and methyl 4-amino-3-methoxybenzoate (0.68 g) in 4-methyl-2-pentanol (15 mL). The resulting suspension was heated at 160° C. for 1 h in a microwave reactor. The crude mixture was then stirred in 2M aq. NaOH (45 mL) and heated at reflux for 16 h. The mixture was then concentrated in vacuo, EtOH (50 mL) was added and the resulting mixture was refluxed for 1 h. The mixture was then concentrated in vacuo then diluted with water (150 mL) and the resulting mixture was heated to reflux. Acetic acid (20 mL) was added slowly, then the mixture was allowed to cool to r.t. The solid that formed was collected by filtration and then washed with water followed by diethyl ether. The solid was dried over P₂O₅ to provide the title compound (2.59 g, 60%) as a beige solid; ¹H NMR: 1.67 (2H, m), 1.94 (4H, m), 2.20 (2H, m), 3.33 (3H, s), 3.95 (3H, s), 4.75 (1H, m), 7.51 (1H, d), 7.59 (1H, dd), 7.94 (1H, s), 8.20 (1H, s), 8.48 (1H, d); m/z: MH⁺ 384; EAA2: 0.0724.

### Example 50

### 9-Cyclopentyl-2-({2-methoxy-4-[(4-methylpiperazin-1-yl)carbonyl]-phenyl}amino)-7-methyl-7,9-dihydro-8H-purin-8-one

O-Benzotriazol-1-yl-N,N,N′,N′-tetramethyluronium hexafluorophosphate (HBTU) (0.083 g, 0.22 mmol) was added to a suspension of 4-[(9-cyclopentyl-7-methyl-8-oxo-8,9-dihydro-7H-purin-2-yl)amino]-3-methoxybenzoic acid (Example 49, 0.077 g) in triethylamine (0.040 g) and DMA (1.00 mL), at r.t. After 20 mins 1-methylpiperazine (0.030 g) was added and the mixture was stirred for 16 h. The mixture was then diluted with MeOH (1.0 mL) and then 0.5 M aq. NaOH (20 mL). The solid that formed was isolated by filtration. The solid was washed with water, and dried in vacuo over P₂O₅ to provide the title compound (0.072 g, 77%) as a beige solid; ¹H NMR: 1.64 (2H, m), 1.92 (4H, m), 2.17 (2H, m), 2.21 (3H, s), 2.33 (4H, s), 3.30 (3H, s), 3.52 (4H, s), 3.91 (3H, s), 4.74 (1H, m), 6.99 (1H, d), 7.04 (1H, s), 7.84 (1H, s), 8.16 (1H, s), 8.31 (1H, d); m/z: MH⁺ 466; EAA2: 0.0603.

The procedure described for Example 50 was repeated using the appropriate amine and 4-[(9-cyclopentyl-7-methyl-8-oxo-8,9-dihydro-7H-purin-2-yl)amino]-3-methoxybenzoic acid (Method 49), except with additional purification by RPHPLC. The compounds thereby synthesized are listed below as Examples 51 to 69. In the synthesis of Examples 68 and 69 the appropriate pyrrolidine compound was protected by a tert-butoxycarbonyl (BOC) protecting group during the procedure, and after the coupling was achieved the BOC group was removed using standard conditions well-known to those skilled in the art, using trifluoroacetic acid (TFA) and water.

### Example 51

### 9-Cyclopentyl-2-[(2-methoxy-4-{[4-(1-methylpiperidin-4-yl)-piperazin-1-yl]-carbonyl}phenyl)amino]-7-methyl-7,9-dihydro-8H-purin-8-one

¹H NMR: 1.43 (2H, m), 1.65 (4H, m), 1.80-2.00 (6H, m), 2.14 (3H, s), 2.18 (2H, m), 2.77 (2H, m), 3.25-3.40 (8H, m), 3.50 (4H, s), 3.90 (3H, s), 4.74 (1H, m), 6.98 (1H, dd), 7.04 (1H, d), 7.84 (1H, s), 8.16 (1H, s), 8.31 (1H, d); m/z: MH⁺ 549; EAA2: 0.148.

### Example 52

### 4-[(9-Cyclopentyl-7-methyl-8-oxo-8,9-dihydro-7H-purin-2-yl)-amino]-N-[3-(1H-imidazol-1-yl)propyl]-3-methoxybenzamide

¹H NMR: 1.67 (2H, m), 1.98 (6H, m), 2.20 (2H, m), 3.26 (2H, m), 3.33 (3H, s), 3.95 (3H, s), 4.03 (2H, t), 4.75 (1H, m), 6.90 (1H, s), 7.22 (1H, s), 7.51 (2H, m), 7.67 (1H, s), 7.86 (1H, s), 8.19 (1H, s), 8.37 (1H, t), 8.41 (1H, d); m/z: MH⁺ 491; EAA2: 0.0412.

### Example 53

### 4-[(9-Cyclopentyl-7-methyl-8-oxo-8,9-dihydro-7H-purin-2-yl)-amino]-N-[(1-isopropylpyrrolidin-3-yl)methyl]-3-methoxy-N-methylbenzamide

m/z: 522 MH⁺; EAA2: 0.231.

### Example 54

### 4-[(9-Cyclopentyl-7-methyl-8-oxo-8,9-dihydro-7H-purin-2-yl)-amino]-N-[3-(dimethylamino)propyl]-3-methoxy-N-methylbenzamide

¹H NMR: 1.70 (4H, m), 1.93 (4H, m), 2.00-2.30 (10H, m), 2.96 (3H, s), 3.91 (3H, s), 4.74 (1H, m), 6.98 (1H, m), 7.03 (1H, d), 7.83 (1H, s), 8.16 (1H, s), 8.32 (1H, d); m/z: 482 MH⁺; EAA2: 0.0362.

### Example 55

### 9-Cyclopentyl-2-[(4-{[(3R)-3-(dimethylamino)pyrrolidin-1-yl]-carbonyl}-2-methoxyphenyl)amino]-7-methyl-7,9-dihydro-8H-purin-8-one

m/z: 480 MH⁺; EAA2: 0.0175.

### Example 56

### 9-Cyclopentyl-2-({2-methoxy-4-[(4-pyrrolidin-1-ylpiperidin-1-yl)-carbonyl]phenyl}amino)-7-methyl-7,9-dihydro-8H-purin-8-one

m/z: 520 MH⁺; EAA2: 0.0676.

### Example 57

### 9-Cyclopentyl-2-[(2-methoxy-4-{[4-(2-methoxyethyl)piperazin-1-yl]-carbonyl}phenyl)amino]-7-methyl-7,9-dihydro-8H-purin-8-one

m/z: 510 MH⁺; EAA2: 0.0356

### Example 58

### 4-[9-Cyclopentyl-7-methyl-8-oxo-8,9-dihydro-7H-purin-2-yl)-amino]-N-[4-(dimethylamino)cyclohexyl]-3-methoxybenzamide

m/z: 508 MH⁺; EAA2: 0.136.

### Example 59

### 4-[(9-Cyclopentyl-7-methyl-8-oxo-8,9-dihydro-7H-purin-2-yl)-amino]-3-methoxy-N-[1-(2-methoxyethyl)piperidin-4-yl]benzamide

m/z: 524 MH⁺; EAA2: 0.0745.

### Example 60

### 4-[(9-Cyclopentyl-7-methyl-8-oxo-8,9-dihydro-7H-purin-2-yl)-amino]-N-[(1-ethylpyrrolidin-2-yl)methyl]-3-methoxybenzamide

m/z: 494 MH⁺; EAA2: 0.114.

### Example 61

### 4-[(9-Cyclopentyl-7-methyl-8-oxo-8,9-dihydro-7H-purin-2-yl)-amino]-N-[4-(dimethylamino)butyl]-3-methoxybenzamide

m/z: 480; EAA2: 0.0536.

### Example 62

### 4-[(9-Cyclopentyl-7-methyl-8-oxo-8,9-dihydro-7H-purin-2-yl)-amino]-N-[3-(dimethylamino)propyl]-3-methoxybenzamide

m/z: 468 MH⁺; EAA2: 0.0509.

### Example 63

### 4-[(9-Cyclopentyl-7-methyl-8-oxo-8,9-dihydro-7H-purin-2-yl)-amino]-3-methoxy-N-(2-piperidin-1-ylethyl)benzamide

m/z: 494 MH⁺; EAA2: 0.0664.

### Example 64

### 4-[(9-Cyclopentyl-7-methyl-8-oxo-8,9-dihydro-7H-purin-2-yl)-amino]-3-methoxy-N-[2-(4-methylpiperazin-1-yl)ethyl]benzamide

m/z: 509 MH⁺; EAA2: 0.0611.

### Example 65

### 4-[(9-Cyclopentyl-7-methyl-8-oxo-8,9-dihydro-7H-purin-2-yl)-amino]-3-methoxy-N,N-dimethylbenzamide

¹H NMR: 1.63 (2H, m), 1.93 (4H, m), 2.18 (2H, m), 2.99 (6H, s), 3.91 (3H, s), 4.74 (1H, m), 7.01 (1H, m), 7.07 (1H, m), 7.83 (1H, s), 8.16 (1H, s), 8.32 (1H, d); m/z: 411 MH⁺; EAA2: 0.027.

### Example 66

### 4-[(9-Cyclopentyl-7-methyl-8-oxo-8,9-dihydro-7H-purin-2-yl)-amino]-3-methoxy-N-(4-pyrrolidin-1-ylbutyl)benzamide

m/z: 508 MH⁺; EAA2: 0.132.

### Example 67

### 4-[(9-Cyclopentyl-7-methyl-8-oxo-8,9-dihydro-7H-purin-2-yl)-amino]-N-[2-(dimethylamino)ethyl]-3-methoxybenzamide

¹H NMR: 1.67 (2H, m), 1.95 (4H, m), 2.18 (2H, m), 2.25 (6H, s), 2.46 (2H, m), 3.38 (2H, m), 3.95 (3H, s), 4.75 (1H, m), 7.50 (2H, m), 7.86 (1H, s), 8.19 (1H, s), 8.28 (1H, t), 8.40 (1H, d); m/z: 454 MH⁺; EAA2: 0.119.

### Example 68

### 4-[(9-Cyclopentyl-7-methyl-8-oxo-8,9-dihydro-7H-purin-2-yl)-amino]-3-methoxy-N-[(3R)-pyrrolidin-3-yl]benzamide

m/z: 452 MH⁺; EAA2: 0.0653.

### Example 69

### 4-[(9-Cyclopentyl-7-methyl-8-oxo-8,9-dihydro-7H-purin-2-yl)-amino]-3-methoxy-N-[(3S)-pyrrolidin-3-yl]benzamide

¹H NMR: 1.67 (3H, m), 1.96 (5H, m), 2.20 (2H, m), 2.64-3.04 (4H, m), 3.33 (3H, s), 3.96 (3H, s), 4.32 (1H, m), 4.75 (1H, m), 7.51 (2H, m), 7.86 (1H, s), 8.14 (1H, d), 8.19 (1H, s), 8.39 (1H, d); m/z: 452 MH⁺; EAA2: 0.0413.

### Example 70

### 2-{[2-Methoxy-4-(4-methylpiperazin-1-yl)phenyl]amino}-7-methyl-9-piperidin-4-yl-7,9-dihydro-8H-purin-8-one

p-Toluenesulfonic acid monohydrate (0.304 g) was added to a mixture of 2-chloro-7-methyl-9-piperidin-4-yl-7,9-dihydro-8H-purin-8-one 4-methylbenzenesulfonate (Method 38, 0.176 g), and 2-methoxy-4-(4-methylpiperazin-1-yl)aniline (Compound 46-3, page 138 in WO 04/080980, 0.089 g) in 4-methyl-2-pentanol (3 mL). The resulting suspension was heated at 160° C. for 1 h in a microwave. The cooled solution was decanted and the residue was purified by RPHPLC to provide the title compound as a brown gum (0.142 g, 57%); ¹H NMR: 1.94 (2H, m), 2.29 (6H, s), 2.60 (obscured, m), 3.12 (6H, m), 3.35 (obscured, m), 3.85 (3H, s), 4.52 (1H, m), 6.52 (1H, m), 6.66 (1H, d), 7.12 (2H, d), 7.40 (1H, s), 7.48 (2H, d), 8.06 (1H, d), 8.11 (1H, s); m/z: MH⁺ 453; EAA2: 5.36.

The procedure described for Example 2 was repeated using the appropriate aniline and 2-chloro-9-cyclopentyl-7-methyl-purin-8-one (Method 4) with 4-methyl-2-pentanol as solvent under microwave heating for 15 mins, except that purification was by reverse phase basic chromatography or preparative thin layer chromatography. These procedures provided the compounds of Examples 71 to 80 below:

### Example 71

### 2-[(4-Benzoylphenyl)amino]-9-cyclopentyl-7-methyl-7,9-dihydro-8H-purin-8-one

m/z: 414 MH⁺; EAA2: 0.586.

### Example 72

### 2-[(3-Chloro-4-morpholin-4-ylphenyl)amino]-9-cyclopentyl-7-methyl-7,9-dihydro-8H-purin-8-one

m/z: 429 MH⁺; EAA2: 0.027.

### Example 73

### 9-Cyclopentyl-2-{[4-(2-hydroxyethyl)phenyl]amino}-7-methyl-7,9-dihydro-8H-purin-8-one

m/z: 354 MH⁺; EAA2: 0.106.

### Example 74

### 9-Cyclopentyl-2-[(4-isopropoxyphenyl)amino]-7-methyl-7,9-dihydro-8H-purin-8-one

m/z: 368 MH⁺; EAA2: 0.0291.

### Example 75

### 9-Cyclopentyl-7-methyl-2-[(4-phenoxyphenyl)amino]-7,9-dihydro-8H-purin-8-one

m/z: 402 MH⁺; EAA2: 0.523.

### Example 76

### 9-Cyclopentyl-7-methyl-2-{[4-(1,3-oxazol-5-yl)phenyl]amino}-7,9-dihydro-8H-purin-8-one

m/z: 377 MH⁺; EAA2: 0.0183.

### Example 77

### 9-Cyclopentyl-7-methyl-2-[(4-piperidin-1-ylphenyl)amino]-7,9-dihydro-8H-purin-8-one

m/z: 393 MH⁺; EAA2: 0.109.

### Example 78

### 2-[(4-Benzylphenyl)amino]-9-cyclopentyl-7-methyl-7,9-dihydro-8H-purin-8-one

m/z: 400 MH⁺; EAA2: 0.195.

### Example 79

### 9-Cyclopentyl-7-methyl-2-{[4-(1H-pyrazol-1-yl)phenyl]amino}-7,9-dihydro-8H-purin-8-one

m/z: 376 MH⁺; EAA2: 0.0452.

### Example 80

### 9-Cyclopentyl-7-methyl-2-[(4-morpholin-4-ylphenyl)amino]-7,9-dihydro-8H-purin-8-one

m/z: 395 MH⁺; EAA2: 0.14.

### Method 1

### 2-Chloro-N-cyclopentyl-5-nitro-pyrimidin-4-amine

Cyclopentylamine (9.9 mL) in EtOAc (100 mL) was added dropwise over 30 mins to a stirred, cooled (ice-bath) solution of 2,4-dichloro-5-nitropyrimidine (19.4 g) and DIPEA (17.8 mL) in dry EtOAc (100 mL). The mixture was allowed to warm to r.t. and was then stirred for 16 h. The mixture was washed with water, 1M citric acid, sat. aq. NaHCO₃ and was then dried (MgSO₄). Concentration in vacuo afforded the title compound (24.9 g, 100%) as a red brown solid; ¹H NMR: 1.50-1.80 (6H, m), 2.00 (2H, m), 4.50 (1H, m), 8.60 (1H, d), 9.01 (1H, s); m/z: MH⁺ 243, 245 (1×Cl).

### Method 2

### 2-Chloro-N-cyclopentyl-pyrimidine-4,5-diamine

A solution of 2-chloro-N-cyclopentyl-5-nitro-pyrimidin-4-amine (Method 1) (1.95 g) in EtOAc (10 mL) was added dropwise to a stirred suspension of tin (II) chloride dihydrate (7.22 g) in EtOAc (10 mL) heated at 50° C. The rate of addition was controlled to maintain the temperature of the mixture below 60° C. The mixture was then stirred at 60° C. for 1.5 h then cooled in ice and conc. aq. NH₃ added slowly until basic. The solid formed was filtered and washed with EtOAc, the combined filtrate and washings were washed with water, brine, dried (MgSO₄) and the solvent evaporated to give the title compound as a purple gum (1.5 g, 88%); ¹H NMR: 1.48 (2H, m), 1.59 (2H, m), 1.70 (2H, m), 1.98 (2H, m), 4.24 (1H, m), 4.92 (2H, m), 6.63 (1H, d), 7.37 (1H, s); m/z: MH⁺ 213, 215 (1×Cl).

### Method 3

### 2-Chloro-9-cyclopentyl-7H-purin-8-one

Phenyl chloroformate (21.1 g,) was added dropwise over 20 mins to a cooled (ice-bath) suspension of 2-chloro-N-cyclopentyl-pyrimidine-4,5-diamine (Method 2) (19.1 g) and NaHCO₃ (22.7 g) in a mixture of EtOAc (250 mL) and water (100 mL). After stirring for 30 mins the reaction mixture was warmed to r.t. over 30 mins, and was then heated at 70° C. for 1.5 h. After cooling to r.t. EtOAc (300 mL) was added, the organic phase was separated and washed with 1M HCl followed by sat. aq. NaHCO₃. The solution was then dried (MgSO₄) and concentrated in vacuo. Purification by FCC using a gradient of 0-50% EtOAc in DCM afforded the title compound (12.4 g, 57%) as an off-white solid; ¹H NMR: 1.63 (2H, m) 1.93 (4H, m), 2.10 (2H, m), 4.70 (1H, m), 8.11 (1H, s) 11.60 (1H, s); m/z: MH⁺ 237, 239 (1×Cl).

### Method 4

### 2-Chloro-9-cyclopentyl-7-methyl-purin-8-one

Iodomethane (0.86 mL) was added in one portion to a cooled (ice-bath) solution of 2-chloro-9-cyclopentyl-7H-purin-8-one (Method 3) (3.0 g) in DMA (30 mL). NaH (0.55 g) was added portionwise and the resulting mixture was stirred at 5-10° C. for 3 h. Ice was then added cautiously to the mixture followed by water (150 mL) and the mixture was then stirred with iso-hexane (30 mL). The resulting precipitate was collected by filtration, washed with water then iso-hexane and dried under vacuum to afford the title compound (3.15 g, 99%) as a white solid; ¹H NMR: 1.65 (2H, m), 1.93 (4H, m), 2.12 (2H, m), 3.37 (3H, s), 4.73 (1H, m), 8.33 (1H, s); m/z: MH⁺ 253, 255 (1×Cl).

### Method 5

### 2-Chloro-9-cyclopentyl-7-ethyl-purin-8-one

Iodoethane (0.86 g) was added in one portion to cooled (ice-bath) solution of 2-chloro-9-cyclopentyl-7H-purin-8-one (Method 3, 1.2 g) in DMA (10 mL). NaH (0.22 g) was added portionwise and the resulting mixture stirred at 5-10° C. for 3 h, then at 20° C. for a further 16 h. Ice was then added cautiously to the mixture followed by water (50 mL), and the mixture was then stirred with iso-hexane (10 mL). The resulting precipitate was collected by filtration, washed with water then iso-hexane and dried under vacuum to afford the title compound (1.01 g, 75%) as a white solid; ¹H NMR: 1.24 (3H, t), 1.63 (2H, m), 1.93 (4H, m), 2.12 (2H, m), 3.88 (2H, q), 4.74 (1H, m), 8.40 (1H, s); m/z: MH⁺267, 269 (1×Cl).

### Method 6

### 2-(2-Chloro-9-cyclopentyl-8-oxo-purin-7-yl)acetonitrile

2-Bromoacetonitrile (0.66 g) was added in one portion to a cooled (ice-bath) solution of 2-chloro-9-cyclopentyl-7H-purin-8-one (Method 3, 1.2 g) in DMA (10 mL). NaH (0.22 g) was then added portionwise and the resulting mixture stirred at 5-10° C. for 3 h, then at 20° C. for a further 16 h. The mixture was then cooled to 5-10° C. Additional 2-bromoacetonitrile (0.33 g) and NaH (0.11 g) were added then the mixture was stirred at r.t. for a further 3 h. Ice was then added cautiously to the mixture followed by water (50 mL), and the mixture was then stirred with iso-hexane (10 mL). The resulting precipitate was collected by filtration, washed with water then iso-hexane and dried under vacuum to afford the title compound (1.32 g, 95%) as a white solid; ¹H NMR: 1.65 (2H, m), 1.94 (4H, m), 2.12 (2H, m), 4.75 (1H, m), 5.15 (2H, s), 8.49 (1H, s); m/z: MH⁺ 278, 280 (1×Cl).

### Method 7

### 2-Fluoro-N-(1-methyl-4-piperidyl)-4-nitro-benzamide

2-Fluoro-4-nitrobenzoic acid (3 g), 4-amino-1-methylpiperidine (2.03 g), HATU (6.77 g), DIPEA (8.5 mL) and DMF (30 mL) were combined and stirred at r.t. for 18 h. The mixture was then concentrated in vacuo and the resulting residue was partitioned between DCM (200 mL) and water (100 mL) and the phases were separated. The aqueous phase was re-extracted with DCM (100 mL). The combined organic portions were dried (MgSO₄) and concentrated in vacuo. Purification by FCC using a gradient of 0-5% (2M ammonia in MeOH) in DCM afforded the title compound (2.67 g, 59%) as a yellow solid; ¹H NMR: 1.53 (2H, m), 1.80 (2H, m), 2.26 (5H, m), 2.85 (2H, m), 3.74 (1H, m), 7.72 (1H, m), 8.06 (1H, m), 8.12 (1H, m), 8.54 (1H, d); m/z: MH⁺ 282.

### Method 8

### 4-Amino-2-fluoro-N-(1-methyl-4-piperidyl)benzamide

2-Fluoro-N-(1-methyl-4-piperidyl)-4-nitro-benzamide (Method 7) (1.62 g), Pd-on-C (0.16 g) and MeOH (50 mL) were combined and stirred at 25° C. under hydrogen at 5 bar pressure for 16 h. The catalyst was filtered and the filtrate was concentrated to afford a brown solid which was triturated with 5% MeOH in DCM. The resulting precipitate was collected by filtration and dried under vacuum to afford the title compound (0.56 g, 39%) as a light-brown solid; ¹H NMR: 1.74 (2H, m), 1.99 (2H, m), 2.76 (3H, s), 3.09 (2H, m), 3.42 (2H, m), 3.97 (1H, m), 5.89 (2H, s), 6.30 (1H, m), 6.39 (1H, m), 7.37 (1H, m), 7.67 (1H, s), 9.11 (1H, s); m/z: MH⁺ 252.

### Method 9

### (3-Methoxy-4-nitro-phenyl)-pyrrolidin-1-yl-methanone

Pyrrolidine (2.45 mL) in THF (5 mL) was added over 10 mins to a stirred, cooled (ice-bath) solution of 3-methoxy-4-nitro-benzoyl chloride (5.3 g) and DIPEA (5.14 mL) in THF (25 mL). The mixture was warmed to r.t. and stirred for 16 h and was then concentrated in vacuo. The resulting residue was diluted with EtOAc (150 mL) and washed with water (3×20 mL) then brine (20 mL). The solution was dried (MgSO₄) and concentrated to afford title compound (5.9 g, 96%) as a brown solid after standing; ¹H NMR: (300 MHz, CDCl₃) 1.90-2.04 (4H, m), 3.40 (2H, t), 3.66 (2H, t), 3.99 (3H, s), 7.10-7.14 (1H, dd), 7.26 (1H, d), 7.85 (1H, d); m/z: MH⁺ 251.

### Method 10

### (4-Amino-3-methoxy-phenyl)-pyrrolidin-1-yl-methanone

Pd-on-C (0.58 g) was added to a solution of (3-methoxy-4-nitro-phenyl)-pyrrolidin-1-yl-methanone (Method 9) (5.8 g) in EtOH (150 mL) and the resulting mixture was stirred under a hydrogen atmosphere at 5 bar pressure for 16 h. The catalyst was removed by filtration, washed with EtOH and the filtrate was concentrated in vacuo to afford the title compound (5.9 g, ˜100%) as a brown gum; ¹H NMR: (300 MHz, CDCl₃) 1.90 (4H, m), 3.58 (4H, m), 3.87 (3H, s), 4.45 (4H, m), 6.64 (1H, d), 7.01 (1H, dd), 7.09 (1H, d); m/z: MH⁺ 221.

### Method 11

### 4-Amino-3-methoxy-N-[(3-endo)-9-methyl-9-azabicyclo[3.3.1]non-3-yl]-benzamide

HATU (12.55 g) was added in portions to a cooled (ice-bath) mixture of 4-amino-3-methoxybenzoic acid (5.02 g), endo-9-methyl-9-azabicyclo[3.3.1]nonane-3-one (5.1 g) and DIPEA (10.4 mL) in DMF (150 mL). The reaction mixture was stirred at r.t. for 18 h then the solvent was removed by evaporation. The residue was partitioned between EtOAc (200 mL) and sat. aq. Na₂CO₃ (3×50 mL) then the phases were separated. The organic portion was washed with brine (3×50 mL), dried (MgSO₄) and concentrated in vacuo to afford an oil (14 g). The oil was purified by SCX-2, washing with water then MeOH then eluting with 3.5M NH₃-MeOH to give the a semi-solid material. Trituration with diethyl ether afforded the title compound (4.84 g, 53%) as a brown solid; ¹H NMR: 0.88-0.96 (1H, d), 1.38-1.50 (3H, m), 1.86-1.96 (2H, m), 2.00-2.10 (1H, m), 2.10-2.20 (2H, m), 2.42 (3H, s), 2.92-3.00 (2H, d), 3.82 (3H, s), 4.22-4.38 (1H, m), 5.17 (2H, s), 6.58-6.62 (1H, d), 7.28 (1H, s); 7.30 (1H, s), 7.60-7.64 (1H, d); m/z: MH⁺ 304.

### Method 12

### 4-Amino-3-chloro-N-(1-methyl-4-piperidyl)benzamide

HATU (6.3 g) was added in portions to a cooled (ice-bath) mixture of 4-amino-3-chlorobenzoic acid (2.57 g), 4-amino-N-methyl-piperidine (1.88 g) and DIPEA (5.2 mL) in DMF (50 mL). The mixture was stirred at r.t. for 18 h and was then concentrated in vacuo. The residue was diluted with sat. aq. NaHCO₃ (100 mL), and extracted with EtOAc (4×50 mL). The combined organic portions were washed with brine (2×75 mL), dried (MgSO₄) and concentrated in vacuo to afford a gum. Trituration with diethylether afforded the title compound (1.78 g, 44% yield) as a light brown solid; ¹H NMR: 1.57-1.70 (2H, m), 1.77-1.90 (2H, m), 2.40 (3H, s), 3.00-3.10 (2H, d), 3.15-3.50 (4H, m), 3.75-3.88 (1H, m), 5.85 (2H, s), 6.75-6.80 (1H, d), 7.55-7.60 (1H, dd), 7.75 (1H, s), 7.94-8.00 (1H, d); m/z: MH⁺ 268.

### Method 13

### 4-Amino-3-fluoro-N-[(3-endo)-9-methyl-9-azabicyclo[3.3.1]non-3-yl]-benzamide

4-Amino-3-fluorobenzoic acid (5.0 g), HATU (13.5 g) and DIPEA (18.5 mL) were stirred together in anhydrous DMA (100 mL) for 25 mins. endo-9-methyl-9-azabicyclo[3.3.1]-nonane-3-one (5.5 g) was added and the mixture was stirred at r.t. for 16 h. The solvent was removed in vacuo and the residue was dissolved in MeOH and semi-purified by SCX-2 washing with MeOH and eluting with 2M NH₃/MeOH. Further purification by FCC using 0-10% (2M NH₃/MeOH) in DCM afforded the title compound (5.7 g, 61%) as a white solid after trituration with EtOAc; ¹H NMR: 0.91 (m, 2H), 1.42 (m, 3H), 1.90 (m, 4H), 2.03 (m, 1H), 2.14 (m, 3H), 2.40 (s, 3H), 2.96 (m, 2H), 4.27 (m, 1H), 5.60 (bs, 2H), 6.74 (m, 1H), 7.46 (m, 1H), 7.53 (m, 1H), 7.70 (d, 1H); m/z: MH⁺ 292.

### Method 14

### 2,5-Difluoro-N-(1-methyl-4-piperidyl)-4-nitro-benzamide

A mixture of 1,5-difluoro-4-nitrobenzoic acid (1 g), 4-amino-1-methylpiperidine (0.62 g), HATU (2.05 g), DIPEA (2.57 mL) and DMF (10 mL) was stirred at r.t. for 18 h and was then concentrated in vacuo. The resultant material partitioned between DCM and 2M aq HCl, both phases were loaded onto a SCX-2 column and washed with MeOH then eluted with 2M NH₃ in MeOH. The material obtained was purified by FCC using a gradient of 0-5% (2M NH₃ in MeOH) in DCM to afford the title compound (0.63 g, 43%) as a yellow solid; ¹H NMR: 1.53 (m, 2H), 1.80 (m, 2H), 1.98 (m, 2H), 2.16 (s, 3H), 2.73 (m, 2H), 3.70 (m, 1H), 7.79 (m, 1H), 8.21 (m, 1H), 8.59 (d, 1H); m/z: MH⁺ 300.

### Method 15

### 4-Amino-2,5-difluoro-N-(1-methyl-4-piperidyl)benzamide

2,5-Difluoro-N-(1-methyl-4-piperidyl)-4-nitro-benzamide (Method 14, 0.63 g), Pd-on-C (0.07 g) and MeOH (50 mL) were combined and stirred at 25° C. under a hydrogen atmosphere at 3 bar pressure for 16 h. The catalyst was removed by filtration and the filtrate was concentrated in vacuo to afford the title compound (0.55 g, 97%) as a yellow solid; ¹H NMR: (CDCl₃) 1.58 (m, 2H), 2.03 (m, 2H), 2.18 (m, 2H), 2.30 (s, 3H), 2.78 (m, 2H), 3.99 (m, 1H), 4.14 (s, 2H), 6.47 (m, 2H), 7.71 (m, 1H); m/z: with 270.

### Method 16

### 1-(3-Chloro-4-nitro-phenyl)-4-methyl-piperazine

A solution of 2-chloro-4-fluoronitrobenzene (4.3 g), 1-methylpiperazine (2.99 mL) and DIPEA (5.55 mL) in THF (100 mL) was heated at reflux for 16 h. The mixture was then concentrated in vacuo and the residue was purified by FCC eluting with DCM initially then using 2.5-10% MeOH in DCM. The title compound was thus obtained as a yellow solid; m/z: MH⁺ 256.

### Method 17

### 2-Chloro-4-(4-methylpiperazin-1-yl)aniline

A mixture of 1-(3-chloro-4-nitro-phenyl)-4-methyl-piperazine (Method 16, 6.2 g), glacial acetic acid (200 mL) and iron powder (6.77 g) was stirred at r.t. for 1 h then at 75° C. for 1 h. The mixture was then concentrated in vacuo. The resulting residue was mixed with water and filtered. The filtrate was then basified to pH 12 and filtered through diatomaceous earth, washing with DCM and MeOH. The filtrate was extracted with DCM and washed with brine. Purification by FCC eluting with DCM initially followed by 2-20% MeOH in DCM afforded the title compound (4.4 g, 80%) as a brown oil; ¹H NMR: 2.20 (3H, s), 2.42 (4H, m), 2.94 (4H, m), 4.78 (2H, s), 6.72 (2H, m), 6.78 (2H, s); m/z: MH⁺ 226.

### Method 18

### 1-(3-Ethoxy-4-nitro-phenyl)-4-methyl-1,4-diazepane

1-Methylhomopiperazine (2.82 mL) was added to a stirred solution of 2-ethoxy-4-fluoro-1-nitrobenzene (3.5 g) and DIPEA (6.54 mL) in DMA (17.5 mL). The mixture was heated at 100° C. for 4 h then diluted with water (75 mL) and extracted with DCM (3×150 mL). The combined organic portions were washed with brine (3×75 mL), dried (Na₂SO₄) and concentrated in vacuo to afford the title compound (5.0 g, 95%) as a gum; ¹H NMR: 1.37 (3H, t), 1.94-1.87 (2H, m), 1.96 (3H, s), 2.67 (2H, t), 3.57 (2H, t), 3.66-3.63 (2H, m), 4.18 (2H, q), 6.27 (1H, d), 6.42 (1H, dd), 7.87 (1H, d); m/z: MH⁺ 280.

### Method 19

### 2-Ethoxy-4-(4-methyl-1,4-diazepan-1-yl)aniline

A suspension of 1-(3-ethoxy-4-nitrophenyl)-4-methyl-1,4-diazepane (Method 18, 5 g) and Pd-on-C (0.25 g) in EtOH (50 mL) was stirred under a hydrogen atmosphere for 16 h. The mixture was then filtered and the filtrate was purified by SCX eluting with 7M NH₃/MeOH to afford the title compound (3.76 g, 84%) as a brown gum; ¹H NMR: 1.25 (3H, t), 1.79 (2H, quintet), 2.19 (3H, s), 2.41-2.37 (2H, m), 2.54-2.50 (2H, m), 3.34-3.16 (6H, m), 4.02-3.83 (2H, m), 6.02 (1H, d), 6.16 (1H, s), 6.44 (1H, d); m/z: MH⁺ 250.

### Method 20

### 1-(3-Ethoxy-4-nitrophenyl)-N,N-dimethylpiperidin-4-amine

4-(Dimethylamino)piperidine (2.89 g) was added to a stirred solution of 2-ethoxy-4-fluoro-1-nitrobenzene (3.79 g) and DIPEA (7.1 mL) in DMA (17.5 mL). The mixture was then heated to 100° C. for 4 h. The mixture was then concentrated in vacuo and purified by SCX, eluting with 7M NH₃/MeOH to afford the title compound (6.10 g, 102%) as a yellow solid; ¹H NMR: 1.39 (5H, m), 1.82 (2H, d), 2.19 (6H, s), 2.36 (1H, m), 2.96 (2H, t), 4.00 (2H, d), 4.19 (2H, q), 6.50 (1H, dd), 6.58 (1H, d), 7.86 (1H, d); m/z: MH⁺ 294.

### Method 21

### 1-(4-Amino-3-ethoxyphenyl)-N,N-dimethylpiperidin-4-amine

1-(3-Ethoxy-4-nitrophenyl)-N,N-dimethylpiperidin-4-amine (Method 20, 6 g) and Pd-on-C (0.25 g) in EtOH were stirred under a hydrogen atmosphere at 1 bar pressure at r.t. for 16 h. The mixture was then filtered and the filtrate was concentrated in vacuo to afford the title compound (5.2 g, 97%) as a purple gum; ¹H NMR: 1.06 (2H, t), 1.32 (3H, t), 1.54-1.42 (2H, m), 1.80 (2H, d), 2.16-2.07 (1H, m), 2.19 (6H, s), 3.50-3.36 (2H, m), 3.97 (2H, q), 4.23-4.10 (2H, m), 6.30 (1H, d), 6.47 (1H, d), 6.52 (1H, d); m/z: MH⁺ 264.

### Method 22

### 3-(3-Methoxy-4-nitro-phenoxy)-1-methyl-pyrrolidine

1-Methylpyrrolidin-3-ol (4.96 g) and tetra-n-butylammonium bromide (2.26 g) were added to a stirred mixture of 4-fluoro-2-methoxy-1-nitrobenzene (6.00 g) in toluene (25 mL) and KOH (5.90 g) in water (25 mL). The mixture was heated at 60° C. for 18 h and was then diluted with ice-water (250 mL), EtOAc (400 mL) and toluene (100 mL). The phases were separated. The organic portion was washed with water (200 mL), sat. brine and was then dried (MgSO₄). Concentration in vacuo and purification by FCC using 0-10% MeOH in DCM afforded the title compound (7.70 g, 87%) as a yellow gum; ¹H NMR: 1.78 (1H, m), 2.26 (3H, s), 2.35 (2H, m), 2.69 (2H, m), 2.77 (1H, m), 3.91 (3H, s), 5.04 (1H, m), 6.62 (1H, dd), 6.72 (1H, d), 7.95 (1H, d); m/z: MH⁺ 253.

### Method 23

### 2-Methoxy-4-(1-methylpyrrolidin-3-yl)oxy-aniline

A suspension of 3-(3-methoxy-4-nitrophenoxy)-1-methylpyrrolidine (Method 22, 7.6 g) and Pd-on-C (0.70 g) in EtOH (150 mL) was stirred under a hydrogen atmosphere for 4 h. The mixture was then filtered and the filtrate was concentrated in vacuo to afford the title compound (6.50 g, 97%) as a pale red liquid; ¹H NMR: 1.72 (1H, m), 2.19 (1H, m), 2.24 (3H, s), 2.35 (1H, m), 2.51 (1H, m), 2.60 (1H, m), 2.73 (1H, m), 3.73 (3H, s), 4.23 (2H, s), 4.71 (1H, m), 6.21 (1H, dd), 6.39 (1H, d), 6.52 (1H, d); m/z: MH⁺ 223.

### Method 24

### 2-(3-Ethoxy-4-nitro-phenoxy)-N,N-dimethyl-ethanamine

N,N-Dimethylethanolamine (5.02 mL) was added a mixture of 2-ethoxy-4-fluoro-1-nitrobenzene (4.63 g) and tetra-n-butylammonium bromide (1.61 g) in toluene (20 mL) and 25% w/v aq. KOH (20 mL). The mixture was heated to 80° C. for 16 h and was then poured onto ice water (50 mL). The resulting mixture was extracted with EtOAc (3×100 mL). The combined organic portions were washed with water (2×50 mL) and brine (2×50 mL), dried (MgSO₄) and concentrated in vacuo. Purification by FCC using 5-10% (2M NH₃/MeOH) in DCM afforded the title compound (3.50 g, 55%) as a yellow gum; ¹H NMR: 1.35 (3H, t), 2.23 (6H, s), 2.65 (2H, t), 4.20 (4H, m), 6.66 (1H, dd), 6.79 (1H, d), 7.92 (1H, d); m/z: MH⁺ 255.

### Method 25

### 4-(2-Dimethylaminoethoxy)-2-ethoxy-aniline

A suspension of 2-(3-ethoxy-4-nitrophenoxy)-N,N-dimethylethanamine (Method 24, 3.5 g) and Pd-on-C (0.25 g) in EtOH (35 mL) was stirred under a hydrogen atmosphere for 16 h. The mixture was then filtered and the filtrate was concentrated in vacuo to afford the title compound (3.0 g, 97%) as a purple solid; ¹H NMR: 1.33 (3H, t), 2.21 (6H, s), 2.57 (2H, t), 3.24-3.34 (br s, 2H), 3.91 (2H, t), 3.98 (2H, q), 6.29 (1H, d), 6.44 (1H, d), 6.54 (1H, d); m/z: MH⁺ 225.

### Method 26

### 4-(3-Ethoxy-4-nitro-phenoxy)-1-methyl-piperidine

The title compound was prepared following a similar procedure to Method 24 by using N-methyl-4-piperidinol in place of N,N-dimethylethanolamine to give the title compound (4.72 g, 67%) as a yellow gum; ¹H NMR: 1.35 (3H, t), 1.71-1.62 (2H, m), 2.00-1.91 (2H, m), 2.20 (5H, m), 2.61 (2H, m), 4.20 (2H, q), 4.58 (1H, sep.), 6.68 (1H, dd), 6.76 (1H, d), 7.90 (1H, d); m/z: MH⁺ 281.

### Method 27

### 2-Ethoxy-4-[(1-methyl-4-piperidyl)oxy]aniline

The title compound was prepared following a similar procedure to Method 25 by exchanging 2-(3-ethoxy-4-nitrophenoxy)-N,N-dimethylethanamine (Method 24) for 4-(3-ethoxy-4-nitro-phenoxy)-1-methyl-piperidine (Method 26) to provide the title compound (3.94 g, 93%) as a purple gum; ¹H NMR: 1.32 (3H, t), 1.62-1.52 (2H, m), 1.89-1.81 (2H, m), 2.20-2.06 (5H, m), 2.62-2.55 (2H, m), 3.96 (2H, q), 4.12-4.03 (1H, m), 4.21 (2H, s), 6.33-6.28 (1H, m), 6.43 (1H, d), 6.53 (1H, d); m/z: MH⁺ 251.

### Method 28

### 1-[(E)-2-(3-Methoxy-4-nitro-phenyl)ethenyl]-4-methyl-piperazine

2-Methoxy-4-methyl-1-nitro-benzene (12 g) was dissolved in N,N,N′,N′-tetramethyl-1-[(2-methylpropan-2-yl)oxy]methanediamine (Bredereck's reagent, 30 mL) and the mixture was heated to 100° C. for 18 h. The mixture was concentrated in vacuo and the residue was dissolved in iPrOH (30 mL). N-methylpiperazine (14.4 g) was added then the mixture was heated at 110° C. for 6 h. The mixture was then concentrated in vacuo and the residue was triturated with DME (220 mL). The resulting solid was filtered, washed with diethyl ether and dried. The solid was recrystallised from DME, filtered and washed with cold DME and diethyl ether. The filtrate was concentrated to precipitate a second batch of product which was combined with the initial batch to afford the title compound (5.34 g, 27%) as a solid;

¹H NMR: (300 MHz) 2.21 (3H, s), 2.36 (4H, t), 3.22 (4H, t), 3.89 (3H, s), 5.37 (1H, d), 6.85 (1H, dd), 6.97 (1H, d), 7.34 (1H, d), 7.76 (1H, d).

### Method 29

### 1-[2-(3-Methoxy-4-nitro-phenyl)ethyl]-4-methyl-piperazine

Sodium triacetoxyborohydride (3.23 g) was added to a stirred solution of 1-[(E)-2-(3-methoxy-4-nitro-phenyl)ethenyl]-4-methyl-piperazine (Method 28, 2.77 g) in DME (50 mL) and glacial acetic acid (2.6 mL). After 3 h the mixture was concentrated in vacuo and aq. 2M Na₂CO₃ was added. The phases were separated and the aqueous portion was extracted with EtOAc. The combined organic portions were washed with water then brine and dried (MgSO₄). Purification by SCX-2 column, washing with water and MeOH then eluting with 7M NH₃/MeOH afforded the title compound (2.2 g, 79%) as a solid; ¹H NMR: (300 MHz) 2.17 (3H, s), 2.35 (4H, s), 2.48 (4H, s), 2.55 (2H, t), 2.81 (2H, t), 3.91 (3H, s), 6.97 (1H, dd), 7.25 (1H, dd), 7.79 (1H, d); m/z: MH⁺ 280.

### Method 30

### 2-Methoxy-4-[2-(4-methylpiperazin-1-yl)ethyl]aniline

A suspension of 1-[2-(3-methoxy-4-nitro-phenyl)ethyl]-4-methyl-piperazine (Method 29, 2.2 g) and Pd-on-C (0.3 g) in EtOAc (50 mL) and EtOH (50 mL) was stirred under a hydrogen atmosphere for 4 h. The mixture was then filtered and concentrated in vacuo to afford the title compound (1.7 g, 87%) as a colourless oil; ¹H NMR: (300 MHz) 2.17 (3H, s), 2.24-2.63 (12H, m), 3.78 (3H, s), 4.54 (2H, s), 6.52 (2H, m), 6.70 (1H, s); m/z: MH⁺ 250.

### Method 31

### Benzyl 4-[3-methoxy-4-[(2-methylpropan-2-yl)oxycarbonyl-amino]phenyl]-3,6-dihydro-2H-pyridine-1-carboxylate

Pd(PPh₃)₄ (0.66 g) was added to benzyl 4-(trifluoromethylsulfonyloxy)-3,6-dihydro-2H-pyridine-1-carboxylate (Tetrahedron Lett. 2000, 41(19), 3705) (17.1 g) and 4-(tert-butoxycarbonylamino)-3-methoxyphenyl-boronic acid pinacol ester (in Example 1 of WO 00/017202, page 67) (10.2 g) in a mixture of DME (210 mL) and sat.aq. NaHCO₃ (210 mL). The mixture was heated to 80° C. for 16 h then cooled and diluted with water (200 mL). The phases were separated and the aqueous phase was extracted with EtOAc (1×250 mL, 1×350 mL). The combined organic portions were dried (Na₂SO₄) and concentrated in vacuo. Purification by FCC using 20:80 to 30:70 EtOAc-isohexane, afforded the title compound (11.81 g, 95%) as a pale yellow liquid; ¹H NMR: 1.47 (9H, s), 2.46-2.48 (2H, m), 3.61-3.65 (2H, m), 3.85 (3H, s), 4.08-4.12 (2H, m), 5.13 (2H, s), 6.13-6.17 (1H, m), 6.96-6.98 (1H, m), 7.05 (1H, d), 7.32-7.40 (5H, m), 7.68 (1H, d), 7.85 (1H, s); m/z: MH⁺ 439.

### Method 32

### tert-Butyl N-[2-methoxy-4-(4-piperidyl)phenyl]carbamate

A suspension of benzyl 4-[3-methoxy-4-[(2-methylpropan-2-yl)oxycarbonylamino]-phenyl]-3,6-dihydro-2H-pyridine-1-carboxylate (Method 31, 11.8 g) and Pd-on-C (0.1 g) in EtOH (150 mL) was heated at 100° C. under a hydrogen atmosphere at 10 bar for 20 h. The mixture was filtered and concentrated in vacuo to afford the title compound (8.49 g, 100%) as a colourless liquid; ¹H NMR: 1.46 (9H, s), 1.61-1.65 (2H, m), 1.74-1.78 (2H, m), 2.60-2.74 (3H, m), 3.12-3.16 (2H, m), 3.81 (3H, s), 6.74-6.76 (1H, m), 6.85 (1H, d), 7.56 (1H, d), 7.78 (1H, s); m/z: MH⁺ 307.

### Method 33

### tert-Butyl N-[2-methoxy-4-(1-methyl-4-piperidyl)phenyl]carbamate

Formaldehyde (37% wt. in water) (2.3 mL) then sodium triacetoxyborohydride (7.04 g) was added to a stirred solution of tert-butyl N-[2-methoxy-4-(4-piperidyl)-phenyl]carbamate (Method 32, 7.83 g) in DCM (200 mL). After 16 h DCM (300 mL) was added and phases were separated. The organic portion was washed with sat. aq. NaHCO₃ (2×500 mL), dried (Na₂SO₄) and concentrated in vacuo. Purification by FCC using 2-4% (7N NH₃/MeOH) in DCM afforded the title compound (6.0 g, 73%) as a colourless liquid, which crystallised on standing to give a white solid; ¹H NMR: 1.46 (9H, s), 1.64-1.73 (4H, m), 1.92-1.98 (2H, m), 2.20 (3H, s), 2.40-2.43 (1H, m), 2.84-2.88 (2H, d), 3.81 (3H, s), 6.74-6.77 (1H, m), 6.87 (1H, d), 7.54 (1H, d), 7.76 (1H, s); m/z: MH⁺ 321.

### Method 34

### 2-Methoxy-4-(1-methyl-4-piperidyl)aniline

4.0M HCl in dioxane (100 mL) was added to a solution of tert-butyl N-[2-methoxy-4-(1-methyl-4-piperidyl)phenyl]carbamate (Method 33, 5.99 g) in MeOH (100 mL) and the mixture was stirred for 20 h. The mixture was then concentrated in vacuo and the resulting residue was dissolved in MeOH and water and purified by SCX-2, washing with MeOH, then eluting with 7N NH₃/MeOH to afford the title compound (4.21 g, 70%) as a yellow solid; ¹H NMR: 1.71-1.80 (4H, m), 2.34-2.46 (6H, m), 3.06-3.10 (2H, m), 3.76 (3H, s), 4.51 (2H, br s), 6.53-6.58 (2H, m), 6.67 (1H, d); m/z: MH⁺ 221.

### Method 35

### tert-Butyl 4-(2-chloro-5-nitropyrimidin-4-ylamino)piperidine-1-carboxylate

A solution of tert-buty 4-aminopiperidine-1-carboxylate (5.01 g) in EtOAc (25 mL) was added dropwise to a stirred solution of 2,4-dichloro-5-nitropyrimidine (4.85 g) and DIPEA (3.30 g) in EtOAc (75 mL) at 5° C., over a period of 30 mins. The resulting mixture was stirred at r.t. for 16 h. The mixture was then washed sequentially with water, 1M citric acid, and then sat. NaHCO₃ solution. The organic phase was dried (MgSO₄), filtered and concentrated in vacuo to provide the title compound (8.9 g, 99%) as a yellow solid;

¹H NMR: 1.41 (9H, s), 1.61-1.74 (2H, m), 1.75-1.88 (2H, m), 2.77-3.00 (2H, m), 3.93-3.97 (2H, m), 4.33 (1H, m), 8.67 (1H, d), 9.03 (1H, s); m/z: 356 and 358 (1×Cl).

### Method 36

### tert-Butyl 4-[(5-amino-2-chloropyrimidin-4-yl)amino]piperidine-1-carboxylate

A solution of tert-butyl 4-(2-chloro-5-nitropyrimidin-4-ylamino)piperidine-1-carboxylate (Method 35, 5.7 g) in EtOAc (100 mL) was added dropwise to a mixture of tin (II) chloride dihydrate (14.38 g) in EtOAc (20 mL), while maintaining the temperature at 60° C. The mixture was stirred at 60° C. for 1 h. The mixture was then cooled using ice and then basified by dropwise addition of cone. aq. ammonia. The precipitate that formed was filtered and washed with EtOAc. The filtrate was then washed with water, dried (MgSO₄) and concentrated in vacuo to provide the title compound (1.30 g, 25%) as a gum; ¹H NMR: 1.34 (2H, m), 1.42 (9H, s), 1.90 (2H, m), 2.90 (2H, m), 3.92 (2H, m), 4.04 (1H, m), 4.91 (2H, s), 6.60 (1H, d), 7.40 (1H, s); m/z: MH⁺ 328 and 330 (1×Cl).

### Method 37

### 2-Chloro-N⁴-piperidin-4-ylpyrimidine-4,5-diamine

A solution of phenyl chloroformate (0.75 mL) in EtOAc (5 mL) was added dropwise to a cooled (ice bath) mixture of tert-butyl 4-[(5-amino-2-chloropyrimidin-4-yl)amino]-piperidine-1-carboxylate (Method 36, 1.30 g) in EtOAc (20 mL) and NaHCO₃ (1.0 g) in water (10 mL). The reaction mixture was warmed to r.t. over 1 h., then the temperature was increased to 70° C. for a further 2 h. After cooling, the mixture was cooled, diluted with EtOAc (100 mL), and washed with water, and then with saturated brine. The organic phase was dried (MgSO₄), filtered and evaporated to provide crude material that was purified by FCC, eluting with 0 to 50% EtOAc in DCM to provide the title compound as a purple solid; (0.80 g, 57%); ¹H NMR: 1.43 (9H, m), 1.74 (2H, m), 2.27 (2H, m), 2.87 (2H, m), 4.08 (2H, m), 4.38 (1H, m), 8.13 (1H, s); m/z: 352 and 354 (1×Cl).

### Method 38

### 2-Chloro-7-methyl-9-piperidin-4-yl-7,9-dihydro-8H-purin-8-one 4-methylbenzenesulfonate

Iodomethane (0.16 mL) was added in one portion to a cooled solution (ice bath) of 2-chloro-N⁴-piperidin-4-ylpyrimidine-4,5-diamine (Method 37, 0.8 g) in DMA (5 mL) under an inert atmosphere. NaH (0.1 g) was added portionwise then the mixture was stirred at 5-10° C. for 3 h. The mixture was then quenched cautiously with ice, and then water (25 mL). Iso-hexane (25 mL) was added. A precipitate was produced. The precipitate was collected by filtration, and was then washed with water then iso-hexane, and then dried in vacuo to provide a beige solid. The solid was stirred with p-toluenesulfonic acid hydrate (0.38 g) in THF (0.5 mL) at r.t. for 3 days. The mixture was diluted with diethyl ether (5 mL) and then filtered. The solid was washed with diethyl ether and dried in vacuo to afford the title compound; (0.330 g, 75%); as a beige solid; ¹H NMR: 1.97 (2H, m), 2.29 (3H, s), 2.58 (2H, m), 3.14 (2H, m), 3.39 (3H, s), 3.44 (2H, m), 4.60 (1H, m), 7.12 (2H, d), 7.49 (2H, d), 8.39 (1H, s), 8.35-8.77 (2H, m); m/z: MH⁺ 268, 270 (1×Cl).

### Method 39

### 2-Chloro-N-isopropyl-5-nitropyrimidin-4-amine

Using a similar procedure to Method 1 (isopropylamine was used in place of cyclopentylamine) the title compound was prepared; (22.57 g, 98%); as a light brown solid; m/z: (M-H)⁻=215; HPLC Rₜ=2.12 min.

### Method 40

### 2-Chloro-N⁴-isopropylpyrimidine-4,5-diamine

Using a similar procedure to Method 2 (2-chloro-N-isopropyl-5-nitropyrimidin-4-amine (Method 39) was used in place of 2-chloro-N-cyclopentyl-5-nitro-pyrimidin-4-amine) the title compound was prepared; (16.1 g, 89%); as a purple gum. ¹H NMR: (CDCl₃) 1.26 (6H, d), 2.04-2.97 (2H, m), 4.29-4.37 (1H, m), 4.91 (1H, s), 7.59 (1H, s); m/z: MH⁺ 185, 189 (1×Cl).

### Method 41

### 2-Chloro-9-isopropyl-7,9-dihydro-8H-purin-8-one

2-Chloro-N⁴-isopropylpyrimidine-4,5-diamine (Method 40, 16.05 g) was stirred in EtOAc (250 mL) with NaHCO₃ (21.67 g) and water (100 mL) in an ice/salt bath. Phenyl chloroformate (16.18 mL) was added dropwise over 10 mins, then the mixture was warmed to r.t. and stirred overnight. The mixture was then heated at 70° C. for 1.5 h. After cooling, EtOAc (200 mL) was added. The organic layer was separated, washed with 1M HCl (100 mL), dried (MgSO₄) and concentrated. The solid obtained was triturated with DCM (200 mL), filtered and washed with DCM to afford the title compound; (9.13 g, 50%); as a pale brown solid. The filtrate was purified by FCC, eluting with (0-50% EtOAc) in DCM to provide more of the title compound; (1.70 g, 9%); as a solid; ¹H NMR: 1.48 (6H, d), 4.50-4.63 (1H, m), 8.12 (1H, s), 11.55 (1H, s); m/z: MH⁺ 213, 215 (1×Cl).

### Method 42

### 2-Chloro-9-isopropyl-7-methyl-7,9-dihydro-8H-purin-8-one

Using a similar procedure to Method 4 (2-Chloro-9-isopropyl-7,9-dihydro-8H-purin-8-one (Method 41) was used in place of 2-chloro-9-cyclopentyl-7H-purin-8-one) the title compound was prepared; (8.5 g, 87%); as a solid; ¹H NMR: 1.48 (6H, d), 3.36 (3H, s), 4.55-4.68 (1H, m), 8.34 (1H, s); m/z: MH⁺ 227, 229 (1×Cl).

1. A compound of formula (I): 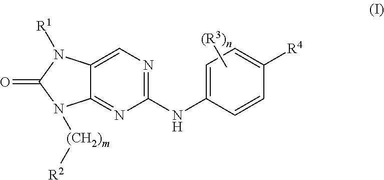
wherein:
R¹ is selected from C₁₋₄alkyl, cyclopropyl, cyclopropylmethyl and cyclobutyl; wherein said cyclopropyl may be optionally substituted by methyl; and wherein R¹ may be optionally substituted by one or more R⁵;
m is 0 or 1;
R² is selected from C₁₋₆alkyl, C₂₋₆alkenyl, C₂₋₆alknyl, C₃₋₆cycloalkyl, cyclopentenyl, cyclohexenyl, oxetanyl, tetrahydropyranyl, tetrahydrofuranyl, oxepanyl, azetidinyl, pyrrolidinyl, piperidinyl and azepanyl; wherein R² may be optionally substituted on carbon by one or more R⁶; and wherein if R² contains a ring —NH— moiety, that nitrogen may be optionally substituted by R⁷;
R³ is independently selected from fluoro, chloro, bromo, cyano, methoxy, ethoxy, trifluoromethoxy, methyl, ethyl, trifluoromethyl, ethenyl, ethynyl, cyclopropyl, methylthio, ethylthio, N-methylamino, N,N-dimethylamino, amino and methylsulfonyloxy;
n is an integer selected from 0 to 3; wherein the values of R³ may be the same or different;
R⁴ is -L-R⁸ or R⁹;
L is selected from ethynylene, ethenylene, cyclopropyl and —X—C₁₋₂alkylene-; wherein X is a direct bond, —O—, —S—, —NH—, —OS(O)₂—, —N(CH₃)— or —N(CH₂R¹⁰)—; and wherein L may be optionally substituted on carbon by one or more fluoro;
R⁵ is cyano or fluoro;
R⁶ is selected from C₁₋₃alkyl, C₁₋₃alkoxy, N—(C₁₋₃alkyl)amino, N,N—(C₁₋₃alkyl)₂amino, hydroxy, amino, fluoro and cyano;
R⁷ is selected from C₁₋₃alkyl, cyclopropyl, C₁₋₃alkanoyl and C₁₋₃alkylsulfonyl;
R⁸ and R¹⁰ are each independently selected from chloro, bromo, iodo, cyano, nitro, mercapto, sulfo, hydroxy, carboxy, amino, carbamoyl, sulfamoyl, C₂₋₆alkyl, C₂₋₆alkenyl, C₂₋₆alkynyl, C₁₋₆alkoxy, C₁₋₆alkylsulfonyloxy, N—(C₁₋₆alkyl)sulfamoyloxy, N,N—(C₁₋₆alkyl)₂sulfamoyloxy, C₁₋₆alkoxycarbonyl, C₁₋₆alkanoyl, C₁₋₆alkanoyloxy, N—(C₁₋₆alkyl)amino, N,N—(C₁₋₆alkyl)₂amino, N—(C₁₋₆alkanoyl)-N—(R¹¹)amino, N—(C₁₋₆alkoxycarbonyl)-N—(R¹²)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—[(C₁₋₆alkyl)sulfonyl]-N—(R¹³)amino, (N,N—(R¹⁴)(R¹⁵)sulfamoyl)-N—(R¹⁶)amino, 3,3-(R¹⁷)(R¹⁸)-1-(R¹⁹)ureido, carbocyclyl-R²⁰—, heterocyclyl-R²¹— and (C₁₋₆alkyl)-S(O)ₐ— wherein a is 0 to 2; wherein R⁸ and R¹⁰ may be optionally substituted on carbon by one or more R²²; and wherein if said heterocyclyl has an —NH— moiety, that nitrogen may be optionally substituted by R²³;
R⁹ is selected from carboxy, carbamoyl, sulfamoyl, C₃₋₆alkyl, C₃₋₆alkenyl, C₃₋₆alkynyl, C₃₋₆alkoxy, C₁₋₆alkylsulfonyl, C₁₋₆alkylsulfinyl, C₃₋₆alkylsulfanyl, C₂₋₆alkylsulfonyloxy, N—(C₁₋₆alkyl)sulfamoyloxy, N,N—(C₁₋₆alkyl)₂sulfamoyloxy, C₁₋₆alkoxycarbonyl, C₁₋₆alkanoyl, C₁₋₆alkanoyloxy, N—(C₂₋₆alkyl)amino, N,N—(C₂₋₆alkyl)₂amino, N—(C₁₋₆alkanoyl)-N—(R²⁴)amino, N—(C₁₋₆alkoxycarbonyl)-N—(R²⁵)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—[(C₁₋₆alkyl)sulfonyl]-N—(R²⁶)amino, (N,N—(R²⁷)(R²⁸)sulfamoyl)-N—(R²⁹)amino, 3,3-(R³⁰)(R³¹)-1-(R³²)ureido, C₄₋₁₂-carbocyclyl-R³³— and heterocyclyl-R³⁴—; wherein R⁹ may be optionally substituted on carbon by one or more R³⁵, and wherein if said heterocyclyl has an —NH— moiety, that nitrogen may be optionally substituted by R³⁶;
R²² and R³⁵ are independently selected from halo, cyano, nitro, mercapto, sulfo, hydroxy, carboxy, amino, carbamoyl, sulfamoyl, C₁₋₆alkyl, C₂₋₆alkenyl, C₂₋₆alkynyl, C₁₋₆alkoxy, C₁₋₆alkylsulfonyloxy, N—(C₁₋₆alkyl)sulfamoyloxy, N,N—(C₁₋₆alkyl)₂sulfamoyloxy, C₁₋₆alkoxycarbonyl, C₁₋₆alkanoyl, C₁₋₆alkanoyloxy, N—(C₁₋₆alkyl)amino, N,N—(C₁₋₆alkyl)₂amino, N—(C₁₋₆alkanoyl)-N—(R³⁷)amino, N—(C₁₋₆alkoxycarbonyl)-N—(R³⁸)amino, N—(C₁₋₆alkyl)carbamoyl, N,N—(C₁₋₆alkyl)₂carbamoyl, N—(C₁₋₆alkyl)sulfamoyl, N,N—(C₁₋₆alkyl)₂sulfamoyl, N—[(C₁₋₆alkyl)sulfonyl]-N—(R³⁹)amino, (N,N—(R⁴⁰)(R⁴¹)sulfamoyl)-N—(R⁴²)amino, 3,3-(R⁴³)(R⁴⁴)-1-(R⁴⁵)ureido, carbocyclyl-R⁴⁶—, heterocyclyl-R⁴⁷—, and (C₁₋₆alkyl)-S(O)ₐ— wherein a is 0 to 2; wherein R²² and R³⁵ may be optionally substituted on carbon by one or more R⁴⁸; and wherein if said heterocyclyl has an —NH— moiety, that nitrogen may be optionally substituted by R⁴⁹;
R²³ and R³⁶ are independently selected from C₁₋₆alkyl, C₂₋₆alkenyl, C₂₋₆alkynyl, C₁₋₆alkoxycarbonyl, C₁₋₆alkanoyl, carbamoyl, N—(C₁₋₆alkyl)carbamoyl, N,N—(C₁₋₆alkyl)₂carbamoyl, sulfamoyl, N—(C₁₋₆alkyl)sulfamoyl, N,N—(C₁₋₆alkyl)₂sulfamoyl, carbocyclyl-R⁵⁰—, heterocyclyl-R⁵¹—, and (C₁₋₆alkyl)-S(O)ₐ— wherein a is 1 or 2; wherein R²³ and R³⁶ may be independently optionally substituted on carbon by one or more R⁵²; and wherein if said heterocyclyl contains an —NH— moiety that nitrogen may be optionally substituted by R⁵³;
R²⁰ and R²¹ are each independently selected from a direct bond, —O—, —N(R⁵⁴)—, —C(O)—, —N(R⁵⁵)C(O)—, —C(O)N(R⁵⁶)—, —SO₂N(R⁵⁷)—, —N(R⁵⁸)—C(O)—N(R⁵⁹)—, —OS(O)₂—, —S(O)₂O—, —N(R⁶⁰)S(O)₂N(R⁶¹)—, —N(R⁶²)SO₂— and —S(O)ₐ— wherein a is 0 to 2;
R³³ and R³⁴ are each independently selected from a direct bond, —O—, —N(R⁶³)—, —C(O)—, —N(R⁶⁴)C(O)—, —C(O)N(R⁶⁵)—, —SO₂N(R⁶⁶)—, —N(R⁶⁷)—C(O)—N(R⁶⁸)—, —OS(O)₂—, —S(O)₂O—, —N(R⁶⁹)S(O)₂N(R⁷⁰)—, —N(R⁷¹)SO₂— and —S(O)ₐ— wherein a is 0 to 2;
R⁴⁶ and R⁴⁷ are each independently selected from a direct bond, —O—, —N(R⁷²)—, —C(O)—, —N(R⁷³)C(O)—, —C(O)N(R⁷⁴)—, —SO₂N(R⁷⁵)—, —N(R⁷⁶)—C(O)—N(R⁷⁷)—, —OS(O)₂—, —S(O)₂O—, —N(R⁷⁸)S(O)₂N(R⁷⁹)—, —N(R⁸⁰)SO₂— and —S(O)ₐ— wherein a is 0 to 2;
R⁵⁰ and R⁵¹ are each independently selected from a direct bond, —C(O)—, —N(R⁸¹)C(O)—, —N(R⁸²)SO₂—, —O—C(O)— and —S(O)ₐ— wherein a is 1 or 2;
R⁴⁸ and R⁵² are each independently selected from fluoro, chloro, cyano, nitro, hydroxy, trifluoromethoxy, trifluoromethyl, amino, carboxy, sulfo, carbamoyl, mercapto, sulfamoyl, methyl, ethyl, ethenyl, methoxy, ethoxy, formyl, acetyl, acetoxy, N-methylamino, N-ethylamino, N,N-dimethylamino, N,N-diethylamino, N-ethyl-N-methylamino, N-formylamino, N-acetylamino, N-methylcarbamoyl, N-ethylcarbamoyl, N,N-dimethylcarbamoyl, N,N-diethylcarbamoyl, N-ethyl-N-methylcarbamoyl, methylsulfanyl, ethylsulfanyl, methylsulfinyl, ethylsulfinyl, methylsulfonyl, methylsulfonyloxy, ethylsulfonyl, ethylsulfonyloxy, methoxycarbonyl, ethoxycarbonyl, N-methylsulfamoyl, N-ethylsulfamoyl, N,N-dimethylsulfamoyl, N,N-diethylsulfamoyl and N-ethyl-N-methylsulfamoyl;
R⁴⁹ and R⁵³ are each independently selected from C₁₋₆alkyl, C₃₋₆cycloalkyl, C₁₋₆alkanoyl, C₁₋₆alkylsulfonyl, C₁₋₆alkoxycarbonyl, carbamoyl, N—(C₁₋₆alkyl)carbamoyl, N,N—(C₁₋₆alkyl)₂carbamoyl, benzyl, benzyloxycarbonyl, benzoyl and phenylsulfonyl;
R¹¹, R¹², R¹³, R¹⁴, R¹⁵, R¹⁶, R¹⁷, R¹⁸, R¹⁹, R⁵⁴, R⁵⁵, R⁵⁶, R⁵⁷, R⁵⁸, R⁵⁹, R⁶⁰, R⁶¹ and R⁶² are each independently hydrogen or a group selected from C₁₋₃alkyl and cyclopropyl wherein said group may be optionally substituted on carbon by one or more R²²;
R²⁴, R²⁵, R²⁶, R²⁷, R²⁸, R²⁹, R³⁰, R³¹, R³², R⁶³, R⁶⁴, R⁶⁵, R⁶⁶, R⁶⁷, R⁶⁸, R⁶⁹, R⁷⁰ and R⁷¹ are each independently hydrogen or a group selected from C₁₋₃alkyl and cyclopropyl wherein said group may be optionally substituted on carbon by one or more R³⁵;
R³⁷, R³⁸, R³⁹, R⁴⁰, R⁴¹, R⁴², R⁴³, R⁴⁴, R⁴⁵, R⁷², R⁷³, R⁷⁴, R⁷⁵, R⁷⁶, R⁷⁷, R⁷⁸, R⁷⁹ and R⁸⁰ are each independently hydrogen or a group selected from C₁₋₃alkyl and cyclopropyl wherein said group may be optionally substituted on carbon by one or more R⁴⁸;
R⁸¹ and R⁸² are each independently hydrogen or a group selected from C₁₋₃alkyl and cyclopropyl wherein said group may be optionally substituted on carbon by one or more R⁵²;
or a pharmaceutically acceptable salt thereof;
wherein the compound of formula (I) is other than:
2-{[4-(4-acetylpiperazin-1-yl)phenyl]amino}-7-methyl-9-pentan-3-yl-7,9-dihydro-8H-purin-8-one or
7-methyl-2-{[4-(4-methylpiperazin-1-yl)phenyl]amino-}-9-pentan-3-yl-7,9-dihydro-8H-purin-8-one;
or a pharmaceutically acceptable salt thereof.

2. The compound of formula (I), or a pharmaceutically acceptable salt thereof, as claimed in claim 1, wherein m is 0.

3. The compound of formula (I), or a pharmaceutically acceptable salt thereof, as claimed in claim 1, wherein:
R¹ is C₁₋₄alkyl, wherein R¹ may be optionally substituted by one or more R⁵; and
R⁵ is cyano.

4. The compound of formula (I), or a pharmaceutically acceptable salt thereof, as claimed in claim 1, wherein:
R² is C₁₋₆alkyl, C₃₋₆cycloalkyl or piperidinyl.

5. The compound of formula (I), or a pharmaceutically acceptable salt thereof, as claimed in claim 1, wherein:
R³ is independently selected from fluoro, chloro, methoxy, ethoxy and methyl.

6. The compound of formula (I), or a pharmaceutically acceptable salt thereof, as claimed in claim 1, wherein:
R⁴ is -L-R⁸ or R⁹;
L is —X—C₁₋₂alkylene—wherein X is a direct bond or —O—;
R⁸ is N,N—(C₁₋₆alkyl)₂amino, carbocyclyl or heterocyclyl and wherein if said heterocyclyl has an —NH— moiety, that nitrogen may be optionally substituted by R²³;
R⁹ is selected from carboxy, sulfamoyl, C₃₋₆alkoxy, C₁₋₆alkylsulfonyl, N—(C₁₋₆alkyl)carbamoyl, N,N—(C₁₋₆alkyl)₂carbamoyl, N—[(C₁₋₆alkyl)sulfonyl]amino, C₄₋₁₂carbocyclyl-R³³— and heterocyclyl-R³⁴—, wherein R⁹ may be optionally substituted on carbon by one or more R³⁵, and wherein if said heterocyclyl has an —NH— moiety, that nitrogen may be optionally substituted by R³⁶;
R³⁵ are independently selected from N,N—(C₁₋₆alkyl)₂amino and heterocyclyl; wherein if said heterocyclyl has an —NH— moiety, that nitrogen may be optionally substituted by R⁴⁹;
R²³ and R³⁶ are independently selected from C₁₋₆alkyl and heterocyclyl wherein R²³ and R³⁶ may be independently optionally substituted on carbon by one or more R⁵²; and wherein if said heterocyclyl contains an —NH— moiety, that nitrogen may be optionally substituted by R⁵³;
R³³ and R³⁴ are each independently selected from a direct bond, —O—, —NH—. —C(O)—, —NH—C(O)— and —SO₂—;
R⁵² is methoxy; and
R⁴⁹ and R⁵³ are each independently C₁₋₆alkyl.

7. The compound of formula (I), or a pharmaceutically acceptable salt thereof, as claimed in claim 1, wherein:
m is 0;
R¹ is C₁₋₄alkyl, wherein R¹ may be optionally substituted by one or more R⁵;
R⁵ is cyano;
R² is C₁₋₆alkyl, C₃₋₆cycloalkyl or piperidinyl;
R³ is independently selected from fluoro, chloro, methoxy, ethoxy and methyl;
n is an integer selected from 0 to 3; wherein the values of R³ may be the same or different;
R⁴ is -L-R⁸ or R⁹;
L is —X—C₁₋₂alkylene—wherein X is a direct bond or —O—;
R⁸ is N,N—(C₁₋₆alkyl)₂amino, carbocyclyl or heterocyclyl and wherein if said heterocyclyl has an —NH— moiety, that nitrogen may be optionally substituted by R²³;
R⁹ is selected from carboxy, sulfamoyl, C₃₋₆alkoxy, C₁₋₆alkylsulfonyl, N—(C₁₋₆alkyl)carbamoyl, N,N—(C₁₋₆alkyl)₂carbamoyl, N—[(C₁₋₆alkyl)sulfonyl]amino, C₄₋₁₂-carbocyclyl-R³³— and heterocyclyl-R³⁴—, wherein R⁹ may be optionally substituted on carbon by one or more R³⁵, and wherein if said heterocyclyl has an —NH— moiety, that nitrogen may be optionally substituted by R³⁶;
R³⁵ are independently selected from N,N—(C₁₋₆alkyl)₂amino and heterocyclyl; wherein if said heterocyclyl has an —NH— moiety, that nitrogen may be optionally substituted by R⁴⁹;
R²³ and R³⁶ are independently selected from C₁₋₆alkyl and heterocyclyl wherein R²³ and R³⁶ may be independently optionally substituted on carbon by one or more R⁵²; and wherein if said heterocyclyl contains an —NH— moiety, that nitrogen may be optionally substituted by R⁵³;
R³³ and R³⁴ are each independently selected from a direct bond, —O—, —NH—. —C(O)—, —NH—C(O)— and —SO₂—;
R⁵² is methoxy; and
R⁴⁹ and R⁵³ are each independently C₁₋₆alkyl.

8. The compound of formula (I), or a pharmaceutically acceptable salt thereof, as claimed in claim 1, wherein:
R¹ is selected from methyl, ethyl and cyanomethyl;
R² is isopropyl, cyclopentyl or piperidin-4-yl;
m is 0;
R³ is independently selected from fluoro, chloro, methoxy, ethoxy and methyl;
n is an integer selected from 0 to 2; wherein the values of R³ may be the same or different;
R⁴ is selected from: N-(1-methylpiperidin-4-yl)carbamoyl, sulfamoyl, mesyl, 4-methylpiperazin-1-yl, 1-methylpiperidin-4-yloxy, morpholin-4-yl, mesylamino, pyrrolidin-1-ylcarbonyl, N-(1-methylpiperidin-4-yl)carbamoyl, methylcarbamoyl, 1,1-dioxo-1,4-thiazinan-4-yl, (4-methylpiperazin-1-yl)sulfonyl, [(9-methyl-9-azabicyclo[3.3.1]non-3-yl)amino]carbonyl, N-(1-ethylpiperidin-4-yl)carbamoyl, 4-methyl-1,4-diazepan-1-yl, 2-hydroxyethyl, 1-methylpiperidin-4-ylamino, 4-(dimethylamino)piperidin1-yl, piperidin-1-yl, benzyl (1-methylpyrrolidin-3-yl)oxy, 2-(dimethylamino)ethoxy, 2-(4-methylpiperazin-1-yl)ethyl, 1-methylpiperidin-4-yl, 4-ethylpiperazin-1-yl, carboxy, (4-methylpiperazin-1-yl)carbonyl, 4-(1-methylpiperidin-4-yl)piperazin-1-ylcarbonyl, 3-(imidazol-1-yl)propylcarbamoyl, N-methyl-N-[(1-isopropylpyrrolidin-3-yl)methyl]-carbamoyl, dimethylcarbamoyl, N-methyl-N-(3-dimethylaminopropyl)carbamoyl, benzoyl, isopropoxy, phenoxy, 3-(dimethylamino)pyrrolidin-1-ylcarbonyl, 4-(pyrrolidin-1-yl)piperidin-1-ylcarbonyl, 4-(2-methoxyethyl)piperazin-1-ylcarbonyl, (4-dimethylaminocyclohexyl)carbamoyl, [1-(2-methoxyethyl)piperidin-4-yl]carbamoyl, pyrrolidin-3-ylcarbamoyl, oxazol-5-yl, N-[1-ethylpyrrolidin-2-yl)methyl]carbamoyl, N-[4-(dimethylamino)butyl]carbamoyl, N-[3-(dimethylamino)propyl]carbamoyl, N-[2-(piperidin-1-yl)ethyl]carbamoyl, N-[2-(4-methylpiperazin-1-yl)ethyl]carbamoyl, N-[4-(pyrrolidin-1-yl)butyl]carbamoyl, N-[2-(dimethylamino)ethyl]carbamoyl and pyrazol-1-yl.

9. The compound of formula (I), or a pharmaceutically acceptable salt thereof, as claimed in claim 1 wherein:
the compound of formula (I) is a compound of formula (IA): 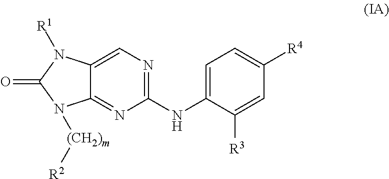
wherein:
R³ is methoxy or ethoxy; and
the values of R¹, R², m, and R⁴ are as defined in claim 1.

10. The compound of formula (I) of formula (IA), or a pharmaceutically acceptable salt thereof, as claimed in claim 9, wherein:
R¹ is selected from methyl, ethyl and cyanomethyl;
R² is isopropyl, cyclopentyl or piperidin-4-yl;
m is 0;
R⁴ is selected from: N-(1-methylpiperidin-4-yl)carbamoyl, sulfamoyl, mesyl, 4-methylpiperazin-1-yl, 1-methylpiperidin-4-yloxy, morpholin-4-yl, mesylamino, pyrrolidin-1-ylcarbonyl, N-(1-methylpiperidin-4-yl)carbamoyl, methylcarbamoyl, 1,1-dioxo-1,4-thiazinan-4-yl, (4-methylpiperazin-1-yl)sulfonyl, [(9-methyl-9-azabicyclo[3.3.1]non-3-yl)amino]carbonyl, N-(1-ethylpiperidin-4-yl)carbamoyl, 4-methyl-1,4-diazepan-1-yl, 2-hydroxyethyl, 1-methylpiperidin-4-ylamino, 4-(dimethylamino)piperidin1-yl, piperidin-1-yl, benzyl (1-methylpyrrolidin-3-yl)oxy, 2-(dimethylamino)ethoxy, 2-(4-methylpiperazin-1-yl)ethyl, 1-methylpiperidin-4-yl, 4-ethylpiperazin-1-yl, carboxy, (4-methylpiperazin-1-yl)carbonyl, 4-(1-methylpiperidin-4-yl)piperazin-1-ylcarbonyl, 3-(imidazol-1-yl)propylcarbamoyl, N-methyl-N-[(1-isopropylpyrrolidin-3-yl)methyl]-carbamoyl, dimethylcarbamoyl, N-methyl-N-(3-dimethylaminopropyl)carbamoyl, benzoyl, isopropoxy, phenoxy, 3-(dimethylamino)pyrrolidin-1-ylcarbonyl, 4-(pyrrolidin-1-yl)piperidin-1-ylcarbonyl, 4-(2-methoxyethyl)piperazin-1-ylcarbonyl, (4-dimethylaminocyclohexyl)carbamoyl, [1-(2-methoxyethyl)piperidin-4-yl]carbamoyl, pyrrolidin-3-ylcarbamoyl, oxazol-5-yl, N-[1-ethylpyrrolidin-2-yl)methyl]carbamoyl, N-[4-(dimethylamino)butyl]carbamoyl, N-[3-(dimethylamino)propyl]carbamoyl, N-[2-(piperidin-1-yl)ethyl]carbamoyl, N-[2-(4-methylpiperazin-1-yl)ethyl]carbamoyl, N-[4-(pyrrolidin-1-yl)butyl]carbamoyl, N-[2-(dimethylamino)ethyl]carbamoyl and pyrazol-1-yl.

11. A pharmaceutical composition comprising a compound of formula (I), or a pharmaceutically acceptable salt thereof, as claimed in claim 1, in association with a pharmaceutically acceptable adjuvant, diluent or carrier.

12-14. (canceled)

15. A method of inhibiting TTK in a mammal comprising contacting a compound of formula (I), or a pharmaceutically acceptable salt thereof, as claimed in claim 1 with the TTK enzyme.

16. A method of treating cancer in a warm-blooded animal comprising administering an effective amount of a compound of formula (I), or a pharmaceutically acceptable salt thereof, as claimed in claim 1 to the warm-blooded animal.
